(19) United States
(12) Patent Application Publication
(10) Pub. No.: US 2026/0001937 A1
(43) Pub. Date: Jan. 1, 2026

(54) NOVEL RECOMBINANT FIBRINOGEN VARIANTS FOR FIBRIN SEALANTS FOR SURGICAL WOUND CARE

(71) Applicant: Julius-Maximilians-Universität Würzburg, Würzburg (DE)

(72) Inventors: Lorenz MEINEL, Wuerzburg (DE); Marc DRIESSEN, Krefeld (DE); Matthias BEUDERT, Wuerzburg (DE); Tessa LUEHMANN, Wuerzburg (DE); Rafael WORSCHECH, Wuerzburg (DE)

(73) Assignee: Julius-Maximilians-Universität Würzburg, Würzburg (DE)

(21) Appl. No.: 18/866,603

(22) PCT Filed: May 17, 2023
(86) PCT No.: PCT/EP2023/063273

§ 371 Date: Nov. 18, 2024

(30) Foreign Application Priority Data
May 17, 2022 (EP) 22173918.8

(51) Int. Cl. C07K 14/75; A61K 38/00; C12P 21/00

(52) U.S. Cl. CPC C07K 14/75; C12P 21/005; A61K 38/00

(57) ABSTRACT

The invention relates a novel isolated recombinant fibrinogen and to a eucaryotic cell producing said novel recombinant fibrinogen, as well as to a fibrin sealant and a fibrin sealant kit comprising the novel isolated recombinant fibrinogen. Furthermore, the present invention relates to a new method for the production of recombinant fibrinogen using eucaryotic cells, and to a new method for identifying novel recombinant fibrinogen variants with reduced sensitivity to proteolytic cleavage by plasmin without losing their suitability as fibrinogen component in a fibrin sealant.

### RELATED APPLICATIONS

This application is a National Phase of PCT Patent Application No. PCT/EP2023/063273 having International filing date of May 17, 2023, which claims the benefit of priority of European Patent Application No. 22173918.8 filed on May 17, 2022. The contents of the above applications are all incorporated by reference as if fully set forth herein in their entirety.

### SEQUENCE LISTING STATEMENT

The XML file, entitled 101993SequenceListing.xml, created on Nov. 18, 2024, comprising 84,186 bytes, submitted concurrently with the filing of this application is incorporated herein by reference. The sequence listing submitted herewith is identical to the sequence listing forming part of the international application.

### FIELD AND BACKGROUND OF THE INVENTION

An estimated 313 million surgeries are performed globally every year.¹ As the population grows to approximately 9.73 billion people in the future, the number of surgical procedures required will also increase.² A major drawback of these medical interventions is the risk of severe bleeding, which can lead to increased morbidity and death.³ This risk is particularly high in soft tissue surgical procedures, of which approximately 7% result in severe bleeding.^{4, 5} Although there have been immense advancements in surgical techniques over the past few decades, uncontrolled bleeding is still a major problem, especially in more extensive procedures such as hepatectomy.⁶⁻¹⁰ for example, 2-6% of all patients undergoing cardiac surgery present with intraoperative or postoperative bleeding and have an increased mortality rate as a result. Intraoperative or postoperative bleeding is, however, not only a burden for patients, but also for the entire healthcare system, as it is associated with significant costs due to an increased likelihood of second surgeries and prolonged hospitalization.¹¹⁻¹³

A variety of different methods and applications are used to control bleeding and improve hemostasis during medical procedures. Standard methods include the use of sutures, clips, and staples with estimated annual sales of up to $15 billion by 2024.⁴ Even though these methods have been established for a long time and in many cases show the desired effects, there are still problems and shortcomings. In more challenging procedures, up to 30% of patients experience fluid leakage after surgery.¹⁴ In addition, sutures used for surgical wound management are often susceptible to bacterial growth and can lead to microbial infections at the surgical site.¹⁵⁻¹⁶

Over the past 20 years, a variety of tissue sealants, hemostats, and tissue adhesives have been developed as alternative technologies to address these challenges. To date, these approaches have proven to be valuable additions to the current toolbox. The emerging importance of adhesives and sealants is reflected by a market volume with a current value of $10.3 billion in 2020 and a projected compound annual growth rate of 5.6%.⁴ These hemostatic materials are primarily based on the approach of mimicking and supporting the natural mechanism of hemostasis in the body.

Over the years, a variety of these new technologies have been developed, particularly for use in superficial wounds, such as pressure dressings, gels, and foams.¹⁷⁻²¹ However, this trend is not currently emerging for soft tissue and internal wound procedures. In these applications, new materials must withstand extreme challenges such as moist environments and dynamic forces. For these types of surgeries, blood transfusion remains the standard method.²² However, the use of blood transfusion is accompanied by several problems, such as limited donor availability, potential immunogenicity, or risk of infection.²³⁻²⁵ Although more and more new approaches are being developed, the transition from laboratory to patient use is a major problem.

The first such material to be approved by the US food and drug administration (FDA) as a hemostat, in 1998, was a so-called fibrin sealant.²⁶ This material works by mimicking and supporting the natural mechanism of hemostasis. Fibrinogen and thrombin form the basis and the main components. These two proteins play the central role in the final step of the blood clotting cascade in the body, leading to the formation of the fibrin clot and thus wound closure.

Fibrinogen is a longitudinal hexameric protein whose shape is reminiscent of a dumbbell. The soluble protein is formed using three subunits, the α-, μ-, and γ-chain. In the native form, the protein forms a hexamer of 2 units of each of the described chains with a molecular weight of approximately 340 kDa in total (Aα-(66.5 kDa), Bβ-(52 kDa), and γ-chain (46.5 kDa)).^{30, 36-37} In nature, fibrinogen exists in heterogenous population of different forms. Both the alpha- and gamma-chain (α- and γ-chain) have different isoforms that are formed due to alternative splicing. The α-chain comprises two different forms, the isoform E (αE), also known as isoform 1 (molecular weight: ˜93 kDa), which herein is referred to as “isoform alpha-E” or “isoform αE”, and the isoform 2 which is in general referred to as α-chain, which herein is referred to as “isoform alpha” or “isoform α”. Fibrinogen comprising isoform αE accounts for around 1-2% of the fibrinogen population in the human body. There are also indications that fibrinogen containing isoform αE might be less susceptible to proteolytic degradation making it an interesting candidate in order to increase the half-life of fibrin clots towards plasmin.³⁸ The term “Aα” or “A alpha” refers to the secreted form of alpha-chain isoform alpha, which lacks the N-terminal amino acids of the signal peptide present in the full-length isoform alpha, but still includes the “Fibrinopeptide A”, which consists of amino acids 20-35 of the full-length amino acid sequence of isoform alpha. Similarly, the term “AαE” or “A alpha-E” refers to the secreted form of alpha-chain isoform alpha-E, which lacks the N-terminal amino acids of the signal peptide present in the full-length amino acid sequence of isoform alpha-E, but still includes the “Fibrinopeptide A” or (FpA), which consists of amino acids 20-35 of the full-length amino acid sequence of isoform alpha-E.

The γ-chain also exists in nature in two different isoforms. The first isoform is referred to as gamma or gamma A (γ or γA; also simply referred to as gamma-chain, or gamma A chain). The second isoform is referred to as gamma′ or gamma B (γ′ or gamma; also simply referred to as gamma′-chain, or gamma B chain). Around 10-15% of fibrinogen in the body consists of a heterodimer (γ/γ′) and only about 0.5% of a homodimer γ′/γ′. However, the presence of isoform γ′ can have an impact on the structure and function of fibrinogen and the fibrin clot.³⁹ During hemostasis the N-termini of both α and β-chain are processed by the enzyme thrombin which leads to the formation of the fibrin network.^{40, 41} Exposure of the protein to thrombin results in the cleavage of the so-called fibrinopeptide A (or FpA) sequence in the N-terminus of the α-chain and of the so-called fibrinopeptide B (FpB) in the N-terminus of the β-chain, initiating the formation of the protein complex that leads to wound closure.³¹ Due to its natural role and mechanics, fibrin has been studied comprehensively during the last decades and has been used as biomaterial for different fields of medicine.^{42, 43} The use in fibrin sealants to facilitate wound closure in surgeries with acute bleeding is one of the most important applications of fibrinogen.^{18, 44-45} In the current generation of fibrin sealants (or fibrin glues), fibrinogen and thrombin are filled separately into a prefilled dual syringe system and applied directly to the bleeding site, with the two components only mixed directly at the site of action.²⁷ Upon contact with the wound, fibrinogen and thrombin interact to form fibrin, which results in a hemostatic effect.²⁸ The success of fibrin adhesives is based on many properties, such as biocompatibility, natural occurrence of the proteins in the body, resorption, and the fact that they do not cause inflammation or necrosis and the possible addition of biological active compounds (e.g. growth factors) through intrinsic protein binding sites.^{18, 44-47}

Nevertheless, these materials also have some disadvantages. Fibrinogen is usually obtained from large pools of human plasma. This creates the possibility of transmission of viral diseases. For example, there are studies from Japan showing that parovirus 19 transmission can occur in up to 20% of treated patients.²⁹ One of the major challenges with fibrin sealants, especially during excessive bleeding, however, is the rapid resorption of fibrin in vivo, due to enzymatic degradation by the serine protease plasmin (i.e. fibrinolysis). Plasmin is the most prominent protease during fibrinolysis. Even though fibrinolysis is an essential process in the body to prevent the formation of intravascular clots or the degradation of the clot during tissue regeneration, the proteases transported by the blood lead to reduced stability of the wound sealant.

Thus, degradation of the fibrin clot is mediated by the endogenous serine protease plasmin. Fibrinogen has plasmin cleavage sites on all 3 chains for this purpose. ^{32, 33} Active plasmin is a serine protease that is derived from its zymogen plasminogen.⁴⁸ After activation, plasmin degrades the fibrin clot rapidly. Fibrinogen used in fibrin sealants is often derived from pooled human plasma and therefore already contains small amounts of plasminogen. Upon contact of the sealant with the injury site, this plasminogen is activated by proteases released from the cells and might amplify the sealant resorption in combination with endogenous plasmin.⁴⁹

Over the years, different approaches have been developed to tackle this problem. Currently, the most successful approach has been the addition of protease inhibitors to the sealant, which locally limit plasmin activity. The most common plasmin inhibitor used for these applications is aprotinin (bovine pancreatic trypsin inhibitor). Aprotinin is peptide serine-protease inhibitor with a molecular weight of 6.5 kDa, that has been shown to successfully inhibit different proteases including plasmin. It is also the only inhibitor that is currently used in fibrin sealants formulations already approved by the FDA (e. g. in TISSEEL®, Baxter).

However, the use of aprotinin also has major shortcomings, such as the risk of allergic as well as anaphylactic shock.²⁷ In addition to that, aprotinin diffuses quickly from the injury site.⁴⁹ Thus, plasmin inhibition only has relatively short time frame.

In light of the above shortcomings of available fibrin sealants, the objective of the invention described below is to provide a fibrin sealant with extended half-life in the presence of the protease plasmin, without requiring inclusion of plasmin inhibitors. In particular, there is a need for a fibrin sealant which combines:
A) the physiological advantages of fibrin sealant in hemostasis;
B) prevention of potential viral transmission by application of the product; and
C) prolongation of the half-life of fibrin without potential allergic reaction through addition of the plasmin inhibitor aprotinin.

In addition, the production steps and costs should be minimized by reducing the components of the system.

Wypasek et al., Thrombosis Research, Vol. 182, October 2019, p. 133-140 discloses a screening study on mutants in Polish patients with bleeding disorder, describing the genetic and clinical characterization of congenital fibrinogen mutations in individual chains in patients using concentration determination, polymerase chain reaction (PCR) and Sanger sequencing.

Wypasek et al., analyzes patients with bleeding disorders and linking these to different mutations, thereby disclosing which point mutations in patients with bleeding disorder correlate with the disease activity. Wypasek et al., does not relate to recombinant fibrinogen. Fibrinogen from plasma derivatives as disclosed in Wypasek et al. is not equivalent to recombinant fibrinogen, as the former may contain protein impurities (plasminogen) and/or viral contaminants. Furthermore, fibrinogen naturally occurs in heterogeneous populations of distinct isoforms, resulting in altered properties. Wypasek et al. does not relate to identifying sites which prolong the stability of fibrin clots/glues, and does not provide information on how the wild type (no mutation) must be changed, such that longer lasting fibrin glues result. Furthermore, Wypasek et al. is not instructive on how a fibrinogen can be recombinantly engineered to show, in contrast to the wild type, sustained stability. Instead, Wypasek et al. is about bleeding disorders resulting in the altered quantity and/or quality of circulating fibrinogens, while fibrinolysis of fibrinogen variants is not investigated.

### SUMMARY OF THE INVENTION

The present inventors investigated ways to enable production of a modified wound adhesive with improved properties, including an increased half-life in the presence of the protease plasmin, thereby ensuring less resorption of the adhesive and consequently a longer-lasting hemostatic effect. In doing so, the present inventors, by using a novel approach based on molecular engineering to limit plasmin-induced cleavage of fibrinogen, found novel recombinant fibrinogen variants, which are less sensitive to plasmin-dependent degradation and thus more stable, or stable for longer periods, in the presence of plasmin. As such, the novel recombinant fibrinogen variants can advantageously be used in fibrin sealants without requiring the addition of plasmin protease inhibitor like aprotinin. Thus, the novel recombinant fibrinogen variants overcome the above-described limitations of fibrin sealants of the prior art.

Furthermore, the present inventors found a new method for the production of isolated recombinant fibrinogen variants, using eucaryotic cells, preferably using mammalian cell culture cells. This method advantageously ensures the recombinant production of fully intact fibrinogen, and in particular fibrinogen alpha-chain, by preventing degradation of rFbg during cell culture-based expression, thereby increasing the yield of recombinant fibrinogen variants that can be produced and isolated using a cell culture-based production system.

Finally, the present inventors developed a novel time resolved screening method allowing for the elucidation of plasmin cleavage sites for a potential amino acid exchange to find new molecularly engineered fibrinogen variants. Thus, this method can be advantageously used to generate a new generation of fibrin sealants with an improved stability towards plasmin degradation. By varying both the number of mutations at plasmin cleavage sites as well as their location in the quaternary structure of the protein, fibrin gel resorption can be fine-tuned for specific application.

The present invention therefore relates to the following embodiments.

[1] An isolated recombinant fibrinogen comprising two fibrinogen alpha-chains, two fibrinogen beta-chains and two fibrinogen gamma-chains, having amino acid sequences derived from human fibrinogen; characterized in that
(a) in the fibrinogen alpha-chains, with reference to the respective positions in SEQ ID NO: 2, one or more lysine (K) and/or arginine (R) residues selected from the group consisting R38, R42, K97, K100, R114, R123, R129, R135, K210, R216, R218, K225, K227, K238, K243, K249, R258, R271, R287, R308, R353, R367, R425, R426, K432, K437, K440, R443, K446, K448, R458, R459, K463, K467, K476, K480, R510, R512, K527, R547, K558, R573, K575, K581, R591, K599, K602, K620, R621, K625, R687 and R847 are substituted or deleted, preferably substituted; and/or
(b) in the fibrinogen beta-chains, with reference to the respective positions in SEQ ID NO: 3, one or more lysine (K) and/or arginine (R) residues selected from the group consisting of R44, K51, K52, R53, R60, R72, K77, K83, R87, K152, R158, K160, K163, K178, R285, K328, R334, K348, K353, K367, K374, K458 and K471 are substituted or deleted preferably substituted; and/or
(c) in the fibrinogen gamma-chains, with reference to the respective positions in SEQ ID NO: 4, one or more lysine (K) and/or arginine (R) residues selected from the group consisting of R40, K79, K84, K88, K111, K114, K185, K199, K238, K292, R301, K382 and K399 are substituted or deleted preferably substituted.

[2] The isolated recombinant fibrinogen of item 1, wherein the one or more substituted or deleted K and R residues in the fibrinogen alpha-chains comprise, or consist of, one or more of R135, K210, K243, R287, R308, R425, R426, K432, K437, K440, K446, K448, R458, R459, K463, K467, K480, R512, R547, K558, R573, K575, R591, K599, K620, R621, K625 and combinations thereof; and/or wherein the one or more substituted or deleted K and R residues in the fibrinogen beta-chains comprise, or consist of, one or more of R44, K51, R53, K77, R158, K160, K178, R334, K348, K353, K367, K374, K458, K471, and combinations thereof; and/or wherein the one or more substituted or deleted K and R residues in the fibrinogen gamma-chains comprise, or consist of, K292, R301, or both.

[3] The isolated recombinant fibrinogen of item 1 or 2, wherein the one or more substituted or deleted K and R residues in the fibrinogen alpha-chains comprise, or consist of, one or more of R38, R42, K97, K100, R114, R123, R129, R135, R216, K225, K238, K249, R258, R271, R443, R510, K527 and K602; and/or wherein the one or more substituted or deleted K and R residues in the fibrinogen beta-chains comprise, or consist of, one or more of K52, R72, K83, K152 and K163; and/or wherein the one or more substituted or deleted K and R residues in the fibrinogen gamma-chains comprise, or consist of, one or more of K79, K84, K88, K111, K114, K185, K382 and K399, preferably K88, K111, K382 and K399.

[4] The isolated recombinant fibrinogen of any of the preceding items, wherein the one or more substituted or deleted K and R residues in the fibrinogen alpha-chains comprise, or consist of K97, K100, R114, R123, R129, R135, K210, R216, K225, K238, K243, K249, R258, R271, R287, R308, R425, R426, K432, K437, K440, R443, K446, R458, R459, K463, K467, K476, K480, R510, R512, K527, R547, K558, R573, K575, R591, K599, K602, K620, K621, K625 and combinations thereof, preferably K97, K100, R123, K225, K238, K243, R510, R512, K527, K599, K602, K620, R621, K625 and combinations thereof, more preferably K100, R123, K225, K238, R510, K527, K602, K620 and combinations thereof; and/or wherein the one or more substituted or deleted K and R residues in the fibrinogen beta-chains comprise, or consist of, R44, K51, K52, R53, R72, K77, K83, K152, R158, K160, K163, K178, R334, K348, K353, K367, K374, K458, K471 and combinations thereof, preferably K51, K52, R53, R72, K77, K152, R158, K160, K163, and combinations thereof, more preferably K52, R72, K152, K160 and combinations thereof; and/or wherein the one or more substituted or deleted K and R residues in the fibrinogen gamma-chains comprise, or consist of, one or more of K79, K84, K88, K111, K114, K382 and K399 and combinations thereof, preferably K79, K84, K88, K111, K382 and K399 and combinations, more preferably K79, K88, K111, K382, K399 and combinations thereof, or K88, K111, K382, K399 and combinations thereof.

[5] The isolated recombinant fibrinogen of any of the preceding items, wherein the one or more substituted or deleted K and R residues in the fibrinogen alpha-chains comprise, or consist of K100, R114, R123, R129, R135, K210, R216, K225, K238, K243, K249, R258, R271, R287, R308, R425, R426, K432, K437, K440, R443, K446, R458, R459, K463, K467, K476, K480, R510, R512, K527, R547, K558, R573, K575, R591, K599, K602, K620, K621 and combinations thereof.

[6] The isolated recombinant fibrinogen of any of the preceding items, wherein the one or more substituted or deleted K and R residues in the fibrinogen alpha-chains comprise, or consist of K100, R114, R123, K225, K238, K249, R258, R271, R287, R308, R425, R426, K432, R443, R458, K467, R510, K527, R547, K558, K575, K599, K602, K621 and combinations thereof; and/or wherein the one or more substituted or deleted K and R residues in the fibrinogen beta-chains comprise, or consist of R44, K51, K52, R72, K83, K152, R158, K160, K163 and combinations thereof; and/or wherein the one or more substituted or deleted K and R residues in the fibrinogen gamma-chains comprise, or consist of, one or more of K79, K88, K111, K382, K399 and combinations thereof.

[7] The isolated recombinant fibrinogen of any of the preceding items, wherein in one or preferably both fibrinogen alpha-chains and/or fibrinogen beta-chains and/or fibrinogen gamma-chains at least two, preferably at least three, more preferably at least four, even more preferably at least five, or at least six, or at least seven, or at least eight, or at least nine, etc., or all, of said K and R residues are deleted or substituted, preferably substituted.

[8] The isolated recombinant fibrinogen of item 1, wherein all K and R residues indicated in item 2, and/or in item 3, and/or in item 4, and/or in item 5, and/or in item 6, are deleted or substituted, preferably substituted.

[9] The isolated recombinant fibrinogen of any of the preceding items, wherein the fibrinogen alpha-, beta-, and gamma-chains, apart from the K and R residues specified in any of items 1 to 8, comprise no further mutations in K and R residues.

[10] The isolated recombinant fibrinogen of any of the preceding items, wherein the one or more K or R residues are substituted with amino acids or isotopes of these amino acids selected from the group consisting of alanine (A), histidine (H), N6-[(2-azidoethoxy)carbonyl]-L-lysine, propargyl-L-lysine, azidohomoalanine, trans-cyclooct-2-en-L-lysine, exo BCN-L-lysine, trans-cyclooct-4-en-L-lysine, pyrrolysine and pyrrolysine analogs (e. g. N-cyclopentyloxycarbonyl-L-lysine), lysine-nitrobenzyl-oxycarbonyl-N^{ε}-L-lysine, O-methyl-L-tyrosin and analogs, methionine (M), isoleucine (1), leucine (L), phenylalanine (F), tryptophane (W), preferably selected from alanine (A), histidine (H), N6-[(2-azidoethoxy)carbonyl]-L-lysine, propargyl-L-lysine, azidohomoalanine, methionine (M), isoleucine (1), leucine (L); more preferably selected from alanine (A) or histidine (H).

[11] The isolated recombinant fibrinogen of any of the preceding items, wherein those of the one or more K or R residues, which are located in an α-helical region, are substituted with amino acids or isotopes of these amino acids selected from the group consisting of alanine (A), histidine (H), N6-[(2-azidoethoxy)carbonyl]-L-lysine, propargyl-L-lysine, azidohomoalanine, methionine (M), isoleucine (1), leucine (L); preferably alanine (A) or histidine (H).

[12] The isolated recombinant fibrinogen of any of the preceding items, wherein the fibrinogen alpha-, beta-, and gamma-chains comprise only substitutions, and no deletions of K or R residues.

[13] The isolated recombinant fibrinogen of any of the preceding items, wherein, in addition to the one or more substituted or deleted K and R residues in the fibrinogen alpha-chain(s), and/or the one or more substituted or deleted K and R residues in the fibrinogen beta-chain(s), and/or the one or more substituted or deleted K and R residues in the fibrinogen gamma-chain(s), all K residues, and optionally all R residues, inside 2, preferably 5, more preferably 10, or 15, or 20 amino acids directly up- and/or downstream of (some or all) said one or more substituted or deleted K and R residues, are also substituted or deleted, preferably substituted.

[14] The isolated recombinant fibrinogen of any of the preceding items, wherein, in addition to the one or more substituted or deleted K and R residues in the fibrinogen alpha-chain(s), and/or the one or more substituted or deleted K and R residues in the fibrinogen beta-chain(s), and/or the one or more substituted or deleted K and R residues in the fibrinogen gamma-chain(s), one or more, for example 1, 2, 3 or 4 amino acids, inside 3, preferably 2, amino acids directly up- and/or downstream of (some or all of) said one or more substituted or deleted K and R residues, are also substituted or deleted, preferably substituted.

[15] The isolated recombinant fibrinogen of any of the preceding items, wherein, in addition to the one or more substituted or deleted K and R residues in the fibrinogen alpha-chain(s), and/or the one or more substituted or deleted K and R residues in the fibrinogen beta-chain(s), and/or the one or more substituted or deleted K and R residues in the fibrinogen gamma-chain(s), one or both amino acids directly adjacent to (some or all) said one or more substituted or deleted K and R residues, are also substituted or deleted, preferably substituted.

[16] The isolated recombinant fibrinogen of any of the preceding items, wherein none of the amino acids, apart from the one or more substituted or deleted K and R residues in the fibrinogen alpha-chain(s), and/or the one or more substituted or deleted K and R residues in the fibrinogen beta-chain(s), and/or the one or more substituted or deleted K and R residues in the fibrinogen gamma-chain(s), one or both amino acids directly adjacent to (some or all) said one or more substituted or deleted K and R residues, are also substituted or deleted.

[17] The isolated recombinant fibrinogen of any of the preceding items, wherein of the
i. fibrinogen alpha-chains, at least one, preferably each, comprise, or consist of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, identity with amino acids 20 to 629 of SEQ ID NO: 1; and/or
ii. fibrinogen beta-chains, at least one, preferably each, comprise, or consist of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, identity with amino acids 31 to 491 of SEQ ID NO: 3; and/or
iii. fibrinogen gamma-chains, at least one, preferably each, comprise, or consist of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, identity with amino acids 27 to 433 of SEQ ID NO: 4.

[18] The isolated recombinant fibrinogen of any of the preceding items, wherein
i. each fibrinogen alpha-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, identity with any of SEQ ID NOs: 6, 7, 8, 41, 42, 43, 45, 46, 47, 48, 49, and 50, preferably with SEQ ID NO: 6, or with SEQ ID NO: 7, or with SEQ ID NO: 8, or with SEQ ID NO: 41, or with SEQ ID NO: 42, or with SEQ ID NO: 43, more preferably with SEQ ID NO: 6, SEQ ID NO: 7, or SEQ ID NO: 8; and/or
ii. each fibrinogen beta-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, identity with SEQ ID NO: 9, or with SEQ ID NO: 44, preferably with SEQ ID NO: 9; and/or
iii. each fibrinogen gamma-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, identity with SEQ ID NO: 10, or with SEQ ID NO: 11.

[19] The isolated recombinant fibrinogen of any of the preceding items, wherein
i. each fibrinogen alpha-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, or 100%, identity with SEQ ID NO: 6 or SEQ ID NO: 7, preferably with SEQ ID NO: 7; and
ii. each fibrinogen beta-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, or 100%, identity with SEQ ID NO: 9; and
iii. each fibrinogen gamma-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, or 100%, identity with SEQ ID NO: 10.

[20] The isolated recombinant fibrinogen of any of the preceding items, wherein each fibrinogen alpha-chain comprises, or consists of, an amino acid sequence having, apart from the one or more substituted or deleted K and R residues, the sequence of any of SEQ ID NOs: 6, 7, 8, 41, 42, 43, 45, 46, 47, 48, 49, and 50, preferably of SEQ ID NO: 6, of SEQ ID NO: 7, or of SEQ ID NO: 8, or of SEQ ID NO: 41, or of SEQ ID NO: 42, or of SEQ ID NO: 43, more preferably of SEQ ID NO: 6, SEQ ID NO: 7, or SEQ ID NO: 8; and/or each fibrinogen beta-chain comprises, or consists of, an amino acid sequence having, apart from the one or more substituted or deleted K and R residues, the sequence of SEQ ID NO: 9, or of SEQ ID NO: 44, preferably of SEQ ID NO: 9; and/or each fibrinogen gamma-chain comprises, or consists of, an amino acid sequence having, apart from the one or more substituted or deleted K and R residues, the sequence of SEQ ID NO: 10, or of SEQ ID NO: 11.

[21] The isolated recombinant fibrinogen of any of the preceding items, wherein each fibrinogen alpha-chain further comprises one or more (for example 1, 2, 3 or 4) substitutions or deletions inside 4, or 3, or 2, amino acids directly up- and/or downstream of at least one (e.g. 1, or 2, or 3, etc.) metalloproteinase (MMP) cleavage site (e.g. the at least one MMP cleavage site, with reference to the respective positions in SEQ ID NO: 2, is at K432).

[22] The isolated recombinant fibrinogen of item 21, wherein the one or more substitutions or deletions inside 4, or 3, or 2, amino acids directly up- and/or downstream of at least one metalloproteinase (MMP) cleavage site in the fibrinogen alpha-chains, with reference to the respective positions in SEQ ID NO: 2, comprise, or consist of, substitution of L433 and/or T435, preferably L433H and/or T435R.

[23] The isolated recombinant fibrinogen of any of the preceding items, which is a, preferably soluble, hexamer, comprising two fibrinogen alpha-chains, two beta-chains and two gamma-chains.

[24] The isolated recombinant fibrinogen of any of the preceding items, which is functional.

[25] The isolated recombinant fibrinogen of any of the preceding items, wherein functional means that the recombinant fibrinogen is biologically active.

[26] The isolated recombinant fibrinogen of any of the preceding items, wherein the recombinant fibrinogen is intact.

[27] A fibrin sealant comprising isolated recombinant fibrinogen according to any of items 1 to 26, and optionally thrombin.

[28] A fibrin sealant comprising functional recombinant fibrinogen, wherein each recombinant fibrinogen preferably comprises two fibrinogen alpha-chains, two fibrinogen beta-chains and two fibrinogen gamma-chains, with amino acid sequences derived from human fibrinogen; the recombinant fibrinogen comprising
i. fibrinogen alpha-chains, preferably each comprising, or consisting of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, identity with any of SEQ ID NOs: 6, 7, 8, 41, 42, 43, 45, 46, 47, 48, 49, and 50, preferably with SEQ ID NO: 6, or with SEQ ID NO: 7, or with SEQ ID NO: 8, or with SEQ ID NO: 41, or with SEQ ID NO: 42, or with SEQ ID NO: 43, more preferably with SEQ ID NO: 6, SEQ ID NO: 7, or SEQ ID NO: 8; and/or
ii. fibrinogen beta-chains, preferably each comprising, or consisting of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, identity with SEQ ID NO: 9, or with SEQ ID NO: 44, preferably with SEQ ID NO: 9; and/or
iii. fibrinogen gamma-chains, preferably each comprising, or consisting of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, identity with SEQ ID NO: 10, or with SEQ ID NO: 11;
   and characterized in that
(a) in one, preferably both, of the fibrinogen alpha-chains, with reference to the respective positions in SEQ ID NO: 2, one or more lysine (K) and/or arginine (R) residues selected from the group consisting R38, R42, K97, K100, R114, R123, R129, R135, K210, R216, R218, K225, K227, K238, K243, K249, R258, R271, R287, R308, R353, R367, R425, R426, K432, K437, K440, R443, K446, K448, R458, R459, K463, K467, K476, K480, R510, R512, K527, R547, K558, R573, K575, K581, R591, K599, K602, K620, R621, K625, R687 and R847 are substituted or deleted; and/or
(b) in one, preferably both, of the fibrinogen beta-chains, with reference to the respective positions in SEQ ID NO: 3, one or more lysine (K) and/or arginine (R) residues selected from the group consisting of R44, K51, K52, R53, R60, R72, K77, K83, R87, K152, R158, K160, K163, K178, R285, K328, R334, K348, K353, K367, K374, K458 and K471 are substituted or deleted; and/or
(c) in one, preferably both, of the fibrinogen gamma-chains, with reference to the respective positions in SEQ ID NO: 4, one or more lysine (K) and/or arginine (R) residues selected from the group consisting of R40, K79, K84, K88, K111, K114, K185, K199, K238, K292, R301, K382 and K399 are substituted or deleted.

[29] The fibrin sealant according to item 27 or 28, further comprising, preferably human or bovine, more preferably human, thrombin; and optionally one or more additives selected from the group consisting of protransglutaminase, preferably factor XIII (FXIII), calcium chloride, polyphosphate (PolyP), Zn2+, fibronectin, and hydroxyapatite.

[30] The fibrin sealant according to any of items 27 to 29, wherein each recombinant fibrinogen protein is a, preferably soluble, hexamer comprising two fibrinogen alpha-chains, two beta-chains and two gamma-chains.

[31] The fibrin sealant according to any of items 27 to 30, wherein the isolated recombinant fibrinogen is functional.

[32] The fibrin sealant according to any of items 27 to 31, wherein functional means that the recombinant fibrinogen is biologically active.

[33] The fibrin sealant according to any of items 27 to 32, wherein the recombinant fibrinogen is intact.

[34] The fibrin sealant according to any of items 27 to 33 not comprising aprotinin.

[35] The fibrin sealant according to any of items 27 to 34 not comprising plasmin inhibitors.

[36] The fibrin sealant according to any of items 27 to 35 not comprising protease inhibitors.

[37] The fibrin sealant according to any of items 27 to 36 for use in sealing a defect site or an incised surface of the organs and tissues, or for sealing junction between incised tissues or between incised tissues and prosthetic materials.

[38] A fibrin sealant kit comprising:
i) a container comprising isolated recombinant fibrinogen according to any of items 1 to 26; and
ii) a container comprising, preferably human- or bovine-derived, more preferably human, thrombin.

[39] A fibrin sealant kit comprising:
i) a container comprising functional recombinant fibrinogen, wherein each recombinant fibrinogen protein comprises two fibrinogen alpha-chains, two fibrinogen beta-chains and two fibrinogen gamma-chains; and
ii) a container comprising, preferably human- or bovine-derived, more preferably human, thrombin;
   characterized in that
(a) in one, preferably both, of the fibrinogen alpha-chains, with reference to the respective positions in SEQ ID NO: 2, one or more lysine (K) and/or arginine (R) residues selected from the group consisting R38, R42, K97, K100, R114, R123, R129, R135, K210, R216, R218, K225, K227, K238, K243, K249, R258, R271, R287, R308, R353, R367, R425, R426, K432, K437, K440, R443, K446, K448, R458, R459, K463, K467, K476, K480, R510, R512, K527, R547, K558, R573, K575, K581, R591, K599, K602, K620, R621, K625, R687 and R847 are substituted or deleted; and/or
(b) in one, preferably both, of the fibrinogen beta-chains, with reference to the respective positions in SEQ ID NO: 3, one or more lysine (K) and/or arginine (R) residues selected from the group consisting of R44, K51, K52, R53, R60, R72, K77, K83, R87, K152, R158, K160, K163, K178, R285, K328, R334, K348, K353, K367, K374, K458 and K471 are substituted or deleted; and/or
(c) in one, preferably both, of the fibrinogen gamma-chains, with reference to the respective positions in SEQ ID NO: 4, one or more lysine (K) and/or arginine (R) residues selected from the group consisting of R40, K79, K84, K88, K111, K114, K185, K199, K238, K292, R301, K382 and K399 are substituted or deleted.

[40] The fibrin sealant kit according to item 38 or 39, further comprising at least one additive selected from the group consisting of protransglutaminase, preferably factor XIII (FXIII), one or more calcium salts, preferably calcium chloride, polyphosphate (PolyP), Zn2+, fibronectin, hydroxyapatite, growth factors, preferably VEGF, one or more mono- and/or polysaccharides and cells, preferably stem cells.

[41] The fibrin sealant kit according to item 40, wherein said Factor XIII is included in a container comprising fibrinogen.

[42] The fibrin sealant kit according to any of items 38 to 41 not comprising aprotinin.

[43] The fibrin sealant kit according to any of items 38 to 42, not comprising plasmin inhibitors.

[44] The fibrin sealant kit according to any of items 38 to 43, not comprising protease inhibitors.

[45] The fibrin sealant of any of items 27 to 37, or the fibrin sealant kit of any of items 38 to 44 for use in soft tissue procedures and/or internal wound procedures.

[46] The fibrin sealant of any of items 27 to 37, or the fibrin sealant kit of any of items 38 to 44 for use as a hemostat, a tissue sealant, or a wound adhesive.

[47] Use of the fibrin sealant of any of items 27 to 37, or the fibrin sealant kit of any of items 38 to 44, in soft tissue procedures and/or internal wound procedures.

[48] Use of an isolated recombinant fibrinogen comprising
i. fibrinogen alpha-chains, preferably each comprising, or consisting of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, identity with any of SEQ ID NOs: 6, 7, 8, 41, 42, 43, 45, 46, 47, 48, 49, and 50, preferably with SEQ ID NO: 6, or with SEQ ID NO: 7, or with SEQ ID NO: 8, or with SEQ ID NO: 41, or with SEQ ID NO: 42, or with SEQ ID NO: 43, preferably with SEQ ID NO: 6, SEQ ID NO: 7, or SEQ ID NO: 8; and/or
ii. fibrinogen beta-chains, preferably each comprising, or consisting of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, identity with SEQ ID NO: 9, or with SEQ ID NO: 44, preferably with SEQ ID NO: 9; and/or
iii. fibrinogen gamma-chains, preferably each comprising, or consisting of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, identity with SEQ ID NO: 10, or with SEQ ID NO: 11;
   characterized in that
(a) in one, preferably both, of the fibrinogen alpha-chains, one or more lysine (K) and/or arginine (R) residues selected from the group consisting R38, R42, K97, K100, R114, R123, R129, R135, K210, R216, R218, K225, K227, K238, K243, K249, R258, R271, R287, R308, R353, R367, R425, R426, K432, K437, K440, R443, K446, K448, R458, R459, K463, K467, K476, K480, R510, R512, K527, R547, K558, R573, K575, K581, R591, K599, K602, K620, R621, K625, R687 and R847 are substituted or deleted; and/or
(b) in one, preferably both, of the fibrinogen beta-chains, one or more lysine (K) and/or arginine (R) residues selected from the group consisting of R44, K51, K52, R53, R60, R72, K77, K83, R87, K152, R158, K160, K163, K178, R285, K328, R334, K348, K353, K367, K374, K458 and K471 are substituted or deleted; and/or
(c) in one, preferably both, of the fibrinogen gamma-chains, one or more lysine (K) and/or arginine (R) residues selected from the group consisting of R40, K79, K84, K88, K111, K114, K185, K199, K238, K292, R301, K382 and K399 are substituted or deleted;
   in a fibrin sealant, or a fibrin sealant kit.

[49] A eucaryotic cell, comprising exogenous nucleotide sequences encoding one or more recombinant fibrinogen alpha-chains, one or more recombinant fibrinogen beta-chains, and one or more recombinant fibrinogen gamma-chains; wherein
i. preferably each of the, one or more fibrinogen alpha-chains comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.7%, identity with any of SEQ ID NOs: 6, 7, 8, 41, 42, 43, 45, 46, 47, 48, 49, and 50, preferably with SEQ ID NO: 6, or with SEQ ID NO: 7, or with SEQ ID NO: 8, or with SEQ ID NO: 41, or with SEQ ID NO: 42, or with SEQ ID NO: 43, more preferably with SEQ ID NO: 6, SEQ ID NO: 7, or SEQ ID NO: 8; and/or
ii. preferably each of the, one or more fibrinogen beta-chains comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.7%, identity with SEQ ID NO: 9, or with SEQ ID NO: 44, preferably with SEQ ID NO: 9; and/or
iii. preferably each of the, one or more fibrinogen gamma-chains comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.7%, identity with SEQ ID NO: 10, or with SEQ ID NO: 11;
   characterized in that
(a) in one, preferably both, of the fibrinogen alpha-chains, with reference to the respective positions in SEQ ID NO: 2, one or more lysine (K) and/or arginine (R) residues selected from the group consisting R38, R42, K97, K100, R114, R123, R129, R135, K210, R216, R218, K225, K227, K238, K243, K249, R258, R271, R287, R308, R353, R367, R425, R426, K432, K437, K440, R443, K446, K448, R458, R459, K463, K467, K476, K480, R510, R512, K527, R547, K558, R573, K575, K581 R591, K599, K602, K620, R621, K625, R687 and R847, are substituted or deleted; and/or
(b) in one, preferably both, of the fibrinogen beta-chains, with reference to the respective positions in SEQ ID NO: 3, one or more lysine (K) and/or arginine (R) residues selected from the group consisting of R44, K51, K52, R53, R60, R72, K77, K83, R87, K152, R158, K160, K163, K178, R285, K328, R334, K348, K353, K367, K374, K458 and K471 are substituted or deleted; and/or
(c) in one, preferably both, of the fibrinogen gamma-chains, with reference to the respective positions in SEQ ID NO: 4, one or more lysine (K) and/or arginine (R) residues selected from the group consisting of R40, K79, K84, K88, K111, K114, K185, K199, K238, K292, R301, K382 and K399 are substituted or deleted.

[50] The eucaryotic cell of item 49, which, when cultured under conditions wherein the fibrinogen is produced, produces, preferably functional, recombinant fibrinogen.

[51] The eucaryotic cell according to item 49 or 50, which is a mammalian cell culture cell, preferably a Chinese hamster ovary (CHO) cell, preferably selected from a CHO-K1, a CHO-DG44, a CHO-Pro minus and a CHO-S cell, a PER.C6 cell, or a human embryonic kidney (HEK) cell, preferably selected from a HEK 293, a HEK 293T, a HEK 293S and a HEK 293 EBNA cell, or a murine myeloma cell, preferably selected from a NS0 cell, a NS-1 cell and a Sp2/0 cell, or a Baby hamster kidney (BHK) cell, preferably a BHK-21 cell, or a rat myeloma cell, preferably a YB2/0 cell or a YB2/3HL cell.

[52] The fibrin sealant of any of items 27 to 37, or the fibrin sealant kit of any of items 38 to 44, or the fibrin sealant or fibrin sealant kit for use according to item 45 or 46, or the use according to item 47 or 48, or the eucaryotic cell according to any of items 49-51, wherein each fibrinogen alpha-chain further comprises one or more (for example 1, 2, 3 or 4) substitutions or deletions inside 4, or 3, or 2, amino acids directly up- and/or downstream of at least one (e.g. 1, or 2, or 3, etc.) metalloproteinase (MMP) cleavage site (e.g. the at least one MMP cleavage site, with reference to the respective positions in SEQ ID NO: 2, is at K432); preferably wherein the one or more substitutions or deletions inside 4, or 3, or 2, amino acids directly up- and/or downstream of at least one metalloproteinase (MMP) cleavage site in the fibrinogen alpha-chains, with reference to the respective positions in SEQ ID NO: 2, comprise, or consist of, substitution of L433 and/or T435, preferably L433H and/or T435R.

[53] A method for the production of isolated recombinant fibrinogen, preferably carrying one or more site-directed mutations relative to human wild-type fibrinogen, comprising the steps of
A) culturing eucaryotic cells comprising exogenous nucleotide sequences encoding at least one fibrinogen alpha-chain, at least one fibrinogen beta-chain, and at least one fibrinogen gamma-chain, preferably derived from mammalian, more preferably human, fibrinogen, in a culture medium under conditions wherein fibrinogen is produced; and
B) recovering the recombinant fibrinogen produced;
   wherein step A) is carried out in the presence of at least one matrix metalloproteinase (MMP) inhibitor, preferably selected from the group consisting of 1,10 Phenanthroline, UK 370106, GM6001, and a combination thereof, preferably UK 370106.

[54] A method for the production of isolated recombinant fibrinogen, preferably carrying one or more site-directed mutations relative to human wild-type fibrinogen, comprising the steps of
A) culturing eucaryotic cells comprising exogenous nucleotide sequences encoding at least one fibrinogen alpha-chain, at least one fibrinogen beta-chain, and at least one fibrinogen gamma-chain, preferably derived from mammalian, more preferably human, fibrinogen, in a culture medium under conditions wherein fibrinogen is produced; and
B) recovering the recombinant fibrinogen produced;
   wherein each fibrinogen alpha-chain comprises one or more (for example 1, 2, 3 or 4) substitutions or deletions inside 4, or 3, or 2, amino acids directly up- and/or downstream of at least one (e.g. 1, or 2, or 3, etc.) metalloproteinase (MMP) cleavage site (e.g. the at least one MMP cleavage site, with reference to the respective positions in SEQ ID NO: 2, is at K432); preferably wherein the one or more substitutions or deletions inside 4, or 3, or 2, amino acids directly up- and/or downstream of at least one metalloproteinase (MMP) cleavage site in the fibrinogen alpha-chains, with reference to the respective positions in SEQ ID NO: 2, comprise, or consist of, substitution of L433 and/or T435, preferably L433H and/or T435R.

[55] The method for the production of isolated recombinant fibrinogen of item 53 or 54, wherein step B) comprises collecting at least a portion of the culture medium, preferably containing greater than 0.5 μg/ml, more preferably greater than 1 μg/ml, even more preferably greater than 2 μg/ml, even more preferably greater than 5 μg/ml, even more preferably greater than 10 μg/ml, even more preferably greater than 20 μg/ml, e.g. greater than 50 μg/ml, or e.g. greater than 100 μg/ml, recombinant fibrinogen.

[56] The method for the production of isolated recombinant fibrinogen of any of items 53 to 55, wherein step B) comprises, optionally concentrating the fibrinogen from the culture medium to form a concentrated medium and, purifying the recombinant fibrinogen, preferably from the culture medium.

[57] The method for the production of isolated recombinant fibrinogen of item 56, wherein the purifying is carried out by chromatographic methods, preferably affinity chromatography.

[58] The method for the production of isolated recombinant fibrinogen according to any of items 53 to 57, wherein the eucaryotic cells are mammalian cell culture cells, preferably Chinese hamster ovary (CHO) cells, preferably selected from CHO-K1, CHO-DG44, CHO-Pro minus and CHO-S cells, PER.C6 cells, or human embryonic kidney (HEK) cells, preferably selected from HEK 293, HEK 293T, HEK 293S or HEK 293 EBNA cells, or murine myeloma cells, preferably selected from NS0 cells, NS-1 cells and Sp2/0 cells, or Baby hamster kidney (BHK) cells, preferably BHK-21 cells, or rat myeloma cells, preferably YB2/0 cells or YB2/3HL cells.

[59] The method for the production of isolated recombinant fibrinogen according to any of items 53 to 58, or the eucaryotic cell according to any of items 47 to 49, wherein at least one, preferably each, of the exogenous nucleotide sequences is optimized for expression in the mammalian cell culture cells under conditions wherein the fibrinogen is produced.

[60] The method for the production of isolated recombinant fibrinogen according any of items 53 to 59, or the eucaryotic cell according to any of items 47 to 49, wherein the cells comprise a single nucleotide construct comprising the nucleotide sequences encoding the fibrinogen alpha-, beta- and gamma-chains.

[61] The method for the production of isolated recombinant fibrinogen according to any of items 53 to 60, wherein the isolated recombinant fibrinogen is the isolated recombinant fibrinogen according to any of items 1 to 26.

[62] The method for the production of isolated recombinant fibrinogen according to any of items 53 to 60, wherein
i. preferably each of the one or more fibrinogen alpha-chains comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, amino acid sequence identity with any of SEQ ID NOs: 6, 7, 8, 41, 42, 43, 45, 46, 47, 48, 49, and 50, preferably with SEQ ID NO: 6, or with SEQ ID NO: 7, or with SEQ ID NO: 8, or with SEQ ID NO: 41, or with SEQ ID NO: 42, or with SEQ ID NO: 43, more preferably with SEQ ID NO: 6, SEQ ID NO: 7, or SEQ ID NO: 8; and/or
ii. preferably each of the one or more fibrinogen beta-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, amino acid sequence identity with SEQ ID NO: 9, or with SEQ ID NO: 44, preferably with SEQ ID NO: 9; and/or
iii. preferably each of the one or more fibrinogen gamma-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, amino acid sequence identity with SEQ ID NO: 10, or with SEQ ID NO: 11;
   characterized in that
(a) in one, preferably both, of the fibrinogen alpha-chains, with reference to the respective positions in SEQ ID NO: 2, one or more lysine (K) and/or arginine (R) residues selected from the group consisting R38, R42, K97, K100, R114, R123, R129, R135, K210, R216, R218, K225, K227, K238, K243, K249, R258, R271, R287, R308, R353, R367, R425, R426, K432, K437, K440, R443, K446, K448, R458, R459, K463, K467, K476, K480, R510, R512, K527, R547, K558, R573, K575, K581, R591, K599, K602, K620, R621, K625, R687 and R847 are substituted or deleted; and/or
(b) in one, preferably both, of the fibrinogen beta-chains, with reference to the respective positions in SEQ ID NO: 3, one or more lysine (K) and/or arginine (R) residues selected from the group consisting of R44, K51, K52, R53, R60, R72, K77, K83, R87, K152, R158, K160, K163, K178, R285, K328, R334, K348, K353, K367, K374, K458 and K471 are substituted or deleted; and/or
(c) in one, preferably both, of the fibrinogen gamma-chains, with reference to the respective positions in SEQ ID NO: 4, one or more lysine (K) and/or arginine (R) residues selected from the group consisting of R40, K79, K84, K88, K111, K114, K185, K199, K238, K292, R301, K382 and K399 are substituted or deleted.

[63] A method for identifying plasmin resistant recombinant fibrinogen variants comprising the steps of
A) Identification of plasmin cleavage sites in fibrinogen, by
   i) incubating, preferably by covering, a fibrin gel, of preferably 10-30 mg/ml, more preferably 20 mg/ml density, with a plasmin solution of preferably 0.001 to 0.01 mU/ml;
   ii) collecting supernatant at different timepoints after addition of the plasmin solution, and optionally inactivating the plasmin, preferably by addition of PSMF and/or heat;
   iii) determining cleavage sites in fibrinogen, preferably using liquid chromatography mass spectrometry or solid phase extraction (SPE) mass spectrometry, more preferably using high performance liquid chromatography/electrospray ionization mass spectrometry (HPLC/ESI-MS), to obtain a time-resolved cleavage pattern of fibrinogen; and
iv) identifying lysine (K) and/or arginine (R) residues immediately, and optionally immediately after, the cleavage sites in the amino acid sequence of fibrinogen; and
B) optionally selecting one or more of the identified lysine (K) and/or arginine (R) residues in the amino acid sequence of fibrinogen, preferably based on the time-resolved cleavage pattern, for substitution or deletion, to obtain a new recombinant fibrinogen variant; and optionally incubating the new recombinant fibrinogen variant with plasmin to test for sensitivity to proteolytic cleavage by plasmin.

[64] The method of item 63, wherein in step A) iii) an internal standard, preferably angiotensin II, is used for analysing the cleavage pattern.

[65] The method of item 63 or 64, wherein in step iii) the collected supernatant is purified using liquid chromatography, preferably HPLC, or solid phase extraction (SPE).

[66] The method of any of items 63 to 65, wherein the fibrinogen is wild-type fibrinogen.

[67] The method of any of items 63 to 65, wherein the fibrinogen is a mutant fibrinogen, preferably selected from of the isolated recombinant fibrinogens according to any of items 1 to 26.

### BRIEF DESCRIPTION OF THE SEVERAL VIEWS OF THE DRAWINGS

FIG. 1: Schematic representation of the modification of fibrinogen. (A) Structure of fibrinogen with labeled target amino acid positions in the 3D structure (PDB: 3GHG5). (B) Amino acid sequence of the plasmin target sites, the catalytic residue (light grey) and the modified amino acids of the mutants (dark grey).

FIG. 2: Map of the fibrinogen expression plasmid pcDNA4-TO-fbg. The plasmid comprises the genes for α-, β- and γ-chain of fibrinogen, each controlled by the human cytomegalovirus (CMV) promoter. Bovine growth hormone (bGH) polyadenylation signal and transcription termination sequences were added to each gene cassette.

FIG. 3: Analysis of rFbgi variants. (A) Exemplary size exclusion chromatography chromatogram of His-rFbgi. (B) SDS-PAGE of Fbg control (1), His-rFbgi (2), Ala-rFbgi (3) under non-reduced conditions and Fbg control (4), His-rFbgi (5), Ala-rFbgi (6) under reduced conditions.

FIG. 4: Improvement of rFbg expression. (A) Cutout (AA397-440) of MS-analysis of in gel digest of the truncated α-chain (Full analysis FIG. 17). (B-C) Predicted structure of the fibrinogen α-chain by AlphaFold (P02671), with the target AA K432 highlighted as zigzag line, aligned with the crystal structure of fibrinogen (PDB: 3GHG). (D) SDS-PAGE of the screening of different protease inhibitors on the degradation of Fbg in CHO cell culture supernatant after 2 days of incubation; 1: Fbg control in Tris-buffer, 2: Fbg, 3: Fbg+EDTA; 4: Fbg+1,10 Phenantroline, 5: Fbg+GM6001, 6: Fbg+UK 370106. (E) SDS-PAGE of His-rFbg432 with the addition of UK 370106 during expression; 1: Fbg control reduced, 2: His-rFbg432 reduced, 3: Fbg control non-reduced, 4: His-rFbg432 non-reduced.

FIG. 5: Plasmin cleavage analysis of rFbg variants. (A) Structure of fibrinogen with the target AAs positions in the 3D structure (PDB: 3GHG5) marked with lines. (B) Plasmin cleaves target site in Fbg. Analysis of plasmin cleavage of target sites in WT-Fbg (C), His-rFbg (D) and Ala-rFbg (E). The presented cleavage sites are C-terminal to K. P1 of targeted cleavage sites are marked as light grey characters. Peptide fragments detected after plasmin (dark grey) and after additional ProAlanase cleavage (light grey) are shown. Position 382 shows a partial cleavage for the mutants (both cleaved and non-cleaved peptides detected). Position 399 could only be detected in His-rFbg and WT-Fbg.

FIG. 6: Temporal analysis of the plasmin cleavage sites of fibrin gels. The signal intensity of the identified peptides was normalized to the standard angiotensin II, after that an intensity of all detected peptides either starting (A, B, C) or ending (D, E, F) at the indicated position were summed up. (A, D) Analysis of the cleavage sites of the α-chain over time by signals from peptides starting (A) or ending (D) with the indicated position. (B) Analysis of the cleavage sites of the β-chain over time by signals from peptides starting with the indicated position. (E) Analysis of the cleavage sites of the β-chain over time by signals from peptides ending with the indicated position. (C) Analysis of the cleavage sites of the γ-chain over time by signals from peptides starting with the indicated position. (F) Analysis of the cleavage sites of the γ-chain over time by signals from peptides ending with the indicated position.

FIG. 7: Sequence of the fibrinogen alpha-chains isoforms 2 (alpha) and 1 (alpha-E) with cleavage and mutations sites highlighted with rectangles of different patterns.

FIG. 8: Sequence of the fibrinogen beta- and gamma-chains with cleavage and mutations sites highlighted with rectangles of different patterns.

FIG. 9: Construction of fibrinogen expression plasmid.

FIG. 10: (A)-(B): Analysis of recombinant fibrinogen expression in CHO cells. (A) SDS-PAGE (5-12%) WT-rFbgi after the first purification using AiEx; (B) Ser-rFbgi after the first purification using AiEx. (C)-(E): SDS-PAGE analysis of the expression of rFbg, His-rFbg and Ala-rFbg: (C) SDS-Page of WT rFbg: M: Marker; 1: Fbg non-reduced; 2: rFbg non-reduced; 3: Fbg reduced; 4: rFbg reduced. (D) SDS-PAGE of rFbg variants: M: Marker; 1: His-rFbg non-reduced; 2: Ala-rFbg non-reduced. (E) SDS-PAGE of rFbg variants: M: Marker; 1: His-rFbg reduced; 2: Ala-rFbg reduced.

FIG. 11: NanoLC-MS/MS analysis of WT-Fbg, His- and Ala-rFbg γ-chain after plasmin digest ((A) and (B) correspond to aa1-200 and aa201-453 in the γ-chain isoform γ′). Suggested modifications are highlighted in light grey (WT-Fbg and His-rFbg) or dark grey (Ala-rFbg).

FIG. 12: NanoLC-MS/MS analysis of WT-Fbg, His- and Ala-rFbg γ-chain after plasmin and subsequent ProAla digest. Suggested modifications are highlighted in light grey (His- and Ala-rFbg) or dark grey (WT-Fbg).

FIG. 13: NanoLC-MS/MS analysis of WT-Fbg, His- and Ala-rFbg α-chain after plasmin digest. Suggested modifications are highlighted in light grey (WT-Fbg and His-rFbg) or dark grey (Ala-rFbg).

FIG. 14: NanoLC-MS/MS analysis of WT-Fbg, His- and Ala-rFbg α-chain after plasmin and subsequent ProAla digest. Suggested modifications are highlighted in light grey (Ala- and His-rFbg) or dark grey (WT-Fbg).

FIG. 15: NanoLC-MS/MS analysis of WT-Fbg, His- and Ala-rFbg β-chain after plasmin digest. Suggested modifications are highlighted in light grey (WT-Fbg and His-rFbg) or dark grey (Ala-rFbg).

FIG. 16: NanoLC-MS/MS analysis of WT-Fbg, His- and Ala-rFbg β-chain after plasmin and subsequent ProAla digest. Suggested modifications are highlighted in light grey (His-rFbg and Ala-rFbg) or dark grey (WT-Fbg).

FIG. 17: NanoLC-MS/MS analysis of the in-gel digest of His-rFbg α-chain.

FIG. 18: (A): SDS-PAGE analysis of the expression of His-rFbg432 and His-rFbg5xKtoH. M: Marker; 1: His-rFbg432 non-reduced. 2: His-rFbg432 reduced. 3: His-rFbg5xKtoH non-reduced. 4: His-rFbg5xKtoH reduced. (B)-(C): Screening of different inhibitors for the expression of rFbg. (B) SDS-PAGE of Fbg incubated with ExpiCHO-S™ cell supernatant and different inhibitors after 2 h: M: Marker; 1: Fbg 2: Fbg+PMSF 3: Fbg+Aprotinin; 4: Fbg+EDTA; 5: Fbg+1,10 Phenanthroline; 6: Fbg+GM6001; 7: Fbg+UK 370106; 8: Fbg control. (C) SDS-PAGE of Fbg incubated with ExpiCHO-S cell supernatant and different inhibitors after 14 h: M: Marker; 1: Fbg 2: Fbg+PMSF 3: Fbg+Aprotinin; 4: Fbg+EDTA; 5: Fbg+1,10 Phenanthroline; 6: Fbg+GM6001; 7: Fbg+UK 370106; 8: Fbg control.

FIG. 19: NanoLC-MS/MS analysis of the in-gel digest of His-rFbgK432 α-chain.

FIG. 20: NanoLC-MS/MS analysis of (A) His- and (B) Ala-rFbg γ-chain after plasmin digest.

FIG. 21: Schematic representation of the fibrinogen modifications in the α-chain with the positions of the target amino acids (dark) in the 3D structure (AlphaFold, AF-P02671-F1). Amino acid sequence of the MMP target sites (light gray) and the modified amino acids of the mutants (dark gray).

FIG. 22: SDS-PAGE analysis of the expression of His-rFbgH433R435. 1: His-rFbgH433R435 reduced. 2: His-rFbgH433R435 non-reduced.

FIG. 23: NanoLC-MS/MS analysis of the in-gel digest of His-rFbgH433R435 α-chain.

FIG. 24: NanoLC-MS/MS analysis of the in-gel digest of His-rFbgH433R435 β-chain.

FIG. 25: NanoLC-MS/MS analysis of the in-gel digest of His-rFbgH433R435 γ-chain.

FIG. 26: Thrombin-catalyzed fibrin polymerization. Polymerization of fibrinogen (0.1 mg/mL) was initiated by the addition of thrombin (0.05 U/mL), factor XIIIa (Fibrogammin®, CSL Behring) (50 mU/ml) as well as CaCl₂ (10 mM). The change in turbidity at 350 nm was followed with time (0-3600 sec). Representative polymerization curves of plasma Fibrinogen (square) and His-rFbgH433R435 (triangle).

FIG. 27: FXIIIa-catalyzed cross-linking of fibrinogen. Crosslinking of His-rFbgH433R435 by rFXIIIa was examined with SDS-PAGE, under reducing conditions. Fibrinogen (0.1 mg/mL) was mixed with FXIIIa (0.5 mU/mL) and thrombin (0.05 U/mL), and the reaction was incubated for the specified time at 37° C. The reduced fibrin chains (α, β, γ, cross-linked γ-γ dimer) are indicated on the right side of the gel.

### DESCRIPTION OF SPECIFIC EMBODIMENTS OF THE INVENTION

The present invention relates to new isolated recombinant fibrinogen variants with improved stability in the presence of plasmin, a fibrin sealant and a fibrin sealant kit comprising said new isolated recombinant fibrinogen variants, and eucaryotic cells expressing said new recombinant fibrinogen variants. The present invention further relates to a method for the production of isolated recombinant fibrinogen variants using cell culture cells, and a method for identifying plasmin-resistant recombinant fibrinogen variants.

In a first aspect, the present invention is directed to an isolated recombinant fibrinogen comprising two fibrinogen alpha-chains, two fibrinogen beta-chains and two fibrinogen gamma-chains, having amino acid sequences derived from human fibrinogen.

The term “fibrinogen” is known to the person skilled and as used herein refers to a hexameric protein comprising two fibrinogen alpha-chains, two fibrinogen beta-chains and two fibrinogen gamma-chains. The terms “recombinant fibrinogen” and “isolated recombinant fibrinogen” are also known to the skilled person and refer to fibrinogen produced by genetic engineering of cells, preferably using expression constructs encoding fibrinogen chains and/or fibrinogen chain precursors. Methods for the production of recombinant fibrinogen are for example described in the following patents and patent applications: WO9607728 A1, WO2018161135 A1 U.S. Pat. Nos. 6,037,457 A, 6,083,902 A, US 2010151522 A1, US2010159512 A1, US2017037108 A1 and U.S. Ser. No. 10/208,101 B2.

The term “fibrinogen alpha-chain”, as known to the skilled person, refers to one of the three subunits of hexameric fibrinogen protein. As described above, there are two human wild-type isoforms of fibrinogen alpha-chain; the isoform 2 which is in general referred to as α-chain, and which herein is referred to as “isoform alpha” or “isoform α”; and the isoform E (αE), also known as isoform 1, which herein is referred to as “isoform alpha-E” or “isoform αE”. The full-length human wild-type fibrinogen alpha-chain isoform alpha before post-translational modification (PTM) has for example an amino acid sequence as shown in SEQ ID NO: 1 (see also Table 1). The full-length human wild-type fibrinogen alpha-chain isoform alpha-E before post-translational modification has for example an amino acid sequence as shown in SEQ ID NO: 2 (see also Table 1).

The term “Aα” or “A alpha” (having a wild-type sequence as for example shown in SEQ ID NO: 6) refers to the secreted form of alpha-chain isoform alpha, which lacks the N-terminal, for example 19 amino acid long, “signal peptide” or “signal sequence” present in the full-length alpha-chain before post-translational modification. The Aa form still includes the “Fibrinopeptide A”, which consists for example of amino acids 20-35 in the full-length amino acid sequence of isoform alpha before post-translational modification. Similarly, the term “AαE” or “A alpha-E” (having a wild-type sequence as for example shown in SEQ ID NO: 8) refers to the secreted form of alpha-chain isoform alpha-E, which lacks the N-terminal, for example 19 amino acid long, “signal peptide” or “signal sequence” present in the full-length amino acid sequence of isoform alpha-E before post-translational modification, but still includes the “Fibrinopeptide A” or (FpA), which consists for example of amino acids 20-35 of the full-length amino acid sequence of isoform alpha-E before post-translational modification.

The alpha-chain isoform alpha, although being synthesized as a, for example for the wild-type peptide 625 amino acid, precursor (i.e. A alpha), is present in plasma as an about 610 amino acids long polypeptide (having a wild-type sequence as for example shown in SEQ ID NO: 7), which misses at the C-terminus a number of amino acid residues compared to full-length isoform alpha and Aa just after secretion (see also Table 1).

The term “fibrinogen beta-chain”, as known to the skilled person, refers to the second of the three subunits of hexameric fibrinogen protein. The fibrinogen beta-chain is herein generally referred to as “beta-chain” or “β-chain”. In its full-length human wild-type fibrinogen beta-chain before post-translational modification has for example an amino acid sequence as shown in SEQ ID NO: 3 (see also Table 1). The term “Bβ” or “B beta” (having a wild-type sequence as for example shown in SEQ ID NO: 9) refers to the secreted form of the beta-chain after post-translational modification, which lacks the N-terminal, for example 30 amino acid long, “signal peptide” or “signal sequence” present in the full-length beta-chain before post-translational modification. The Bp form still includes the “Fibrinopeptide B”, which consists in wild-type Bp for example of amino acids 31-44 of the full-length amino acid sequence of fibrinogen beta-chain before post-translational modification.

The term “fibrinogen gamma-chain”, as known to the skilled person, refers to the third of the three subunits of hexameric fibrinogen protein. As described above, there are two human wild-type isoforms of fibrinogen gamma-chain. The first isoform is known as gamma or gamma A (γ or γA; also simply referred to as gamma-chain, or gamma A chain), and herein is referred to as gamma-chain “isoform gamma” or “isoform γ”. The second isoform is known as gamma′ or gamma B (γ′ or gamma; also simply referred to as gamma′-chain, or gamma B chain), and is herein referred to as gamma-chain “isoform gamma” or “isoform γ”. The full-length human wild-type fibrinogen gamma-chain isoform gamma before post-translational modification has for example an amino acid sequence as shown in SEQ ID NO: 4 (see also Table 1). The full-length human wild-type fibrinogen gamma-chain isoform gamma′ before post-translational modification has for example an amino acid sequence as shown in SEQ ID NO: 5 (see also Table 1). The gamma-chain isoform gamma (having a wild-type sequence as for example shown in SEQ ID NO: 10), as well as the gamma-chain isoform gamma′ (having a wild-type sequence as for example shown in SEQ ID NO: 11), after post-translational modification, in their secreted form lack the N-terminal, for example 26 amino acid long, “signal peptide” or “signal sequence” present in the full-length gamma-chain before post-translational modification.

The fibrinogen alpha-, beta- and gamma-chains of the inventive isolated recombinant fibrinogen have amino acid sequences derived from human fibrinogen. This means that the fibrinogen amino acid sequences originate (before any molecular engineering) from human, preferably wild-type, amino acid sequences of fibrinogen, as for example shown in SEQ ID NOs: 1-11 (see also Table 1 below). One or several of the amino acid sequences of the individual peptides of the hexameric isolated recombinant fibrinogen may be as shown in SEQ ID NOs: 1-11, as long as at least one of the peptides of the isolated recombinant fibrinogen comprises one or more modifications as set out in detail below. Moreover, although these sequences are derived from human sequences, they may have been modified (beyond any modification in the lysine (K) and/or arginine (r) residues described below) e.g. by substitution, deletion, insertion, or post-translational modifications.

Additionally, it is to be understood that the wild-type sequences of the fibrinogen alpha-, beta- and gamma-chains are also subject to sequence variations, resulting in differences in the amino acid sequence. As such, even those fibrinogen alpha-, beta- and/or gamma-chains of the inventive isolated recombinant fibrinogen, not comprising one or more substitutions or deletions in one or more lysine (K) and/or arginine (R) residues in accordance with the present invention as set out below, may comprise sequence variations involving one or several amino acids, resulting in a difference to the amino acid sequences of SEQ ID NOs: 1-11.

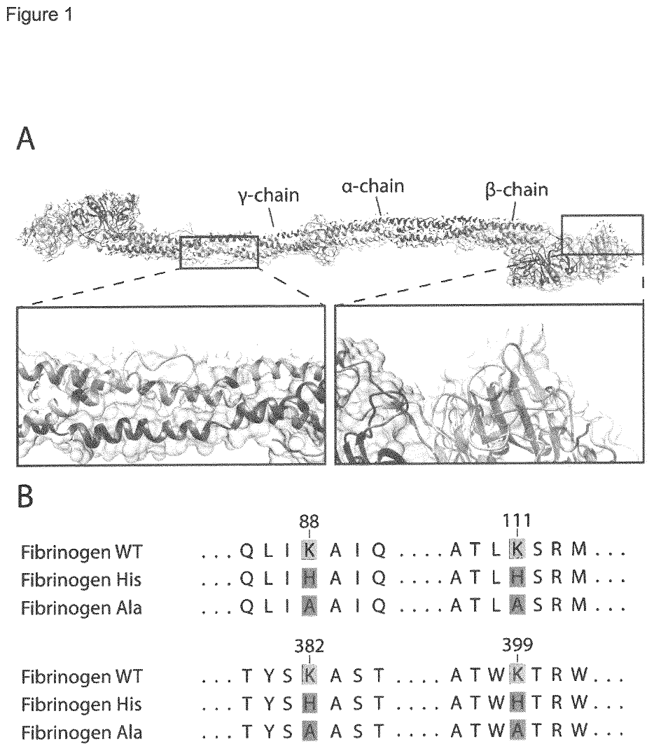

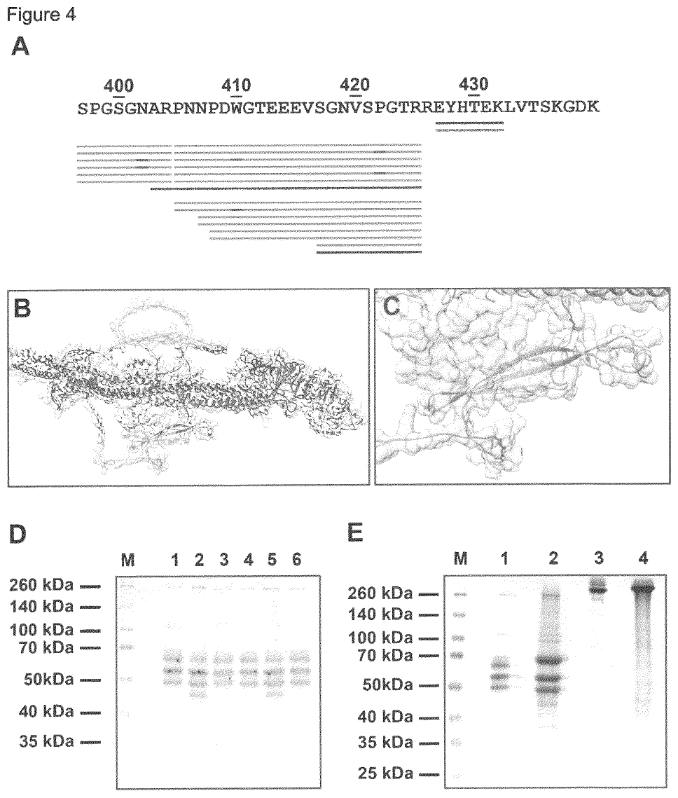

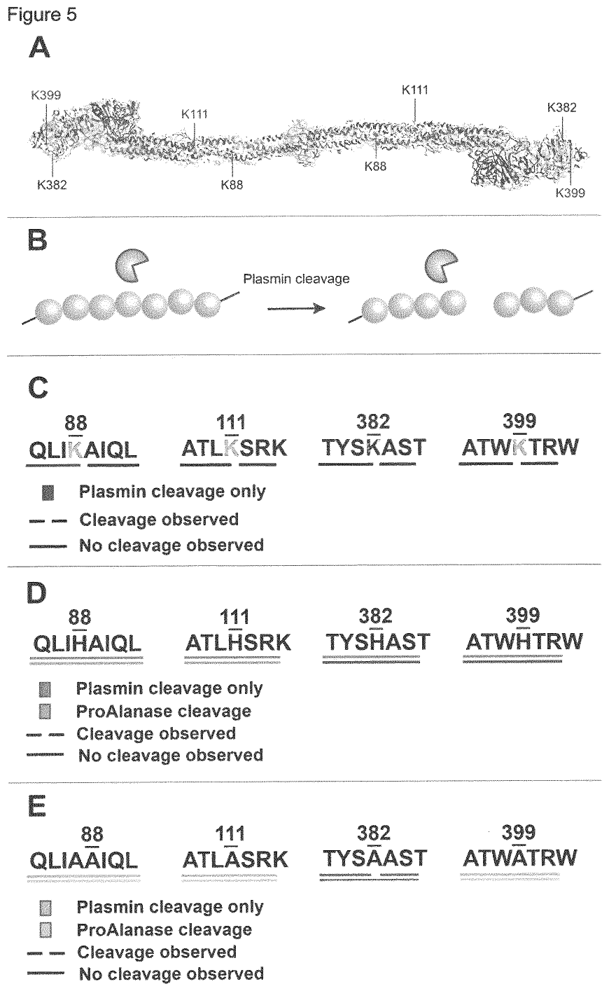

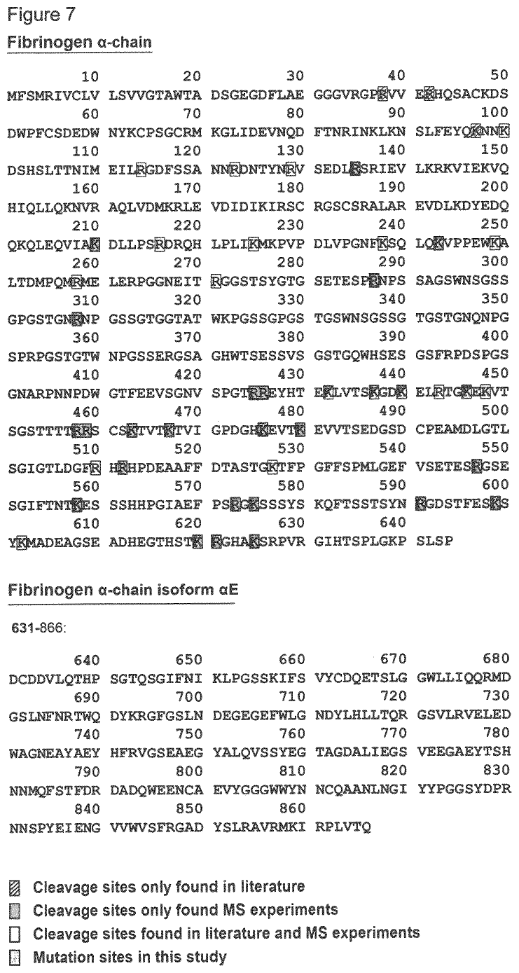

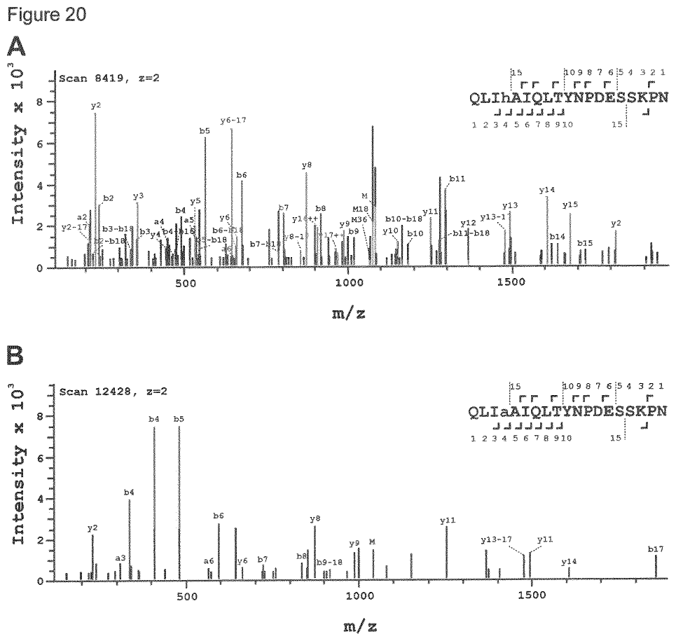

TABLE 1
List of Sequences

| SEQ ID NO: | Sequence Name | Sequence description |
|---|---|---|
| 1 | human fibrinogen alpha- | aa 1-19: signal peptide; aa20-35 Fibrinopeptide A; |
| | chain isoform alpha (α; | aa 36-644: amino acid chain sequence of fibrinogen |
| | also known as “isoform | alpha-chain; aa 630-aa 644: amino acids cleaved off in |
| | 2”; uniport ID P02671-2) | circulation |
| 2 | human fibrinogen alpha- | aa 1-19: signal peptide; aa 20-35 Fibrinopeptide A; |
| | chain isoform alpha-E | aa 36-866: amino acid chain sequence of fibrinogen |
| | (αE; also known as | alpha-chain isoform αE; alpha-chain isoform αE shares |
| | “isoform 1” uniport ID | with alpha chain aa 1-630 |
| | P02671-1) | |
| 3 | human fibrinogen beta- | aa 1-30: signal peptide; aa 31-44 Fibrinopeptide B; |
| | chain (β; uniport ID | aa 45-491: amino acid chain sequence of fibrinogen |
| | P02675) | beta-chain; |
| 4 | human fibrinogen | aa 1-26: signal peptide; aa 27-437: amino acid chain |
| | gamma-chain isoform | sequence of fibrinogen gamma-chain |
| | gamma (γ; also known | |
| | as “isoform Gamma-A” | |
| | or “γA”; uniport ID | |
| | P02679-2) | |
| 5 | human fibrinogen | aa 1-26: signal peptide; aa 27-453: amino acid chain |
| | gamma-chain isoform | sequence of fibrinogen gamma-chain isoform γ′ |
| | gamma′ (γ′; also known | |
| | as isoform “Gamma-B” | |
| | or “γB”; uniport ID | |
| | P02679-1) | |
| 6 | human fibrinogen alpha- | secreted form of alpha-chain a w/o signal peptide, |
| | chain α w/o signal | corresponding to aa 20-644 of SEQ ID NO: 1 |
| | peptide, also known as | |
| | Aα | |
| 7 | truncated fibrinogen | truncated fibrinogen alpha-chain a of 610 aa as found |
| | alpha-chain α of 610 aa | in circulation (aa 20 to 629 of SEQ ID NO: 1) |
| 8 | human fibrinogen alpha- | secreted form of alpha-chain aE w/o signal peptide, |
| | chain αE w/o signal | corresponding to aa 20-866 of SEQ ID NO: 2 |
| | peptide; also known as | |
| | “AαE” | |
| 9 | human fibrinogen beta- | secreted form of beta-chain w/o signal peptide, |
| | chain w/o signal peptide; | corresponding to aa 31- 491 of SEQ ID NO: 3 |
| | also known as “Bβ” | |
| 10 | human fibrinogen | secreted form of gamma-chain γ w/o signal peptide, |
| | gamma-chain γ w/o | corresponding to aa 27-437 of SEQ ID NO: 4 |
| | signal peptide; | |
| 11 | human fibrinogen | secreted form of gamma-chain γ′ w/o signal peptide, |
| | gamma-chain γ′ w/o | corresponding to aa 27-453 of SEQ ID NO: 5 |
| | signal peptide | |
| 12 | FGG-Lys88-S-FW | 5′GCAGCTGATC AGCGCCATCC AGCTGACCTA CAACCCCGAC GAGG3′ |
| 13 | FGG-Lys88-S-RV | 5′GGTCAGCTGG ATGGCGCTGA TCAGCTGCTT CACTTCGCTG GTC3′ |
| 14 | FGG-Lys111-S-FW | 5′CCGCCACCCT GAGCTCCCGG AAGATGCTGG AAGAGATC3′ |
| 15 | FGG-Lys111-S-RV | 5′CGGGAGCTCA GGGTGGCGGC GTCGATCATG TTG3′ |
| 16 | FGG-Lys356-S-FW | 5′CTACAGCAGC GCCAGCACCC CCAACGGCTA CGACAAC3′ |
| 17 | FGG-Lys356-S-RV | 5′GTGCTGGCGC TGCTGTAGGT GCCGCCCTGG TAGTACACG3′ |
| 18 | FGG-Lys373-S-FW | 5′CACCTGGAGC ACCCGGTGGT ACAGCATGAA GAAAACCAC3′ |
| 19 | FGG-Lys373-S-RV | 5′CCGGGTGCTC CAGGTGGCCC AGATGATGCC GTTGT3′ |
| 20 | oligonucleotide #1 | TGGCGGCCGCTCGAGTCTAGGCCACCATGGTGTCCATGC |
| 21 | oligonucleotide #2 | TTGTTCGAAGGGCCCTCTAGCCTGAGTGACCAGGGGC |
| 22 | oligonucleotide #3 | GCCGCCAGTGTGCTGGAATTGCCACCATGGTCCGGATGG |
| 23 | oligonucleotide #4 | TGATGGATATCTGCAGAATTTCACTGCTGAGGAAAGAATGGCC |
| 24 | oligonucleotide #5 | GCCGCCAGTGTGCTGGAATTGCCACCATGGTCTGGTCCC |
| 25 | oligonucleotide #6 | TGATGGATATCTGCAGAATTTCACAGGTCGTCCTCGGGG |
| 26 | oligonucleotide #7 | GTATTGGGCGCTCTTCCGCTGTACGGGCCAGATATACGCG |
| 27 | oligonucleotide #8 | CGCGTATATCTGGCCCGTACCCATAGAGCCCACCGCATCC |
| 28 | oligonucleotide #9 | GGATGCGGTGGGCTCTATGGGTACGGGCCAGATATACGCG |
| 29 | oligonucleotide #10 | GAGTCAGTGAGCGAGGAAGCCCATAGAGCCCACCGCATCC |
| 30 | oligonucleotide #68 | TGTTCGAAGGGCCCTCTAGATCAAGCCTGAGTGACCAGGGGCC |
| 31 | oligonucleotide #198 | TTGTTCGAAGGGCCCTCTAGCTAGGGGGACAGGGAAG |
| 32 | oligonucleotide #201 | TAAGAAGGAGATATACCATGGAAGCAGCAGTCG |
| 33 | oligonucleotide #302 | CATCTGGTCACTTCTAAAGGAGATAAAGAGC |
| 34 | oligonucleotide #303 | CATCTGGTCACTTCTCATGGAGATCATGAGCTCAGGACTGGTC |
| | | ATGAGCATGTCACCTCTGGTAGCACAACC |
| 35 | oligonucleotide #304 | CCTTTAGAAGTGACCAGATGTTCTGTGTGGTACTCTC |
| 36 | oligonucleotide #305 | CCATGAGAAGTGACCAGATGTTCTGTGTGGTACTCTC |
| 37 | FGG Insert Fw | 5′-GAGAACAAGA CCAGCGAAGT GAA-3′ |
| 38 | FGG Insert Rv | 5′-CTTCATGGTG GTTTTCTTCA TGC-3′ |
| 39 | FGG Vector Fw | 5′-GTACAGCATG AAGAAAACCA CCA-3′ |
| 40 | FGG Vector Rv | 5′-CTTCACTTCG CTGGTCTTGT TCT-3′ |
| 41 | human fibrinogen alpha- | secreted form of alpha-chain α w/o signal peptide and |
| | chain α w/o signal | w/o FpA, corresponding to aa 36-644 of SEQ ID NO: 1 |
| | peptide and w/o FpA | |
| 42 | truncated fibrinogen | truncated fibrinogen alpha-chain α as found in |
| | alpha-chain α of 594 aa | circulation further lacking FpA (aa 36 to 629 of SEQ ID |
| | | NO: 1) |
| 43 | human fibrinogen alpha- | secreted form of alpha-chain αE w/o signal peptide |
| | chain αE w/o signal | and w/o FpA, corresponding to aa 36-866 of SEQ ID |
| | peptide and w/o FpA | NO: 2 |
| 44 | human fibrinogen beta- | secreted form of beta-chain w/o signal peptide and w/o |
| | chain w/o signal peptide | FpB, corresponding to aa 45-491 of SEQ ID NO: 3 |
| | and w/o FpB | |
| 45 | fibrinogen alpha-chain | |
| | isoform alpha aa 20-288 | |
| 46 | fibrinogen alpha-chain | |
| | isoform alpha aa 20-316 | |
| 47 | fibrinogen alpha-chain | |
| | isoform alpha aa 20-328 | |
| 48 | fibrinogen alpha-chain | |
| | isoform alpha aa 36-288 | |
| 49 | fibrinogen alpha-chain | |
| | isoform alpha aa 36-316 | |
| 50 | fibrinogen alpha-chain | |
| | isoform alpha aa 36-328 | |
| 51 | sequence shown in | |
| | FIG. 1B | |
| 52 | sequence shown in | |
| | FIG. 1B | |
| 53 | sequence shown in | |
| | FIG. 1B | |
| 54 | sequence shown in | |
| | FIG. 1B | |
| 55 | sequence shown in | |
| | FIG. 1B | |
| 56 | sequence shown in | |
| | FIG. 1B | |
| 57 | sequence shown in | |
| | FIG. 1B | |
| 58 | sequence shown in | |
| | FIG. 1B | |
| 59 | sequence shown in | |
| | FIG. 1B | |
| 60 | sequence shown in | |
| | FIG. 1B | |
| 61 | sequence shown in | |
| | FIG. 1B | |
| 62 | sequence shown in | |
| | FIG. 1B | |
| 63 | sequence shown in | |
| | FIG. 4A | |
| 64 | sequence shown in | |
| | FIG. 5C | |
| 65 | sequence shown in | |
| | FIG. 5C | |
| 66 | sequence shown in | |
| | FIG. 5C | |
| 67 | sequence shown in | |
| | FIG. 5C | |
| 68 | sequence shown in | |
| | FIG. 5D | |
| 69 | sequence shown in | |
| | FIG. 5D | |
| 70 | sequence shown in | |
| | FIG. 5D | |
| 71 | sequence shown in | |
| | FIG. 5D | |
| 72 | sequence 1 shown in | |
| | FIG. 5E | |
| 73 | sequence 2 shown in | |
| | FIG. 5E | |
| 74 | sequence 3 shown in | |
| | FIG. 5E | |
| 75 | sequence 4 shown in | |
| | FIG. 5E | |
| 76 | second sequence | fibrinogen α-chain isoform αE, amino acids 633-866 |
| | shown in FIG. 7 | |
| 77 | MMP target sites in | amino acid sequence with MMP target sites in human |
| | α-chain wt | fibrinogen α-chain unmodified |
| 78 | MMP target sites in | amino acid sequence with MMP target sites in human |
| | α-chain substituted | fibrinogen α-chain with modified amino acids |
| 79 | His-rFbgH433R435 | human fibrinogen α-chain isoform alpha with |
| | α-chain | substitutions L233H and T235R |
| 80 | His-rFbgH433R435 | human fibrinogen γ-chain isoform gamma with |
| | γ-chain | substitutions K88H, K111H, K382H and K399H |
| 81 | sequence shown in | |
| | FIG. 20A | |
| 82 | sequence shown in | |
| | FIG. 20B | |

The isolated recombinant fibrinogen of the present invention is characterized in that
(a) in the fibrinogen alpha-chains, with reference to the respective positions in SEQ ID NO: 2 (or SEQ ID NO:1 for all residues except for R687 and R847, since they are not found in SEQ ID NO: 1), one or more lysine (K) and/or arginine (R) residues selected from the group consisting R38, R42, K97, K100, R114, R123, R129, R135, K210, R216, R218, K225, K227, K238, K243, K249, R258, R271, R287, R308, R353, R367, R425, R426, K432, K437, K440, R443, K446, K448, R458, R459, K463, K467, K476, K480, R510, R512, K527, R547, K558, R573, K575, K581, R591, K599, K602, K620, R621, K625, R687 and R847 are substituted or deleted; and/or
(b) in the fibrinogen beta-chain, with reference to the respective positions in SEQ ID NO: 3, one or more lysine (K) and/or arginine (R) residues selected from the group consisting of R44, K51, K52, R53, R60, R72, K77, K83, R87, K152, R158, K160, K163, K178, R285, K328, R334, K348, K353, K367, K374, K458 and K471 are substituted or deleted; and/or
(c) in the fibrinogen gamma-chains, with reference to the respective positions in SEQ ID NO: 4, one or more lysine (K) and/or arginine (R) residues selected from the group consisting of R40, K79, K84, K88, K111, K114, K185, K199, K238, K292, R301, K382 and K399 are substituted or deleted.

In the context of the present invention the term “with reference to the respective positions in SEQ ID NO . . . ” is to be understood to only mean that the thus referred to amino acid sequence acts as a reference sequence for the respective indicated K or R residue in the respective fibrinogen chain. In other words, SEQ ID NO:2 (or equivalently SEQ ID NO:1 for all residues of the alpha-chains except those residues that they are not found in SEQ ID NO: 1, i.e. R687 and R847), 3 and 4 in the context of this term act merely as a reference system to clearly identify the K and R residues without limiting the respective sequence of the alpha-, beta- and gamma-chains of the inventive isolated recombinant fibrinogen in any way that goes beyond this identification of the K and R residue positions.

It does, for example, not mean that the respective alpha-, beta- and/or gamma chain(s) of the inventive isolated recombinant fibrinogen, defined in such a manner, comprise(s) all of the thus referenced sequences, i.e. all of SEQ ID NOs: 1, 3 and 4. On the contrary in many of the preferred embodiments of the invention, the alpha-, beta- and/or gamma chain of the inventive isolated recombinant fibrinogen comprise only a fragment of the amino acid sequences indicated in SEQ ID NOs: 1, 3 and 4 or a variant of such a fragment. Such fragments of the full-length wild-type sequences are for example shown in SEQ ID NOs: 6 to 11. Furthermore, the amino acid sequence of the alpha-, beta- and/or gamma chain(s) of the inventive isolated recombinant fibrinogen may comprise one or multiple sequence variations (apart from those K and R residues listed above) even compared to one of those alpha-, beta- and/or gamma chain fragments of e.g. SEQ ID NOs: 6 to 11.

The isolated recombinant fibrinogen of the present invention may comprise one or more substitutions or deletions in one or more of the K and R residues as indicated above, or as indicated in any of the more specific embodiments below, in the fibrinogen alpha-chains, and/or in the fibrinogen beta-chains, and/or in the fibrinogen gamma-chains. This means the isolated recombinant fibrinogen comprises one, or two, or three, or four, or five, or six, or seven, or eight, or nine, etc. substitutions or deletions of the above-indicated K and R residues in one or more chains of the recombinant fibrinogen, e.g. in one or preferably two gamma-chains, and/or one or preferably two beta-chains, and/or one or preferably two alpha-chains.

For example, in one embodiment of the present invention, the isolated recombinant fibrinogen comprises substitutions or deletions in, for example, 1, or 2, or 3, or 4, or 5, or 6, or 7, or 8, or 9, or 10 K and/or R residues in the gamma-chain sequence as indicated above, in the gamma-chains of the isolated recombinant fibrinogen. In another embodiment, the isolated recombinant fibrinogen comprises substitutions or deletions in, for example, 1, or 2, or 3, or 4, or 5, or 6, or 7, or 8, or 9, or 10, . . . , or 18, or 19 K and/or R residues in the beta-chain sequence as indicated above, in the beta-chains of the isolated recombinant fibrinogen. In yet another embodiment, the isolated recombinant fibrinogen comprises substitutions or deletions in, for example, 1, or 2, or 3, or 4, or 5, or 6, or 7, or 8, or 9, or 10, . . . , or 46, or 47 K and/or R residues in the alpha-chain sequence as indicated above, in the alpha-chains of the isolated recombinant fibrinogen. In yet another embodiment the isolated recombinant fibrinogen of the present invention comprises a combination of the above indicated substitutions or deletions in the alpha-chains, beta-chains, and/or gamma-chains. In a preferred embodiment the two alpha-chains and/or the two beta-chains and/or the two gamma chains of the isolated recombinant fibrinogen have the same substitutions or deletions, preferably substitutions, in the one or more of the K and R residues.

In a preferred embodiment, the isolated recombinant fibrinogen comprises substitutions or deletions, preferably substitutions, in at least 2, preferably at least 3, more preferably at least 4, e.g. in 5, or in 6, K and/or R residues in the gamma-chain sequence as indicated above, in one, or preferably both, gamma-chains of the isolated recombinant fibrinogen, and optionally no substitutions in the K and/or R residues identified above of the alpha- and beta-chains. An equivalent embodiment with the alpha-chains, and an equivalent embodiment with the beta-chains, are alternatively preferred embodiments of the invention.

According to a specific embodiment of the present invention, one or more substitutions or deletions in the alpha-chain and/or beta-chain and/or gamma-chain are present in both alpha-chains and/or both beta-chains and/or both gamma-chains of the isolated recombinant fibrinogen, such that there is no difference between the two alpha-chains and/or the two beta-chains and/or the two gamma-chains with respect to the one or more substitutions or deletions.

According to one embodiment, the isolated recombinant fibrinogen is characterized in that in one, or both, fibrinogen alpha-chains, with reference to the respective positions in SEQ ID NO: 1 (or SEQ ID NO: 2), one or more lysine (K) and/or arginine (R) residues selected from the group consisting R38, R42, K97, K100, R114, R123, R129, R135, K210, R216, K225, K238, K243, K249, R258, R271, R287, R308, R425, R426, K432, K437, K440, R443, K446, K448, R458, R459, K463, K467, K476, K480, R510, R512, K527, R547, K558, R573, K575, R591, K599, K602, K620, R621 and K625 are substituted or deleted, preferably substituted; and/or in one, or both, fibrinogen beta-chains, with reference to the respective positions in SEQ ID NO: 3, one or more lysine (K) and/or arginine (R) residues selected from the group consisting of R44, K51, K52, R53, R72, K77, K83, K152, R158, K160, K163, K178, R334, K348, K353, K367, K374, K458 and K471 are substituted or deleted, preferably substituted; and/or in one, or both, fibrinogen gamma-chains, with reference to the respective positions in SEQ ID NO: 4, one or more lysine (K) and/or arginine (R) residues selected from the group consisting of K79, K84, K88, K111, K114, K185, K292, R301, K382 and K399 are substituted or deleted, preferably substituted.

According to a preferred embodiment of the present invention, the one or more substituted or deleted K and R residues in the fibrinogen alpha-chains, with reference to the respective positions in SEQ ID NO: 1, comprise, or consist of, one or more of R135, K210, K243, R287, R308, R425, R426, K432, K437, K440, K446, K448, R458, R459, K463, K467, K480, R512, R547, K558, R573, K575, R591, K599, K620, R621, K625 and combinations thereof.

Additionally, according to a preferred embodiment of the present invention, the one or more substituted or deleted K and R residues in the fibrinogen beta-chains, with reference to the respective positions in SEQ ID NO: 3, comprise, or consist of, one or more of R44, K51, R53, K77, R158, K160, K178, R334, K348, K353, K367, K374, K458, K471, and combinations thereof. Additionally, according to a preferred embodiment of the present invention, the one or more substituted or deleted K and R residues in the fibrinogen gamma-chains, with reference to the respective positions in SEQ ID NO: 4, comprise, or consist of, K292, R301, or both. In another preferred embodiment the present invention, in addition the one or more substituted or deleted K and R residues indicated in this paragraph, the isolated recombinant fibrinogen of the present invention comprises one or more K and R residues indicated in the paragraph below.

According to another preferred embodiment of the present invention, the one or more substituted or deleted K and R residues in the fibrinogen alpha-chains, with reference to the respective positions in SEQ ID NO: 1 (or SEQ ID NO: 2), comprise, or consist of, one or more of R38, R42, K97, K100, R114, R123, R129, R135, R216, K225, K238, K249, R258, R271, R443, R510, K527 and K602; and/or the one or more substituted or deleted K and R residues in the fibrinogen beta-chains, with reference to the respective positions in SEQ ID NO: 3, comprise, or consist of, one or more of K52, R72, K83, K152 and K163; and/or the one or more substituted or deleted K and R residues in the fibrinogen gamma-chains, with reference to the respective positions in SEQ ID NO: 4, comprise, or consist of, one or more of K79, K84, K88, K111, K114, K185, K382 and K399, preferably K88, K111, K382 and K399. According to yet another preferred embodiment, the one or more substituted or deleted K and R residues in the fibrinogen alpha-chains, with reference to the respective positions in SEQ ID NO: 1 (or SEQ ID NO: 2), comprise, or consist of K100, R114, R123, R129, R135, K210, R216, K225, K238, K243, K249, R258, R271, R287, R308, R425, R426, K432, K437, K440, R443, K446, R458, R459, K463, K467, K476, K480, R510, R512, K527, R547, K558, R573, K575, R591, K599, K602, K620, K621 and combinations thereof.

According to yet another preferred embodiment of the present invention, the one or more substituted or deleted K and R residues in the fibrinogen alpha-chains, with reference to the respective positions in SEQ ID NO: 1 (or SEQ ID NO: 2), comprise, or consist of K97, K100, R114, R123, R129, R135, K210, R216, K225, K238, K243, K249, R258, R271, R287, R308, R425, R426, K432, K437, K440, R443, K446, R458, R459, K463, K467, K476, K480, R510, R512, K527, R547, K558, R573, K575, R591, K599, K602, K620, K621, K625 and combinations thereof, preferably K97, K100, R123, K225, K238, K243, R510, R512, K527, K599, K602, K620, R621, K625 and combinations thereof, more preferably K100, R123, K225, K238, R510, K527, K602, K620 and combinations thereof.

According to yet another preferred embodiment of the present invention, the one or more substituted or deleted K and R residues in the fibrinogen beta-chains, with reference to the respective positions in SEQ ID NO: 3, comprise, or consist of, R44, K51, K52, R53, R72, K77, K83, K152, R158, K160, K163, K178, R334, K348, K353, K367, K374, K458, K471 and combinations thereof, preferably K51, K52, R53, R72, K77, K152, R158, K160, K163, and combinations thereof, more preferably K52, R72, K152, K160 and combinations thereof.

According to yet another preferred embodiment of the present invention, the one or more substituted or deleted K and R residues in the fibrinogen gamma-chains, with reference to the respective positions in SEQ ID NO: 4, comprise, or consist of, one or more of K79, K84, K88, K111, K114, K382 and K399 and combinations thereof, preferably K79, K84, K88, K111, K382 and K399 and combinations, more preferably K79, K88, K111, K382, K399 and combinations thereof, or K88, K111, K382, K399 and combinations thereof.

According to a further preferred embodiment of the present invention, the one or more substituted or deleted K and R residues in the fibrinogen alpha-chains, with reference to the respective positions in SEQ ID NO: 1 (or SEQ ID NO: 2), comprise, or consist of K100, R114, R123, K225, K238, K249, R258, R271, R287, R308, R425, R426, K432, R443, R458, K467, R510, K527, R547, K558, K575, K599, K602, K621 and combinations thereof; and/or the one or more substituted or deleted K and R residues in the fibrinogen beta-chains, with reference to the respective positions in SEQ ID NO: 3, comprise, or consist of, R44, K51, K52, R72, K83, K152, R158, K160, K163 and combinations thereof; and/or the one or more substituted or deleted K and R residues in the fibrinogen gamma-chains, with reference to the respective positions in SEQ ID NO: 4, comprise, or consist of, one or more of K79, K88, K111, K382, K399 and combinations thereof.

In a specific embodiment, the isolated recombinant fibrinogen of the present invention, with reference to the respective positions in SEQ ID NO: 4, comprise two, or three, or four, or all, of the K and R residues K79, K88, K111, K382 and K399, in one or both fibrinogen gamma-chains. According to another specific embodiment all K and R residues indicated in any of the embodiments above, are deleted or substituted, preferably substituted. In some embodiments, the fibrinogen alpha-, beta-, and gamma-chains, apart from the K and R residues specified in any of the embodiments above, comprise no further mutations in K and R residues.

The one or more K and R residues as specified above may be substituted or deleted. How to carry out such amino acid substitutions and deletions is known in the art. According to a preferred embodiment the one or more K and R residues as specified above are substituted. For example, the fibrinogen alpha-, beta-, and gamma-chains of the isolated recombinant fibrinogen of the present invention may comprise only substitutions, and no deletions, of K or R residues.

The amino acid or amino acids used to substitute the K and R residues indicated above are not particularly limited. According to a preferred embodiment the one or more K or R residues are substituted with amino acids or isotopes of these amino acids selected from the group consisting of alanine (A), histidine (H), N6-[(2-azidoethoxy)carbonyl]-L-lysine, propargyl-L-lysine, azidohomoalanine, trans-cyclooct-2-en-L-lysine, exo BCN-L-lysine, trans-cyclooct-4-en-L-lysine, pyrrolysine and pyrrolysine analogs (e. g. N^{ε}-cyclopentyloxycarbonyl-L-lysine), lysine-nitrobenzyl-oxycarbonyl-N^{ε}-L-lysine, O-methyl-L-tyrosin and analogs, methionine (M), isoleucine (1), leucine (L), phenylalanine (F), tryptophane (W), preferably selected from alanine (A), histidine (H), N6-[(2-azidoethoxy)carbonyl]-L-lysine, propargyl-L-lysine, azidohomo-alanine, methionine (M), isoleucine (1), leucine (L); more preferably selected from alanine (A) or histidine (H). A particularly preferred amino acid is histidine (H). According to a specific embodiment, those of the one or more K or R residues, which are located in an α-helical region of an alpha-, beta-, and/or gamma-chain, are substituted with amino acids or isotopes of these amino acids selected from the group consisting of alanine (A), histidine (H), N6-[(2-azidoethoxy)carbonyl]-L-lysine, propargyl-L-lysine, azidohomoalanine, trans-cyclooct-2-en-L-lysine, exo BCN-L-lysine, trans-cyclooct-4-en-L-lysine, pyrrolysine and pyrrolysine analogs (e. g. N^{ε}-cyclopentyloxycarbonyl-L-lysine), lysine-nitrobenzyl-oxycarbonyl-N^{ε}-L-lysine, O-methyl-L-tyrosin and analogs, methionine (M), isoleucine (1), leucine (L); preferably alanine (A), histidine (H), N6-[(2-azidoethoxy)carbonyl]-L-lysine, propargyl-L-lysine, or azidohomoalanine; more preferably alanine (A) or histidine (H); most preferably with histidine (H).

According to another preferred embodiment of the present invention, the one or more substituted or deleted K and R residues in the fibrinogen gamma-chains, with reference to the respective positions in SEQ ID NO: 4, comprise, or consist of, K88, K111, K382 and K399, wherein these residues are substituted with histidine (H). According to yet another preferred embodiment of the present invention, the one or more substituted or deleted K and R residues in the fibrinogen gamma-chains, with reference to the respective positions in SEQ ID NO: 4, comprise, or consist of, K88, K111, K382 and K399, wherein these residues are substituted with alanine (A).

In addition to the one or more K and R residues, other amino acids in the direct vicinity of said one or more K and R residues may be mutated to further strengthen the effect against plasmin digestion. According to one embodiment, in addition to the one or more substituted or deleted K and R residues in the fibrinogen alpha-chain(s), and/or the one or more substituted or deleted K and R residues in the fibrinogen beta-chain(s), and/or the one or more substituted or deleted K and R residues in the fibrinogen gamma-chain(s), one or more, for example 1, 2, 3 or 4 amino acids, inside 3, preferably 2, amino acids directly up- and/or downstream in the reference sequence (i.e. in SEQ ID NO: 1, 3, and/or 4) of (some or all of) said one or more substituted or deleted K and R residues, are also substituted or deleted, preferably substituted.

According to another embodiment, in addition to the one or more substituted or deleted K and R residues in the fibrinogen alpha-chain(s), and/or the one or more substituted or deleted K and R residues in the fibrinogen beta-chain(s), and/or the one or more substituted or deleted K and R residues in the fibrinogen gamma-chain(s), all K residues, or all R residues, or all K and R residues, inside 2, preferably 3, more preferably 4, even more preferably 5, even more preferably 7, or 10, or 12, or 15, or 20 amino acids directly up- and/or downstream, in the reference sequence (i.e. in SEQ ID NO: 1, 3, and/or 4), of (some or all of) said one or more substituted or deleted K and R residues, are also substituted or deleted, preferably substituted.

According to yet another embodiment of the present invention, in addition to the one or more substituted or deleted K and R residues in the fibrinogen alpha-chain(s), and/or the one or more substituted or deleted K and R residues in the fibrinogen beta-chain(s), and/or the one or more substituted or deleted K and R residues in the fibrinogen gamma-chain(s), one or both amino acids directly adjacent in the reference sequence (i.e. in SEQ ID NO: 1, 3, and/or 4) to (some or all of) said one or more substituted or deleted K and R residues, are also substituted or deleted, preferably substituted.

According to yet another embodiment, none of the amino acids, apart from the one or more substituted or deleted K and R residues in the fibrinogen alpha-chain(s), and/or the one or more substituted or deleted K and R residues in the fibrinogen beta-chain(s), and/or the one or more substituted or deleted K and R residues in the fibrinogen gamma-chain(s), in the reference sequence (i.e. in SEQ ID NO: 1, 3, and/or 4), directly adjacent to, or within two amino acids of, (some or all of) said one or more substituted or deleted K and R residues, are substituted or deleted.

The amino acid sequences of the human fibrinogen derived alpha-, beta-, and gamma chains of the isolated recombinant fibrinogen of the present invention, apart from the substitutions or deletions one or more of K and R residues as set out above, are not particularly limited. The isolated recombinant fibrinogen of the present invention may comprise, for example, one isoform or two isoforms of alpha-chain and/or gamma-chain, i.e. either e.g. homodimers of alpha-chain and gamma-chain, Aα/Aα-Bβ/Bβ-γ/γ or AαE/AαE-Bβ/Bβ-γ/γ′, or Aα/Aα-Bβ/Bβ-γ/γ, etc., or heterodimers of alpha-chains and/or gamma-chains, Aα/AαE-Bβ/Bβ-γ/γ or Aα/AαE-Bβ/Bβ-γ′/γ or AαE/AαE-Bβ/Bβ-γ′/γ etc.

Moreover, each of the three subunits of the isolated recombinant fibrinogen may comprise one wild-type form or two wild-type forms, as long as at least one of the indicated lysine (K) and/or arginine (R) residues in at least one of the fibrinogen alpha-chains, or in at least one of the fibrinogen beta-chains, or in at least one of the fibrinogen gamma-chains is substituted or deleted. Additionally, the isolated recombinant fibrinogen of the present invention may comprise two different variants of fibrinogen alpha-chain, and/or of fibrinogen beta-chain, and/or of fibrinogen gamma-chains, for example two different variants of the same fibrinogen alpha-chain isoform, and/or the same fibrinogen gamma-chain isoform.

The alpha-, beta- and/or gamma-chain variants may, or may not, in sequence length correspond to the respective wild-type chain sequence in secreted form, or may be a truncated version of such chains, and may further comprise sequence variations, e.g. in the form of substitutions, deletions, and insertions relative to the wild-sequence, beyond those in the K and R residues of the present invention. In case of such truncated and/or otherwise mutated variants of any of the fibrinogen chains, preferably such variants still form intact fibrinogen, in the sense that fibrinogen can correctly fold and has apart from any intended post-translational modifications not been enzymatically cleaved, and preferably are functional, which preferably means that the isolated recombinant fibrinogen formed from such variants is biologically active and can, when brought in contact with the right initiator, polymerize to form a stable fibrin polymer (fibrin clot). Thus, as used herein, a “functional fibrinogen” preferably means a fibrinogen having an activity qualitatively the same as the physiological activity of a wild-type fibrinogen, while the quantitative factors such as the level of activity, molecular weight and the like may be different.

In the inventive isolated recombinant fibrinogen, according to a preferred embodiment
i. at least one, or preferably each, fibrinogen alpha-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, or 100%, identity with any of SEQ ID NOs: 6, 7, 8, 41, 42, 43, 45, 46, 47, 48, 49, and 50, preferably with SEQ ID NO: 6, or with SEQ ID NO: 7, or with SEQ ID NO: 8, or with SEQ ID NO: 41, or with SEQ ID NO: 42, or with SEQ ID NO: 43, more preferably with SEQ ID NO: 6, SEQ ID NO: 7, or SEQ ID NO: 8; and/or
ii. at least one, or preferably each, fibrinogen beta-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, or 100%, identity with SEQ ID NO: 9, or with SEQ ID NO: 44, preferably with SEQ ID NO: 9; and/or
iii. at least one, or preferably each, fibrinogen gamma-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, or 100%, identity with SEQ ID NO: 10, or with SEQ ID NO: 11.

The term “excluding the one or more substituted or deleted K and R residues” in the above context means that the sequence identity is calculated after exclusion of any K and R residue or residues which are substituted or deleted in accordance with the teaching of the present invention (i.e. excluding those K and R residue or residues substituted or deleted to achieve the effect of increased plasmin resistance as taught herein).

According to another preferred embodiment, in the isolated recombinant fibrinogen, i. each fibrinogen alpha-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, or 100%, identity with SEQ ID NO: 6 or SEQ ID NO: 7, preferably with SEQ ID NO: 7; and ii. each fibrinogen beta-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, or 100%, identity with SEQ ID NO: 9; and iii. each fibrinogen gamma-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, or 100%, identity with SEQ ID NO: 10.

According to yet another preferred embodiment, in the isolated recombinant fibrinogen, i. each fibrinogen alpha-chain comprises, or consists of, an amino acid sequence, which, preferably excluding the one or more substituted or deleted K and R residues (i.e. those K and R residues substituted or deleted to achieve the effect of increased plasmin resistance as taught herein), has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, or 100%, identity with SEQ ID NO: 8; and ii. each fibrinogen beta-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, or 100%, identity with SEQ ID NO: 9, or with SEQ ID NO: 44, preferably with SEQ ID NO: 9; and iii. each fibrinogen gamma-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, or 99.6%, or 99.7%, or 100%, identity with SEQ ID NO: 11.

According to another preferred embodiment, in the isolated recombinant fibrinogen of the present invention, one, or preferably each, fibrinogen alpha-chain comprises, or consists of, an amino acid sequence having, apart from the one or more substituted or deleted K and R residues (i.e. those K and R residues substituted or deleted to achieve the effect of increased plasmin resistance as taught herein), the sequence of any of SEQ ID NOs: 6, 7, 8, 41, 42, 43, 45, 46, 47, 48, 49, and 50, preferably of SEQ ID NO: 6, of SEQ ID NO: 7, or of SEQ ID NO: 8, or of SEQ ID NO: 41, or of SEQ ID NO: 42, or of SEQ ID NO: 43, more preferably of SEQ ID NO: 6, SEQ ID NO: 7, or SEQ ID NO: 8; and/or one, or preferably each, fibrinogen beta-chain comprises, or consists of, an amino acid sequence having, apart from the one or more substituted or deleted K and R residues, the sequence of SEQ ID NO: 9, or of SEQ ID NO: 44, preferably of SEQ ID NO: 9; and/or one, or preferably each, fibrinogen gamma-chain comprises, or consists of, an amino acid sequence having, apart from the one or more substituted or deleted K and R residues, the sequence of SEQ ID NO: 10, or of SEQ ID NO: 11.

Furthermore, the isolated recombinant fibrinogen may comprise post-translational modifications. Examples of such post-translational modifications are phosphorylation, glycosylation, hydroxylation, pyrrolidone carboxylic acid formation, lipidation, methylation, acetylation, oxidation or sulfation.

Preferably the isolated recombinant fibrinogen of the present invention is soluble. Moreover, isolated recombinant fibrinogen is preferably functional, wherein functional for example means that the recombinant fibrinogen is biologically active and that the polymerisation behaviour of the recombinant fibrinogen is not qualitatively different, and at least not significantly altered (although it may be different), compared to human wild-type fibrinogen. Thus, as used herein, a “functional fibrinogen” may preferably refer to a fibrinogen having an activity qualitatively the same as the physiological activity of a wild-type fibrinogen, while the quantitative factors such as the level of activity, molecular weight and the like may be different.

In a second aspect the present invention is directed to a novel fibrin sealant comprising isolated recombinant fibrinogen of the first aspect of the present invention as detailed in any of the embodiments described above, and optionally thrombin. The term “fibrin sealant” or “fibrin glue” is known in the art and preferably refers to a biological adhesive comprising fibrinogen or a fibrinogen derivative, whose effect imitates the final stages of coagulation to form a fibrin polymer. This makes fibrin sealants for example useful in surgery to control bleeding or to adhere two tissues to each other. Fibrin sealants and methods and processes to prepare such fibrin sealants are known in the art.

A very common type of fibrin sealant uses fibrinogen and comprises two components. One component comprises concentrated human fibrinogen, (e.g. bovine) aprotinin and Factor XIII (see e.g. U.S. Pat. No. 8,821,861 B2, US 2007/0231372 A1 and US 2014/0205636 A1). The second component comprises (e.g. bovine or human) thrombin and calcium chloride. Application of this type of sealant can be carried out for example with a double-barrelled syringe, which permits simultaneous delivery of both components to the desired site of the fibrin clot formation. The mixing of the two components at the target site produces a fibrin clot via a sequence of reactions.

A second type of fibrin sealant uses compositions consisting primarily of fibrin I and/or fibrin II monomers (see e.g. U.S. Pat. No. 6,083,902 A and EP 0592242 A1). In these types of sealants, fibrin I monomers and/or fibrin II monomers and/or desBB fibrin monomers are prepared in advance of sealant application from fibrinogen using an appropriate proteolytic enzyme, such as thrombin. The fibrin monomers are maintained in soluble form using an appropriate buffer. The fibrin I monomers, fibrin II monomers or desBB fibrin monomers in such solutions can be converted to fibrin polymers by mixing the solution with a second solution to produce a mixture with conditions that permit the spontaneous polymerization of the fibrin monomers to form a fibrin clot.

Preferably the fibrin sealant further comprises thrombin, preferably human or bovine thrombin, more preferably human thrombin, as a second component to initiate fibrin polymerization, once the recombinant fibrinogen is contacted with the thrombin. Methods and processes to prepare thrombin are known in the art. As an alternative to thrombin, the fibrin sealant may comprise one or more other components, which when in contact with fibrinogen initiate and drive fibrin polymerization.

The fibrin sealant may comprise further components like for example a protransglutaminase, calcium chloride, polyphosphate (PolyP), Zn²⁺, fibronectin, and/or hydroxyapatite. Preferably the protransglutaminase is factor XIII (FXIII). Thus, according to a preferred embodiment, the inventive fibrin sealant comprises, preferably human or bovine, more preferably human, thrombin; and optionally one or more additives selected from the group consisting of protransglutaminase, preferably factor XIII (FXIII), one or more calcium salts (preferably calcium chloride), polyphosphate (PolyP), Zn²⁺, fibronectin, hydroxyapatite, growth factors (e. g. VEGF), one or more mono- and/or polysaccharides as well as sugar and poly alcohols (preferably selected from mannitol, sorbitol, trehalose) and cells (genetically modified or not; e. g. stem cells, genetically modified or not). According to a specific embodiment, the fibrin sealant comprises genetically modified (GMO) cells, preferably genetically modified stem cells. In yet another embodiment the fibrin sealant further comprises one or more active ingredients (e. g. antibiotics, cytokines, antibodies or growth factors).

The new inventive fibrin sealant comprising the isolated recombinant fibrinogen of the present invention has the advantage that addition of inhibitors of plasmin protease, and in particular aprotinin, is no longer required in order to ensure sustained stability of the fibrin clot once formed. Therefore, in a preferred embodiment the inventive fibrin sealant does not comprise aprotinin. According to another preferred embodiment the fibrin sealant of the present invention does not comprise any plasmin inhibitors. According to yet another embodiment the fibrin sealant of the present invention does not comprise any protease inhibitors.

In a third aspect the present invention is directed to a fibrin sealant kit comprising:
i) a container comprising isolated recombinant fibrinogen of the first aspect of the present invention as detailed in any of the embodiments described above; and
ii) a container comprising, preferably human- or bovine-derived, more preferably human, thrombin.

The fibrin sealant kit may comprise further components like for example a protransglutaminase, calcium chloride, polyphosphate (PolyP), Zn²⁺, fibronectin, and hydroxyapatite. Preferably the protransglutaminase is factor XIII (FXIII). Thus, according to a preferred embodiment, the inventive fibrin sealant kit comprises one or more additives selected from the group consisting of protransglutaminase, preferably factor XIII (FXIII), one or more calcium salts (preferably calcium chloride), polyphosphate (PolyP), Zn²⁺, fibronectin, hydroxyapatite, growth factors (e. g. VEGF), one or more mono-, di-, polysaccharides as well as sugar and poly alcohols (preferably selected from mannitol, sorbitol, trehalose) and cells (genetically modified or not; e. g. stem cells, genetically modified or not). According to a specific embodiment, the fibrin sealant kit comprises genetically modified (GMO) cells, preferably genetically modified stem cells. In yet another embodiment the fibrin sealant kit further comprises one or more active ingredients, preferably selected from the group consisting of active pharmaceutical ingredients (e. g. antibiotics, cytokines, antibodies or growth factors).

According to a preferred embodiment, Factor XIII is included in the container of the fibrin sealant kit comprising the recombinant fibrinogen. According to another preferred embodiment, the container of the fibrin sealant kit comprising the thrombin further comprises calcium chloride.

The new inventive fibrin sealant kit comprising the isolated recombinant fibrinogen of the present invention has the advantage that inhibitors of plasmin protease, and in particular aprotinin, is no longer required in order to ensure sustained stability of the fibrin clot once formed. Therefore, in a preferred embodiment the inventive fibrin sealant kit does not comprise aprotinin. According to another preferred embodiment the fibrin sealant kit of the present invention does not comprise any plasmin inhibitors. According to yet another embodiment the fibrin sealant kit of the present invention does not comprise any protease inhibitors.

The fibrin sealant and the fibrin sealant kit of the present invention can be used, for example, as a hemostat, a tissue sealant, or a wound adhesive. Therefore, according to one embodiment of the present invention, the fibrin sealant, or the fibrin sealant kit, is for use as a hemostat, a tissue sealant, or a wound adhesive. Furthermore, the fibrin sealant and the fibrin sealant kit are useful in soft tissue procedures and internal wound procedures. Therefore, according to another embodiment, the fibrin sealant, or the fibrin sealant kit of the present invention is for use in soft tissue procedures and/or internal wound procedures.

The fibrin sealant and the fibrin sealant kit of the present invention can be used, for example, for sealing a defect site or an incised surface of organs and tissues, or for sealing junction between incised tissues or between incised tissues and prosthetic materials. Thus, according to a specific embodiment, the inventive fibrin sealant, and the inventive fibrin sealant kit, are for use in sealing a defect site or an incised surface of organs and tissues, or for sealing junction between incised tissues or between incised tissues and prosthetic materials According to a fourth aspect, the present invention is directed to a eucaryotic cell, comprising exogenous nucleotide sequences encoding one or more recombinant fibrinogen alpha-chains, one or more recombinant fibrinogen beta-chains, and one or more recombinant fibrinogen gamma-chains; characterized in that
(a) in the fibrinogen alpha-chains, with reference to the respective positions in SEQ ID NO: 2, one or more lysine (K) and/or arginine (R) residues selected from the group consisting R38, R42, K97, K100, R114, R123, R129, R135, K210, R216, K225, K238, K243, K249, R258, R271, R287, R308, R425, R426, K432, K437, K440, R443, K446, K448, R458, R459, K463, K467, K476, K480, R510, R512, K527, R547, K558, R573, K575, R591, K599, K602, K620, R621, K625, R687 and R847 are substituted or deleted; and/or
(b) in the fibrinogen beta-chains, with reference to the respective positions in SEQ ID NO: 3, one or more lysine (K) and/or arginine (R) residues selected from the group consisting of R44, K51, K52, R53, R60, R72, K77, K83, R87, K152, R158, K160, K163, K178, R285, K328, R334, K348, K353, K367, K374, K458 and K471 are substituted or deleted; and/or (c) in the fibrinogen gamma-chains, with reference to the respective positions in SEQ ID NO: 4, one or more lysine (K) and/or arginine (R) residues selected from the group consisting of R40, K79, K84, K88, K111, K114, K185, K199, K238, K292, R301, K382 and K399 are substituted or deleted.

The term “exogenous” in this context means that at least part of one or more nucleotide sequences encoding one or more recombinant fibrinogen alpha-chains, one or more recombinant fibrinogen beta-chains, recombinant fibrinogen gamma-chains and/or one or more promoter sequences driving their expression is not originally part of the eucaryotic cell and is introduced by methods of molecular engineering. According to preferred embodiment, the eucaryotic cell comprises exogenous nucleotide sequences encoding no more than two recombinant fibrinogen alpha-chains, recombinant fibrinogen beta-chains, and recombinant fibrinogen gamma-chains. According to another preferred embodiment, the eucaryotic cell comprises exogenous nucleotide sequences encoding exactly one recombinant fibrinogen alpha-chain, and/or exactly one recombinant fibrinogen beta-chain, and/or exactly one recombinant fibrinogen gamma-chain.

Regarding the one or more substituted or deleted lysine (K) and/or arginine (R) residues in the fibrinogen alpha-, beta-, and/or gamma-chains, the embodiments relating to the inventive isolated recombinant fibrinogen of the first aspect as described above apply mutatis mutandis. Similarly, the sequence of the one or more fibrinogen alpha-chains, the one or more recombinant fibrinogen beta-chains, and the one or more recombinant fibrinogen gamma-chains of the isolated recombinant fibrinogen are as specified in the embodiments relating to the inventive isolated recombinant fibrinogen of the first aspect described above.

The eucaryotic cell is not particularly limited as long as it allows for genetic engineering resulting in a eucaryotic cell comprising exogenous nucleotide sequences encoding one or more recombinant fibrinogen alpha-chains, one/or more recombinant fibrinogen beta-chains, and/or one or more recombinant fibrinogen gamma-chains in accordance with the present invention. According to a preferred embodiment, the eucaryotic cell is a mammalian cell culture cell. Eucaryotic cells, and in particular mammalian cell culture cells, and methods for preparing such cells expressing a recombinant protein, and also specifically recombinant fibrinogen, are known in the art, see e.g. U.S. Pat. Nos. 10,208,101 and 6,037,457, and U.S. patent applications US 2010/0151522 A1, US 2010/0159512 A1 and US 2017/0037108 A1.

The eucaryotic cell of the present invention, when cultured under conditions wherein the fibrinogen is produced, preferably produces recombinant fibrinogen. The thus produced fibrinogen, preferably, is intact comprising undigested and correctly folded polypeptides, and, preferably, functional, such that it can be used in a fibrin sealant.

According to a preferred embodiment, the eucaryotic cell is a mammalian cell culture cell, preferably a Chinese hamster ovary (CHO) cell, or a human embryonic kidney (HEK) cell, more preferably a Chinese hamster ovary (CHO) cell. According to a preferred embodiment, the eucaryotic cell is a human embryonic kidney (HEK) cell, preferably selected from a HEK 293, a HEK 293T, a HEK 293S and a HEK 293 EBNA cell. According to another preferred embodiment, the eucaryotic cell is a murine myeloma cell, preferably selected from a NS0 cell, a NS-1 cell and a Sp2/0 cell. According to yet another preferred embodiment, the eucaryotic cell is a Baby hamster kidney (BHK) cell, preferably a BHK-21 cell. According to yet another preferred embodiment, the eucaryotic cell is a rat myeloma cell, preferably a YB2/0 cell or a YB2/3HL cell. According to a particularly preferred embodiment, the eucaryotic cells is a CHO cell, preferably selected from a CHO-K1, a CHO-DG44, a CHO-Pro minus and a CHO-S cell, and a PER.C6 cell, more preferably a CHO-S cell. A commercially available example of a suitable CHO-S cell is the ExpiCHO-S cell line from Thermo Fisher.

Exogenous nucleotide sequences encoding alpha-chain, beta-chain and gamma chain variants of fibrinogen can be introduced into a eucaryotic cell, for example, by using an expression vector. An expression vector using eucaryotic, preferably mammalian cell culture, cells as a host is not particularly limited, and an expression vector known in the art such as a plasmid vector, virus vector and the like can be appropriately selected. A promoter to be contained in such a fibrinogen expression vector is not particularly limited as long as it efficiently functions in a eucaryotic host cell to be used, and finally results in fibrinogen, preferably in a functional form. Examples include SV40 promoter, cytomegalovirus (CMV) promoter, RSV promoter, p actin promoter and the like. It is also possible to combine a promoter with a suitable enhancer. Such a fibrinogen expression vector may optionally comprise one or more selective marker genes, which are known in the art and not particularly limited. Other constituent elements (e.g., terminator and the like) optionally contained in a fibrinogen expression vector are not particularly limited.

Other ways of introducing exogenous nucleotide sequences encoding alpha-chain, beta-chain and gamma chain variants of fibrinogen into a eucaryotic host cell, or of introducing exogenous nucleotide sequences to modify alpha-chain, beta-chain and gamma-chain sequences already present in a eucaryotic host cell (and e.g. giving rise to expression of human derived fibrinogen already before introduction exogenous nucleotide sequences), using known methods of molecular engineering are conceivable. According to another preferred embodiment, at least one, and preferably each, of the exogenous nucleotide sequences is optimized for expression in the mammalian cell culture cells under conditions wherein the fibrinogen is produced.

In one embodiment, one fibrinogen expression vector is used for introducing the exogenous nucleotide sequence such that, for example, a single expression vector comprises all of genes encoding alpha-, beta- and gamma chains of fibrinogen. In another embodiment, the fibrinogen expression vector is composed of an expression vector having two of the genes encoding the alpha-, beta- and gamma chains of fibrinogen (e.g., alpha-chain and gamma-chain, beta-chain and gamma-chain and the like) and an expression vector having a remaining one. In yet another embodiment, the fibrinogen expression vector is composed of three expression vectors each containing genes encoding-alpha chain, beta-chain and gamma-chain of fibrinogen. When fibrinogen is expressed using two or more expression vectors, respective expression vectors may be simultaneously introduced into an eucaryotic cell, or sequentially introduced at different times by using, for example, a different selective marker, where the order of introduction is not particularly limited. In a preferred embodiment, the fibrinogen expression vector is a single expression vector containing, all genes encoding alpha-, beta- and gamma-chain of fibrinogen, for example at a constitution ratio of 1:1:1, preferably resulting in the expression at a constitution ratio of 1:1:1 in the eucaryotic cell.

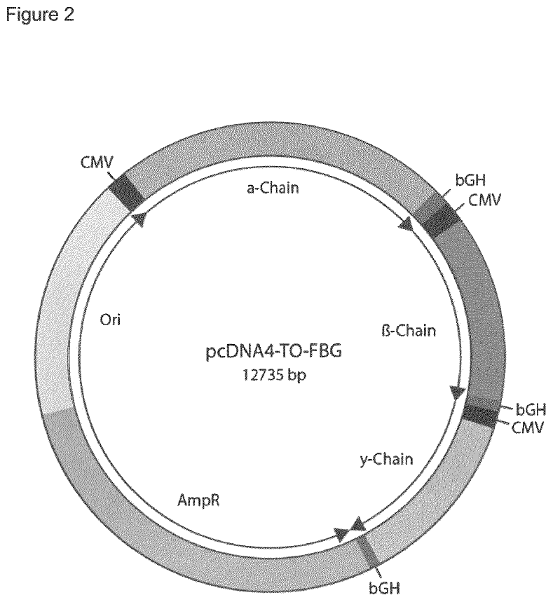

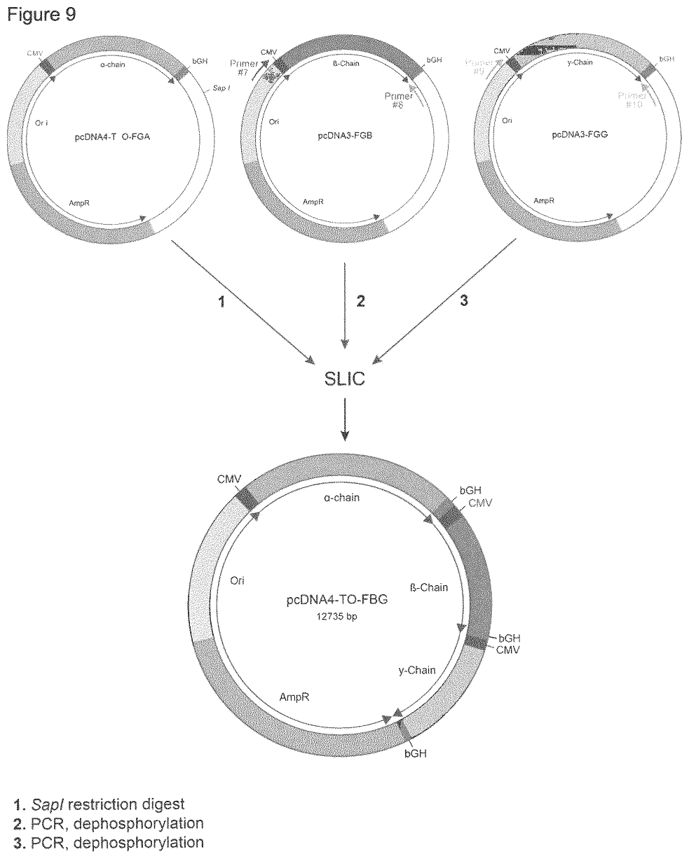

A preferable example of a single expression vector containing all genes of alpha-, beta- and gamma-chain of fibrinogen is one having three expression cassettes in which each gene encoding alpha-, beta- and gamma-chain of fibrinogen is under regulation of different promoters. The promoters regulating expression of each gene may be the same or different, and the same promoter (e.g., CMV promoter) is preferably used. See also FIGS. 2 and 9 and Table 2 for examples.

Alternatively, two or more of genes encoding alpha-, beta- and gamma-chains of fibrinogen may be under regulation of a single promoter. In this case, a sequence enabling polycistronic expression (e.g., IRES sequence, 2A sequence derived from foot-and-mouth disease virus and the like) can be inserted between each gene under control of a single promoter.

According to a fifth aspect the present invention is directed to a method for the production of isolated recombinant fibrinogen, preferably carrying one or more site-directed mutations relative to human wild-type fibrinogen, comprising the steps of
A) culturing eucaryotic cells comprising exogenous nucleotide sequences encoding at least one fibrinogen alpha-chain, at least one fibrinogen beta-chain, and at least one fibrinogen gamma-chain, preferably derived from mammalian, more preferably human, fibrinogen, in a culture medium under conditions wherein fibrinogen is produced; and
B) recovering the recombinant fibrinogen produced;
   wherein step A) is carried out in the presence of at least one matrix metalloproteinase (MMP) inhibitor, preferably selected from the group consisting of 1,10 Phenanthroline, UK 370106, GM6001, and a combination thereof, preferably UK 370106.

It is to be understood, that an MMP inhibitor in order to be suitable for this method of the fifth aspect has to be compatible for use with the eucaryotic cells during the culturing step A) (e.g. without, causing excessive cytotoxicity). Preferably the MMP inhibitor to be used in this method is selected from the group consisting of 1,10 Phenanthroline, UK 370106, GM6001, and a combination thereof. A particularly preferred MMP inhibitor is UK 370106.

UK 370106 with the formula (βR)-β-[[[(1 S)-1-[[[(1 S)-2-Methoxy-1-phenylethyl]amino]carbonyl]-2,2-dimethylpropyl]amino]carbonyl]-2-methyl-[1,1′-biphenyl]-4-hexanoic acid, is a highly selective MMP-3 and MMP-12 inhibitor. GM6001, also known as ilomastat or N-[(2R)-2-(hydroxamidocarbonylmethyl)-4-methylpentanoyl]-L-tryptophan methylamide, is a broad-spectrum matrix metalloprotease (MMP) inhibitor.

In step A) the MMP inhibitor(s) may be added directly to the culture medium, in step B) the MMP inhibitor(s) may be already present in the solution, recovered culture medium from step A), comprising the produced recombinant fibrinogen of step A). Step B) may be carried out in the presence of EDTA.

How to use 1,10 Phenanthroline, UK 370106, GM6001 and in particular suitable concentrations are known in the art. For UK 370106 a suitable concentration is for example at least 0.3 μM and no more than 150 μM, preferably at least 0.5 μM and no more than 100 μM, more preferably at least 1 μM and no more than 80 μM, even more preferably at least 5 μM and no more than 60 μM, even more preferably at least 10 μM and no more than 50 μM, even more preferably at least 20 μM and no more than 40 μM, e.g. about 25 μM, or about 30 μM, or about 35 μM, preferably about 25 μM. For 1,10 Phenanthroline a suitable concentration is for example at least 0.1 mM and no more than 10 mM, preferably at least 0.2 mM and no more than 5 mM, more preferably at least 0.5 mM and no more than 2 mM, e.g. about 0.6, or 0.8 or 1, or 1.2, or 1.4 mM, preferably about 1 mM. For GM6001 a suitable concentration is for example at least 250 nM and no more than 250 μM, preferably at least 2.5 μM and no more than 100 μM, more preferably at least 5 μM and no more than 50 μM, even more preferably 10 μM to 40 μM, or 10 μM to 25 μM, or 10 μM to 25 μM, e.g. about 10 or 15, or 20, or 25, or 30 μM, preferably about 25 μM.

According to a preferred embodiment, the inventive method in step B) comprises collecting at least a portion of the culture medium, preferably containing greater than 0.5 μg/ml, more preferably greater than 1 μg/ml, even more preferably greater than 2 μg/ml, even more preferably greater than 5 μg/ml, even more preferably greater than 10 μg/ml, even more preferably greater than 20 μg/ml, even more preferably greater than 50 μg/ml, even more preferably greater than 100 μg/ml, of recombinant fibrinogen.

According to another preferred embodiment, the inventive method in step B) comprises, optionally concentrating the fibrinogen from the culture medium to form a concentrated medium and, purifying the recombinant fibrinogen, preferably from the culture medium. According to a specific preferred embodiment, the purifying is carried out by chromatographic methods, preferably affinity chromatography.

The eucaryotic cells to be used in the inventive method are not particularly limited as long as they allow for the production of recombinant fibrinogen. According to a preferred embodiment, the eucaryotic cells are mammalian cell culture cells, preferably Chinese hamster ovary (CHO) cells, preferably selected from CHO-K1, CHO-DG44, CHO-Pro minus and CHO-S cells, PER.C6 cells, or human embryonic kidney (HEK) cells, preferably selected from HEK 293, HEK 293T, HEK 293S or HEK 293 EBNA cells, or murine myeloma cells, preferably selected from NS0 cells, NS-1 cells and Sp2/0 cells, or Baby hamster kidney (BHK) cells, preferably BHK-21 cells, or rat myeloma cells, preferably YB2/0 cells or YB2/3HL cells. Preferably, the eucaryotic cells are CHO cells, like CHO-S cells. A commercially available example of a suitable CHO-S cell is the ExpiCHO-S cell line from Thermo Fisher.

According to another preferred embodiment, at least one, and preferably all, of the exogenous nucleotide sequences is optimized for expression in the mammalian cell culture cells under conditions wherein the fibrinogen is produced. Regarding the eucaryotic cells and their preparation, the embodiments described under the fourth aspect of the present invention apply mutatis mutandis.

The method of this a fifth aspect is directed to the production of isolated recombinant fibrinogen, preferably carrying one or more site-directed mutations relative to the sequence of human wild-type fibrinogen. Thus, the isolated recombinant fibrinogen to be produced, according to a preferred embodiment is a mutant isolated recombinant fibrinogen that differs from the amino acid sequence of human wild-type fibrinogen or a fragment thereof (e.g. as defined in Table 1), in one or more amino acids, e.g. through substitution or deletion, preferably substitution. According to a specific embodiment of the method for the production of isolated recombinant fibrinogen, the isolated recombinant fibrinogen is the isolated recombinant fibrinogen according of any of the embodiments described above in the context of the first aspect of the present invention, and/or the eucaryotic cells are cells in accordance with any of the embodiments described above in the context of the fourth aspect of the present invention.

According to a sixth aspect, the present invention is directed to a method for identifying plasmin resistant recombinant fibrinogen variants comprising the steps of
A) identification of plasmin cleavage sites in fibrinogen, by
   i) incubating a fibrin gel, of preferably 10-30 mg/ml, more preferably 20 mg/ml density, with a plasmin solution of preferably 0.001 to 0.01 mU/ml activity;
   ii) collecting supernatant at different timepoints after addition of the plasmin solution, and optionally inactivating the plasmin, preferably by addition of PSMF and/or heat;
   iii) determining cleavage sites in fibrinogen, preferably using liquid chromatography mass spectrometry or solid phase extraction (SPE), more preferably using high performance liquid chromatography/electrospray ionization mass spectrometry (HPLC/ESI-MS), to obtain a time-resolved cleavage pattern of the fibrinogen; and
   iv) identifying lysine (K) and/or arginine (R) residues immediately before, and optionally immediately after, the cleavage sites in the amino acid sequence of fibrinogen; and
B) optionally selecting one or more of the identified lysine (K) and/or arginine (R) residues in the amino acid sequence of the fibrinogen, preferably based on the time-resolved cleavage pattern, for substitution or deletion, to obtain a new recombinant fibrinogen variant; and optionally incubating the new recombinant fibrinogen variant with plasmin to test for sensitive to proteolytic cleavage by plasmin.

In step A) i) a fibrin gel, of for example 10-30 mg/ml, preferably about 20 mg/ml density, is incubated with a plasmin solution, preferably by covering the fibrin gel with the plasmin solution. The plasmin solution preferably comprises plasmin equalling an activity of 0.0005 to 0.5 mU/ml, more preferably 0.0005 to 0.1 mU/ml, even more preferably 0.001 to 0.01 mU/ml activity; more preferably 0.002 to 0.02 mU/ml activity e.g. about 0.5 mU/ml. The plasmin is preferably human plasmin. The plasmin solution may comprise one or more other enzymes, for example one or more additional proteases.

How to prepare Fibrin gels is known in the art. The fibrin gel may be prepared by dialyzing fibrinogen against a suitable buffer (10 mM Tris/HCl, 150 mM NaCl, pH 7.5) at room temperature (RT). Gelation of fibrinogen with a concentration ranging from 0.5 to 106 mg/ml may be started by the addition of thrombin (1 U/ml), preferable also factor Xllla (50 mU/ml) and CaCl₂ (10 mM). Fibrinogen solutions may be incubated for at least two hours at RT.

In step A) ii) supernatant comprising the plasmin solution and digested fibrin is collected at different timepoints after addition of the plasmin solution. For example, the first time point for collection might be at t=0, e. g. just before (in which case the plasmin solution is used) or just after addition of the plasmin solution and can be used as reference sample. Further time points for collection may be equally spaced e.g. every 30 min, or every 20 min, or every 15 min, or every 10 min, etc. After collection the plasmin preferably inactivated (preferably immediately after sample collection), preferably by addition of a plasmin inhibitor, like phenylmethylsulfonyl fluoride (PMSF), and/or using heat. Heat inactivation may be carried out by incubation of the collected sample at 95° C. for e.g. 10 minutes. PSMF may be used at e.g. 0.5 to 2 mM, or at about 1 mM. Preferably the plasmin is inactivated using both PSMF and heat.

In step A) iii) cleavage sites in fibrinogen are determined to obtain a time-resolved cleavage pattern of fibrinogen. The method to do so is not particularly limited. Preferably liquid chromatography mass spectrometry or solid phase extraction (SPE) mass spectrometry is used. In specific preferred embodiment high performance liquid chromatography/electrospray ionization mass spectrometry (HPLC/ESI-MS) is used to determine the cleavage sites. The determination of cleavage sites might also involve quantification plasmin dependent cleavage of fibrinogen. For this quantification, according to a specific embodiment, an internal standard, for example angiotensin II, is used A) iii) for analysing the cleavage pattern.

In step A) iv) lysine (K) and/or arginine (R) residues immediately before and after the cleavage sites in the amino acid sequence of fibrinogen are identified, for example using bioinformatic tools known in the art.

Finally, in optional step B) one or more of the identified lysine (K) and/or arginine (R) residues in the amino acid sequence of fibrinogen are selected. The selection may for example be based the time-resolved cleavage pattern. The one or more of the identified lysine (K) and arginine (R) is substituted or deleted, preferably substituted, to obtain a new recombinant fibrinogen variant; and optionally the new recombinant fibrinogen variant (e.g. in solution and/or as part of a fibrin gel) is incubated with plasmin to test for sensitivity to proteolytic cleavage by plasmin.

According to one embodiment, the fibrin in the fibrin gel is based, preferably exclusively, on wild-type fibrinogen. According to another embodiment, the fibrin in the fibrin gel is based, preferably exclusively, on a mutant fibrinogen, preferably selected from one of the isolated recombinant fibrinogens according to any of the embodiments of the first aspect of the present invention as set out above.

### EXAMPLES

### Example 1

### Materials and Methods

### Materials

Restriction enzymes were purchased from New England Biolabs (NEB, Frankfurt a. M., Germany) and used according to manufacturer's instructions. For PCRs, Phusion™ High-Fidelity DNA Polymerase from ThermoFisherScientific (Waltham, USA) was used, also according to manufacturer's instructions. Custom oligonucleotides were purchased from SigmaAldrich/Merck (Darmstadt, Germany), desalted and lyophilized, and solubilized to 100 μM in H20 upon arrival. The ExpiCHO™ expression system, comprising Gibco ExpiCHO-S cells, Gibco ExpiCHO™ Expression Medium, Gibco ExpiFectamine™ CHO reagent, ExpiCHO™ Feed, ExpiFectamine™ Enhancer and Gibco OptiPRO™ Serum-Free Complexation Medium were purchased from ThermoFisherScientific (Waltham, USA). Spectra™ Prestained Multicolor High Range Protein Ladder (40-300 kDa), Spectra™ Prestained Multicolor Broad Range Protein Ladder (40-300 kDa) and PageRuler™ (10-170 kDa), Coomassie Brilliant Blue G250, SuperSignal™ West Pico PLUS Chemiluminescent Substrate, Bradford Protein Assay Kit, Mammalian Cell Lysis Buffer, 1-Step™ Ultra TMB ELISA Substrate Solution were from Thermo Fischer Scientific (Dreieich, Germany). HiTrap™ Capto MMC, Superdex™ 200 Hiload 16/600 and Superdex™ 75 10/300 GL were from Cytiva (Buckinghamshire, GB).

### Methods

### Site-Directed Mutagenesis

The γ-chain vector DNA was amplified using PCR with primers designed in order to insert mutations at different positions in the sequence (FGG-Lys88-S-FW: 5′-GCAGCTGATC AGCGCCATCC AGCTGACCTA CAACCCCGAC GAGG-3′; FGG-Lys88-S-RV: 5′-GGTCAGCTGG ATGGCGCTGA TCAGCTGCTT CACTTCGCTG GTC-3′; FGG-Lys111-S-FW: 5′-CCGCCACCCT GAGCTCCCGG AAGATGCTGG AAGAGATC-3′; FGG-Lys111-S-RV: 5′-CGGGAGCTCA GGGTGGCGGC GTCGATCATG TTG-3′; FGG-Lys356-S-FW: 5′-CTACAGCAGC GCCAGCACCC CCAACGGCTA CGACAAC-3′; FGG-Lys356-S-RV: 5′-GTGCTGGCGC TGCTGTAGGT GCCGCCCTGG TAGTACACG-3′; FGG-Lys373-S-FW: 5′-CACCTGGAGC ACCCGGTGGT ACAGCATGAA GAAAACCAC-3′; FGG-Lys373-S-RV: 5′-CCGGGTGCTC CAGGTGGCCC AGATGATGCC GTTGT-3′). The amplified cDNA was transformed into E. coli dH5α and mutations of target residues were verified by sanger sequencing.

TABLE 2
Listing of the different plasmids

| Date/Name | FGA | FGB | FGG | tag |
|---|---|---|---|---|
| pcDNA4/TO/FG | wt, | wt, | wt, | C-terminal |
| | isoform α-E | isoform 1 | isoform γ′ | myc-His (FGA) |
| pcDNA4/TO/FG_wt_no tag | wt, | wt, | wt, | — |
| | isoform α-E | isoform 1 | isoform γ′ | |
| pcDNA4/TO/FG_mut_mycHis | wt, | wt, | K88S, | C-terminal |
| | isoform α-E | isoform 1 | K111S, | myc-His (FGA) |
| | | | K382S, | |
| | | | K399S, | |
| | | | isoform γ′ | |
| pcDNA4/TO/FG_mut_no tag | wt, | wt | K88S, | — |
| | isoform α-E | isoform 1 | K111S, | |
| | | | K382S, | |
| | | | K399S, | |
| | | | isoform γ′ | |
| pcDNA4/TO/FG_new_wt | wt, | wt, | wt, | — |
| | isoform α | isoform 1 | isoform γ | |
| pcDNA4/TO/FG_new_HisM4 | wt, | wt, | K88H, | — |
| | isoform α | isoform 1 | K111H, | |
| | | | K382H, | |
| | | | K399H, | |
| | | | isoform γ | |
| pcDNA4/TO/FG_new_AlaM3 | wt, | wt | K88A, | — |
| | isoform α | isoform 1 | K111A, | |
| | | | K382A, | |
| | | | K399A, | |
| | | | isoform γ | |
| pcDNA4/TO/FG_K432H_wt | K432H, | wt, | wt, | — |
| | isoform α | isoform 1 | isoform γ | |
| pcDNA4/TO/FG_5 × KtoH_wt | 5 × KtoH*, | wt, | wt, | — |
| | isoform α | isoform 1 | isoform | |
| pcDNA4/TO/FG_K432H_HisM4 | K432H, | wt, | K88H, | — |
| | isoform α | isoform 1 | K111H, | |
| | | | K382H, | |
| | | | K399H, | |
| | | | isoform γ | |
| pcDNA4/TO/FG_5 × | 5 × KtoH*, | wt, | K88H, | — |
| KtoH_HisM4 | isoform α | isoform 1 | K111H, | |
| | | | K382H, | |
| | | | K399H, | |
| | | | isoform γ | |

TABLE 3
Oligonucleotide sequences

| Oligonucleotide | Direction | Sequence |
|---|---|---|
| #1 | forward | TGGCGGCCGCTCGAGTCTAGGCCACCATGGTGTCCA |
| | | TGC |
| #2 | reverse | TTGTTCGAAGGGCCCTCTAGCCTGAGTGACCAGGGG |
| | | C |
| #3 | forward | GCCGCCAGTGTGCTGGAATTGCCACCATGGTCCGGA |
| | | TGG |
| #4 | reverse | TGATGGATATCTGCAGAATTTCACTGCTGAGGAAAG |
| | | AATGGCC |
| #5 | forward | GCCGCCAGTGTGCTGGAATTGCCACCATGGTCTGGT |
| | | CCC |
| #6 | reverse | TGATGGATATCTGCAGAATTTCACAGGTCGTCCTCG |
| | | GGG |
| #7 | forward | GTATTGGGCGCTCTTCCGCTGTACGGGCCAGATATA |
| | | CGCG |
| #8 | reverse | CGCGTATATCTGGCCCGTACCCATAGAGCCCACCGC |
| | | ATCC |
| #9 | forward | GGATGCGGTGGGCTCTATGGGTACGGGCCAGATATA |
| | | CGCG |
| #10 | reverse | GAGTCAGTGAGCGAGGAAGCCCATAGAGCCCACCG |
| | | CATCC |
| #68 | reverse | TGTTCGAAGGGCCCTCTAGATCAAGCCTGAGTGACC |
| | | AGGGGCC |
| #198 | reverse | TTGTTCGAAGGGCCCTCTAGCTAGGGGGACAGGGA |
| | | AG |
| #201 | forward | TAAGAAGGAGATATACCATGGAAGCAGCAGTCG |
| #302 | forward | CATCTGGTCACTTCTAAAGGAGATAAAGAGC |
| #303 | forward | CATCTGGTCACTTCTCATGGAGATCATGAGCTCAGG |
| | | ACTGGTCATGAGCATGTCACCTCTGGTAGCACAACC |
| #304 | reverse | CCTTTAGAAGTGACCAGATGTTCTGTGTGGTACTCT |
| | | C |
| #305 | reverse | CCATGAGAAGTGACCAGATGTTCTGTGTGGTACTCT |
| | | C |

TABLE 4
Oligonucleotides used for PCR-amplifications

| Amplicon | oligonucleotides | template |
|---|---|---|
| FGA-ORF, wt, isoform α-E, | #1 | “FGA-Bio-His” |
| without stop-codon | #2 | |
| FGA-ORF, wt, isoform α-E, | #1 | “FGA-Bio-His” |
| with stop-codon | #68 | |
| FGA-ORF, wt, isoform α | #198 | pCMV6-XL4-FGA_new |
| | #201 | |
| FGA-ORF, K432H mutagenesis | #302 | pcDNA4/TO/FGA_new |
| | #304 | |
| FGA-ORF, 5 × KtoH mutagenesis | #303 | pcDNA4/TO/FGA_new |
| | #305 | |
| FGB-ORF, wt, isoform 1 | #3 | “FGB” |
| | #4 | |
| FGG-ORF, wt, isoform gamma-B | #5 | “FGG” |
| | #6 | |
| FGG-ORF, K88S, K111S, K382S, | #5 | “FGG mutation” |
| K399S, isoform gamma-B | #6 | |
| FGG-ORF, wt, isoform gamma-A | received ORF already in pcDNA3 | |
| FGG-ORF, K88H, K111H, K382H, | received ORF already in pcDNA3 | |
| K399H, isoform gamma-A | | |
| FGG-ORF, K88A, K111A, K382A, | received ORF already in pcDNA3 | |
| K399A, isoform gamma-A | | |
| FGB-operon (incl. CMV promoter | #7 | pcDNA3-FGB |
| and bGH poly(A)signal) | #8 | |
| FGG-operon (incl. CMV promoter | #9 | pcDNA3-FGG, |
| and bGH poly(A)signal) | | pcDNA3-FGG_mut, |
| | #10 | pcDNA3-FGG_HisM4, |
| | | or pcDNA3-FGG_AlaM3 |

### Gibson Assembly

For the insertion of the different mutations into the γ-chain, a part of the DNA sequence of the γ-chain was purchased from ThermoFisherScientific (Waltham, USA). In order to perform Gibson assembly pairs of primer for both the vector containing the γ-chain as well as the synthesized gene were designed (FGG Insert Fw: 5′-gagaacaaga ccagcgaagt gaa-3′; FGG Insert Rv: 5′-cttcatggtg gttttcttca tgc-3′: FGG Vector Fw: 5′-gtacagcatg aagaaaacca cca-3′; FGG Vector Rv: 5′-cttcacttcg ctggtcttgt tct-3′). The amplified cDNA was purified using PCR purification kits (E.Z.N.A® Kits, Omega Bio-tek, Inc., Norcross, USA) and inserted into the vector pcDNA3 and transformed into E. coli dH5a. Successful cloning was verified by sanger sequencing (Eurofins Genomics, Ebersberg, Germany).

### Building of Expression Plasmids

All cloning steps were performed via SLIC, using PCR-amplified inserts and vectors opened with restriction enzymes. First, the open reading frame (ORF) for fibrinogen (Fbg) α-chain was cloned into the Xbal-opened pcDNATM4/TO/myc-His A vector, either without a stop-codon to incorporate the vector-encoded myc-His tag or with a stop-codon for untagged versions. The ORFs for fibrinogen β- and γ-chain have been cloned into the EcoRI-opened pcDNATM3 vector. In a second step, the FGB- and FGG-ORFs were PCR-amplified together with the surrounding CMV promoter and bGH poly(A)signals (using oligonucleotides #7 and #8, or #9 and #10, respectively) and inserted in a single SLIC-reaction into the pcDNA4/TO/FGA construct, opened by restriction with Sapl-enzyme (cuts in the vector backbone between the ColE1 origin of replication and the bleomycin resistance gene). Clones were screened by a combination of colony-PCRs and analytical restriction analyses and verified by sequencing all three ORFs.

### Cell Culture

ExpiCHO-S™ cells were cultured according to manufacturer's protocol. In short, cells were maintained in 125 mL flask in ExpiCHO™ Expression Medium at 37° C., 8% CO2 and 130 rpm without the use of antibiotics. For general maintenance cells were passaged every 3-4 days when a density of approximately 6×106 cells/mL was reached.

### Production of Recombinant Fibrinogen

Fibrinogen variants were expressed according to the user manual (Thermo Scientific, Waltham, USA). Briefly, ExpiCHO-S™ cells (Thermo Scientific, Waltham, USA) were cultured to a final density of 10×106 cells/mL. Plasmids encoding for the different fibrinogen variants (0.8 μg/mL of culture) as well as ExpiFectamine™ CHO (Thermo Scientific, Waltham, USA) were diluted in 5 mL of cold OptiPro™ medium (Thermo Scientific, Waltham, USA) and incubated for 5 min at RT. The ExpiFectamine™ CHO/DNA complex was transferred to the shaker flask and incubated at 37° C. and 8% CO2 and 130 rpm. After 24 h of incubation, ExpiFectamine™ CHO Enhancer and ExpiFectamine™ CHO Feed (Thermo Scientific, Waltham, USA) were added to the culture flask. The culture flasks were transferred to an incubator at 32° C. and 8% CO2 with shaking at 130 rpm. This step was repeated after 4 days of incubation. After another incubation step of up to 7 days at 32° C. and 8% CO2 and 130 rpm cells were harvested and centrifuged at 3000 rpm for 15 minutes.

For the production of recombinant fibrinogen (rFbg) with the addition of UK 370106, the inhibitor was added to the medium to a final concentration of 25 μM. Every day, cells were centrifuged at 1100 rpm for 4 min. The supernatant was removed, EDTA was added to a final concentration of 2 mM, and it was stored at −80° C. until further use. Purification was done as described before with the addition of 2 mM EDTA to every buffer used during the process.

Purification of fibrinogen Recombinant fibrinogen WT and mutant were purified from the supernatant of ExpiCHO-S cells. After filtration of the supernatant using a microfiltration membrane (Filtropur S 0.2 μM, Polyethersulfon-Membran, Sarstedt, Nuembrecht, Germany) the solution containing the recombinant protein was purified by ion exchange chromatography using a FPLC system with a Capto MMC column (GE Healthcare Äkta Explorer, Life sciences, Freiburg, Germany) equilibrated in 20 mM phosphate buffer (pH 7) containing 150 mM NaCl. The protein was eluted from the column with a linear gradient using a 200 mM arginine buffer (pH 8.5) with 500 mM NaCl. After buffer exchange to a 10 mM arginine buffer (pH 8.5) containing 150 mM NaCl recombinant fibrinogen was purified in a second step using size exclusion chromatography (Hiload 16/600 Superdex 200 PG, GE Healthcare, Life sciences, Freiburg, Germany) with 10 mM arginine buffer (pH 8.5) containing 150 mM NaCl. Protein concentration was determined using BCA or Bradford assay according to manufacturer's manual. The recombinant protein was stored at −80° C.

### SDS-Page

Purified proteins and conjugates were analyzed using Tris-glycine SDS-PAGE either with or without the addition of β-mercaptoethanol as described before.58 Gels were stained with Coomassie Brilliant Blue G250 and documented using FluorChem FC2 imaging system (Protein Simple, Santa Clara, USA).

### Formation of Fibrin Gels

Lyophilized fibrinogen (SigmaAldrich) was dissolved in ddH2O and dialyzed against 10 mM Tris/HCl, 150 mM NaCl (pH 7.5) over night at RT. Gelation of fibrinogen (20 mg/mL) was started by the addition of thrombin (Merck) (1 U/mL), factor Xllla (fibrogamin®, CSL Behring) (50 mU/mL) as well as CaCl2 (10 mM). 100 μL of the fibrinogen solution were transferred to a 96 well plate and incubated at RT for 2 h. Afterwards gels were stored at 4° C. upon further experiments.

### Plasmin Digest

In order to analyze plasmin digestion of mutated restriction sites, 100 μL of human plasmin (Merck) (0.5 U/mL) were added to 100 μg of Fbg and rFbg (Ala and His) and incubated for 24 h at 37° C. and 130 rpm in order to ensure complete cleavage. Afterwards, samples were stored at −80° C. For the analysis of the time-dependent cleavage site determination and quantification of plasmin, fibrin gels (20 mg/mL) were incubated with different plasmin concentrations ranging from 0.005 mU/mL-0.5 U/mL. Samples were taken at different timepoints from 1 min to 24 h. PMSF (1 mM) was added to the sample and heated at 95° C. for 10 minutes to ensure plasmin inactivation. Samples were stored at −80° C.

### In-Gel Digest High-Resolution Mass Spectrometry

Samples were diluted with Nupage Sample buffer™. In a next step dithiothreitol (DTT) was added to the samples to a final concentration of 50 mM for the reduction of disulfide bonds. After denaturation of the proteins at 70° C. for 10 min. iodoacetamide (IAA) was added to a final concentration of 120 mM and the samples were incubated for 20 min in the dark. SDS-PAGE was performed (vide supra) and the band of interest was cut out of the gel and stored at −80° C. until further use.

For in-gel digestion, the excised gel slices were destained with 30% acetonitrile, shrunk with 100% acetonitrile, and dried in a vacuum concentrator.

Trypsin digest was performed overnight at 37° C. in 0.05 M NH4HCO3 (pH 8), using 0.1 μg of protease per slice. Peptides were extracted from the gel slices with 5% formic acid.

ProAlanase digest was performed by incubating the gel-slice with 0.1% Formic acid, containing 0.2 μg of ProAlanase for 1.5 h. Peptides were extracted as above.

### Mass Spectrometry Analysis

Sample preparation and in-solution digest:

Proteins were digested either with plasmin alone (vide supra) or with plasmin and ProAlanase (ProAla) (Promega) with slight differences in handling. For all digests, all samples (WT/HIS/ALA) were brought to a concentration of 0.2 μg/μL in 50 mM tris buffer. For “plasmin only” samples were immediately purified (see below). For ProAla (Promega), 50 μL of 8M Guanidinium Hydrochloride were added to 10 μg (50 μL) of protein stock solution and briefly heated to ensure denaturation. To enable specific ProAla digest the solution was acidified using 900 μL of 32 mM HCl. After pH was checked to be in the range of 1-1.5, ProAla was added (1:50 ProAla:Protein; 0.25 μg), proteolysis was allowed to continue for 2.5 h. Afterwards, pH was set to slightly basic by addition of 0.5-1 mL of 2 M Tris buffer. In the next step, the samples were immediately purified.

Prior to purification samples were reduced and alkylated as follows:

Tris(2-carboxyethyl)phosphine (TCEP) was added to a final concentration of 20 mM. Samples were heated to 60° C. for 5 min and IAA was added to a final concentration of 40 mM during cooling. The samples were allowed to fully cool in the dark for 20 min. Immediately after, the samples were loaded on 1 mL Strata X33 syringe columns (10 mg bed; Phenomenex, Torrance, CA). For all steps only gravitational flow was used. The columns were activated with 3 mL 100% ACN and then washed with 3 mL 100% deionized water. Then the sample was added. Afterwards, the column was washed with 5 mL 0.4% formic acid in ddH2O. Elution was performed with 0.5 mL 0.4% formic acid in 80% ACN. The eluted samples were frozen in liquid nitrogen and freeze dried. For analysis, samples were resuspended in 50 μL 0.2% formic acid, 2% acetonitrile and used for LC MS/MS analysis immediately.

### Screening of Inhibitors for Fbg Expression

ExpiCHO-S cells were cultured to a density of 6×106 cells/mL. Afterwards, cells were centrifuged for 4 min at 1000 rpm at RT and the supernatant was collected. Commercially available fibrinogen (SigmaAldrich) was mixed with the supernatant to a final concentration of 3.3 mg/mL and a volume of 200 μl. The mixture incubated at 37° C. and 750 rpm and samples were taken at 0, 24 and 48 h. For the screening the different inhibitors were added to the mixture (final concentrations: PMSF: 1 mM; Aprotinin: 50 μg/mL; GM6001: 25 μM; EDTA: 4 mM; 1,10 Phenanthroline: 1 mM; UK 370106: 100 μM). As control fibrinogen was incubated in 10 mM Tris/HCl, 150 mM NaCl (pH 7.5) under the same conditions. For the visualization of fibrinogen degradation, 20 μL of the mixtures were analyzed using SDS-PAGE.

### LC-MS/Data Analysis

NanoLC-MS/MS analysis were performed on an LTQ-Orbitrap Velos Pro (Thermo Fisher Scientific, Darmstadt, Germany) equipped with a PicoView Ion Source (New Objective, Littleton, USA) and coupled to an EASY-nLC 1000 (Thermo Fisher Scientific, Darmstadt, Germany). Peptides were loaded on a precolumn (trap column, 2 cm×150 μM ID) packed with 3 μm C18 ReproSil and then eluted to capillary columns (30 cm×150 μm ID) self-packed with ReproSil-Pur 120 C18-AQ, 1.9 μm and separated with either a 30, 60, 120-minute linear gradient from 3-30% acetonitrile and 0.1% formic acid and a flow rate of 500 nL/min.

Gradients were used as follows: 30 min gradients were used for in-gel digested samples, 60 min gradients for time resolved plasmin cleavage site determination and quantification, 120 min gradients for all other in solution digests.

MS scans were acquired in the Orbitrap analyzer with a resolution of 30,000 at m/z 400, MS/MS scans were acquired in the Orbitrap analyzer with a resolution of 7500 at m/z 400 using HCD fragmentation with 30% normalized collision energy. A TOP5 data-dependent MS/MS method was used; dynamic exclusion was applied with a repeat count of 1 and an exclusion duration of 7 seconds; singly charged precursors were excluded from selection. Minimum signal threshold for precursor selection was set to 50,000. Predictive automatic gain control (AGC) was used with AGC target a value of 1×106 for MS scans and 5×104 for MS/MS scans. Lock mass option was applied for internal calibration in all runs using background ions from protonated decamethylcyclopentasiloxane (m/z 371.10124).

### Data Analysis

Data analysis was performed using PMI-Byos (Protein Metrics inc., Cupertino United states). Data was searched against databases containing all human proteins (uniport reference proteome) for quality control (data not shown), when the target protein was identified searches detailed below were performed against custom databases containing either the WT sequences of α-, β- and γ-chain of fibrinogen, or in the case of γ-chain analysis only the γ-sequence. For determination of successful expression after inhibitor addition, the isoform sequence (P02671-2|FIBA_HUMAN Isoform 2) from uniport was used. Samples were searched using PMI-Preview (Protein Metrics inc., Cupertino United states) first, and suggested modifications were included in the main search. In all cases decoys were added. Results were filtered and only peptide matches with a scoring of >100 were considered.

Settings for individual searches varied and were as follows:

### Determination of Cleavage Efficiency in Mutants

All datafiles were searched in batches of 3 (separated by protease used for digest), with the settings described below.

Plasmin Only digest (γ):

Precursor Tolerance of 15 ppm, fragment mass tolerance of 20 ppm, digestion C-terminal of K (non-specific), modifications are concurrent with the PMI-Byos recommendations for sequence variant analysis workflows (two rare and common modifications allowed).

Plasmin and ProAlanase digest (γ):

Precursor Tolerance of 15 ppm, fragment mass tolerance of 20 ppm, digestion C-terminal of A and P (non-specific), modifications are concurrent with the PMI-Byos recommendations for sequence variant analysis workflows (two rare and common modifications allowed).

In addition to that, a “quality control” assessment of the experiments was performed. Both sets of digests were searched against a database containing all three fibrinogen chains. Settings were as follows: 5 ppm precursor and 20 ppm fragment tolerance, digestion C-terminal of APKR, set to semi-specific with up to 5 missed cleavages (data not shown). Modifications were drastically reduced compared to the gamma chain searches. Data was filtered as above.

α-Chain in-Gel Digest

### Tryptic Digest:

A custom database of all three chains, their mutants and other proteins was used. Settings: 5 ppm precursor and 20 ppm fragment tolerance, digestion C-terminal of R/K, fully specific with up to 2 missed cleavages allowed, one common and one rare modification allowed.

### ProAlanase Digest:

A custom database of all three chains, their mutants and other proteins was used. Settings: 5 ppm precursor and 20 ppm fragment tolerance, digestion C-terminal of P/A, non-specific with up to 2 missed cleavages allowed, one common and one rare modification allowed.

α-Chain Expression with Protease Inhibitors in-Gel Digest

The database contained isoform 2 of the alpha chain and the other two chains. Settings: 5 ppm precursor and 20 ppm fragment tolerance, digestion C-terminal of R/K, fully specific with up to 3 missed cleavages allowed, three common and two rare modifications allowed.

### Time Resolved Cleavage Site Determination and Quantification

This search was performed using MaxQuant (V1.6.17.0) using a database containing all three Fbg chains, and in addition plasminogen, prothrombin, Factor FXIIIa and angiotensin peptide, common contaminants were added. Datafiles of 6 consecutive timepoints were searched in batch. The instrument specific standard settings was used, specific trypsin digest but allowing up to 7 missed cleavages to account for differences between plasmin and trypsin, match between runs feature, allowed for up to 5 modifications. Allowed modifications were: Oxidation (M), Acetyl (N-term), Deamidation (NQ), Phospho (STY) and fixed Carbamidomethyl (C). Bioinformatic analysis was performed using Perseus (V 4.1.3). The results were filtered as follows: removing contaminants, at least one intensity >0. Then all intensities were normalized to the angiotensin intensity of the respective datafile. This excluded the last two timepoints, as the angiotensin intensity was too low to be confirmed. Then information for each of the respective Fbg chains was extracted. For each chain separately and per timepoint, intensities of all peptides, either regarding their start or end position, were summed up. The sum of intensities for all peptides starting or ending at e certain cleavage site, allowed us to develop a timepoint, not dependent on a single peptide, but on a certain cleavage site.

### Results

### Design of Recombinant Fibrinogen for Improved Stability Towards Plasmin

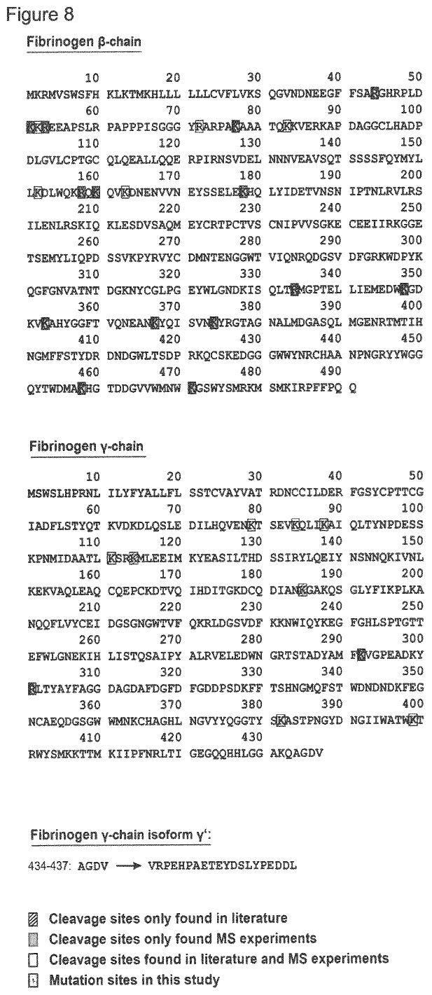

Fibrinolysis has been studied for a long time and multiple cleavage sites of plasmin are described in all chains (α-, β- and γ-chain). Furthermore, plasmin cleavage sites are distributed and located in different parts all over of the quaternary structure of fibrinogen. In order to investigate the influence of mutations (AA exchange) and the influence of their location with respect to the complex structure on proteolytic degradation, plasmin cleavage sites in different parts of the quaternary structure of fibrinogen were chosen. Mutation sites are located in the α-helical domain as well as the random coil domain (known as D-domain44; FIG. 1A). It was chosen to insert mutations only in the gamma-chain over distributing mutations throughout all chains. The goal was to facilitate an improved degradation stability of the complex by keeping one composite chain intact, while keeping changes to the minimum. The gamma chain was selected because it has the smallest number of cleavage sites compared to alpha and beta chains, as well as facilitating crosslinking via factor Xllla. The 4 different cleavage sites, either in the α-helical or D-domain also allowed us to study their influence on the structure of fibrinogen. The mutation sites are QLI(K)AIQ; ATL(K)SRM (both alpha helical domain); TYS(K)AST; ATW(K)TRW (both D-domain) (FIG. 1A, B, FIG. 7-8). The ideal substrate of plasmin contains either an arginine (Arg, R) or a lysine (Lys, K) residue at Position P1. In order to test the influence of AA exchange in these sequences on plasmin cleavage, the 4 different sequences were synthesized changing K to serine (Ser, S) as a polar AA as proof of principle by using SPPS. After purification, peptides were incubated with plasmin and cleavage efficiency was analyzed using HPLC. Peptide sequences including S instead of K showed a significant reduction in cleavage efficiency compared to the original sequences. After this proof of principle, the aforementioned mutations were introduced in the γ-chain by using site-directed mutagenesis. Furthermore, in order to test the influence of different fibrinogen forms two different expression plasmids were built. One containing Aα-, Bβ- and γ-chain (recombinant fibrinogen, rFbg) and one containing αE-, Bβ- and γ′-chain (recombinant fibrinogen isoforms, rFbgi).

### Production of Recombinant Fibrinogen

The production of recombinant fibrinogen was done using eukaryotic protein expression in CHO cells. The construction of expression plasmid pcDNA4-TO-FBG was done using ligation-independent cloning (SLIC; FIG. 9). The plasmid encodes for all 3 chains, α, β and γ-chain. All chains are set in row and are under control of the human cytomegalovirus (CMV) promoter, a promoter that is frequently used for eukaryotic protein expression due to high levels of production. Bovine growth hormone (bGH) polyadenylation signal was added to the end of each gene to assure transcription termination (FIG. 2). Expression plasmids were built for both, wild type (WT) and a variant carrying all 4 mutations.

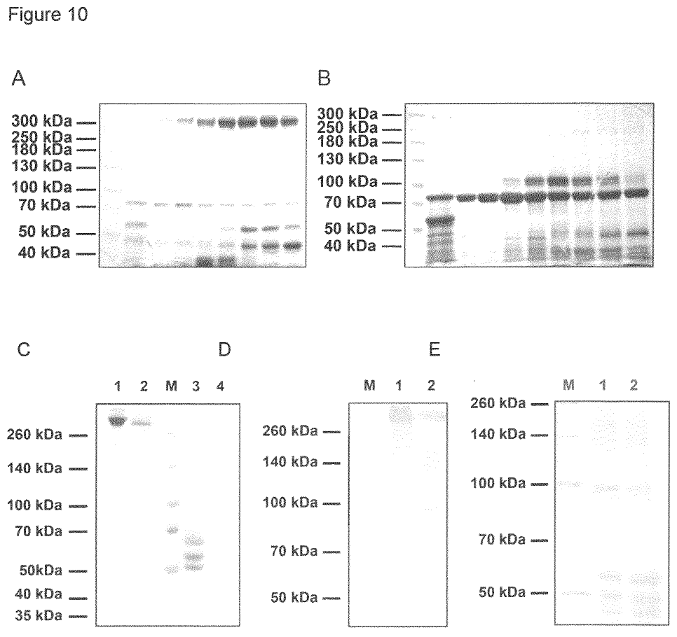

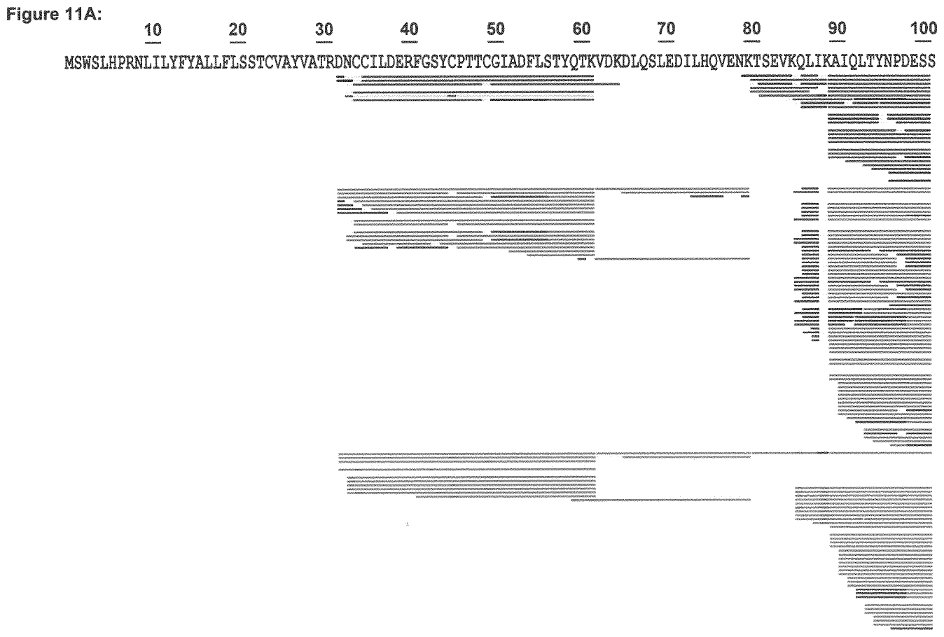

CHO cells were transfected with the expression plasmid and incubated for up to 12 days. Afterwards, the cell suspension was centrifuged, and the supernatant was collected. Purification of the recombinant protein was done by anion exchange chromatography (AiEx) using multimodal functionalities on a Capto MMC column for high volume throughput and enhanced binding capacity. After the expression of rFbgi, the non-reduced SDS-PAGE analysis showed a band at over 300 kDa for WT which can be attributed to the native hexamer form of fibrinogen. However, no high molecular band was visible for the expression for Ser-fibrinogen indicating that the introduction of S instead of K at the 4 mutations sites lead to either degradation or truncation of the γ-chain and therefore prevents fibrinogen production and assembly (FIGS. 10A&B). It is described in literature that S can in rare cases act as a helix breaker in secondary protein structures. As two of the 4 mutations are located in the middle of the α-helical part of fibrinogen, these mutations could lead to the interruption of the helix and therefore disrupt the formation of the quaternary structure.

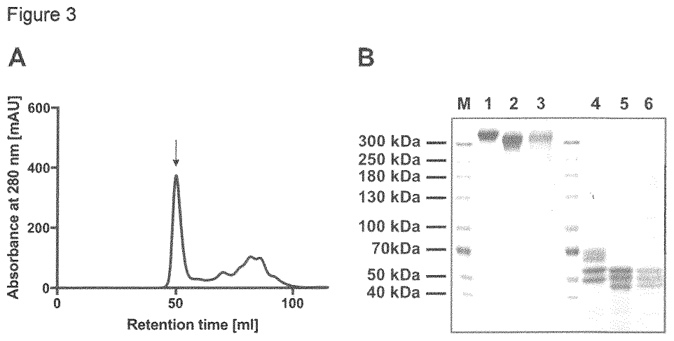

In order to mimic the physicochemical properties of the target AAs histidine (His, H) and alanine (Ala, A) were chosen as substitutes for K. A is known to be abundant in α-helices and is regarded the most stabilizing AA for helices. H is also known to stabilize α-helices due to hydrogen bonding with the CO-group in the helix backbone. Furthermore, histidine mimics the positive charge of the natural lysine residue and therefore might stabilize (or at least not disrupt) the structure of fibrinogen. After the insertion of the new mutations into the plasmid using gibson assembly and SLIC, the new mutants of both rFbg and rFbgi were produced in CHO cells and showed a high purity after size exclusion chromatography (SEC, FIG. 3A, B). Non-reduced SDS-PAGE analysis showed a band for all 4 variants (His-rFbgi, Ala-rFbgi, rFbg, His-rFbg, Ala-rFbg) at a high molecular weight of over 300 kDa (FIG. 3B, FIG. 10 D, E). This suggests that the insertion of these mutations did not impair protein expression and the fibrinogen hexamer was built.

### Structural Analysis of Recombinant Fibrinogen

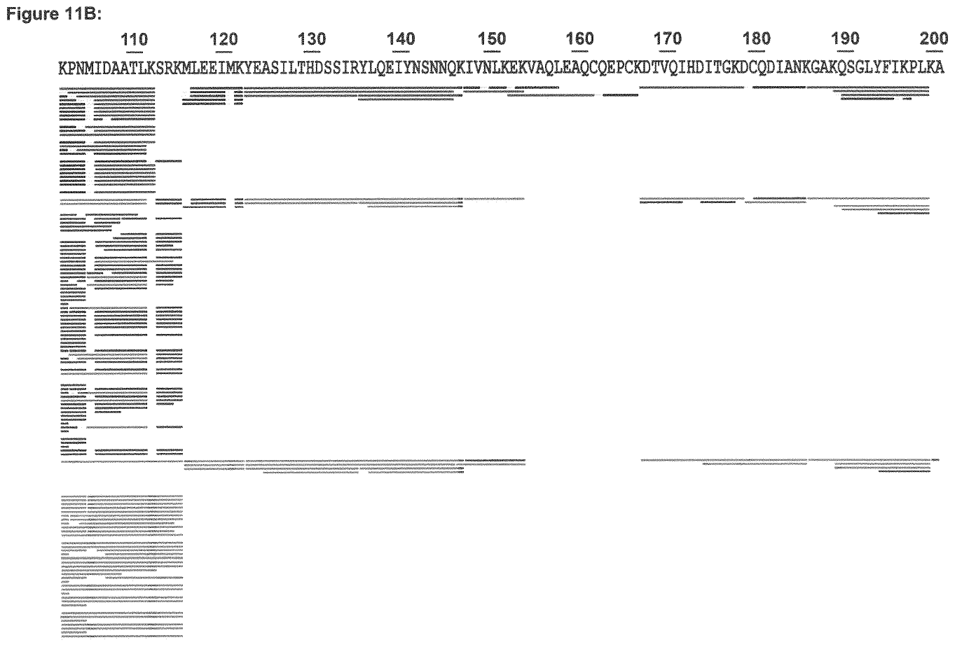

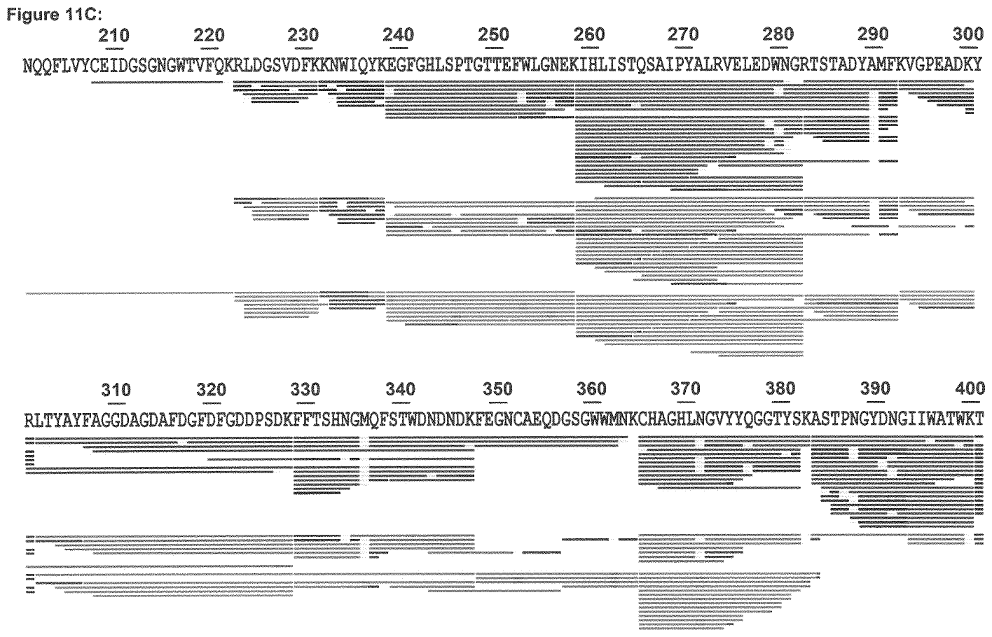

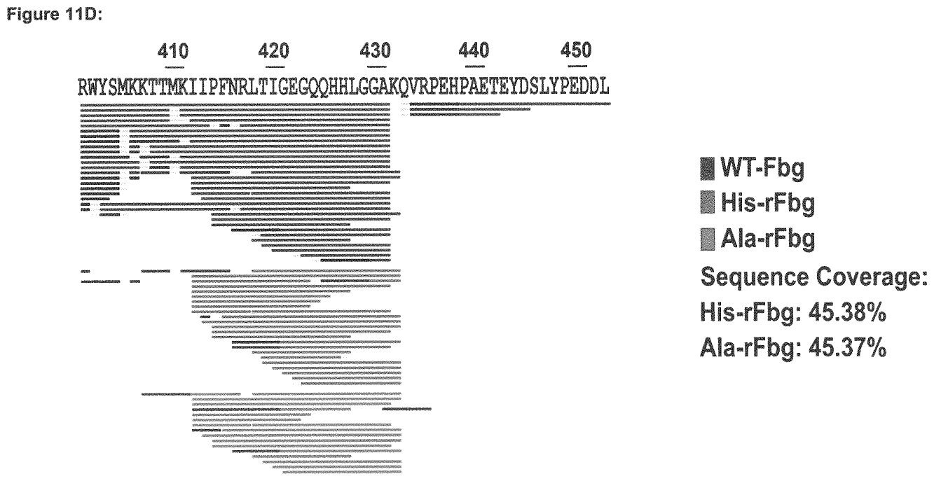

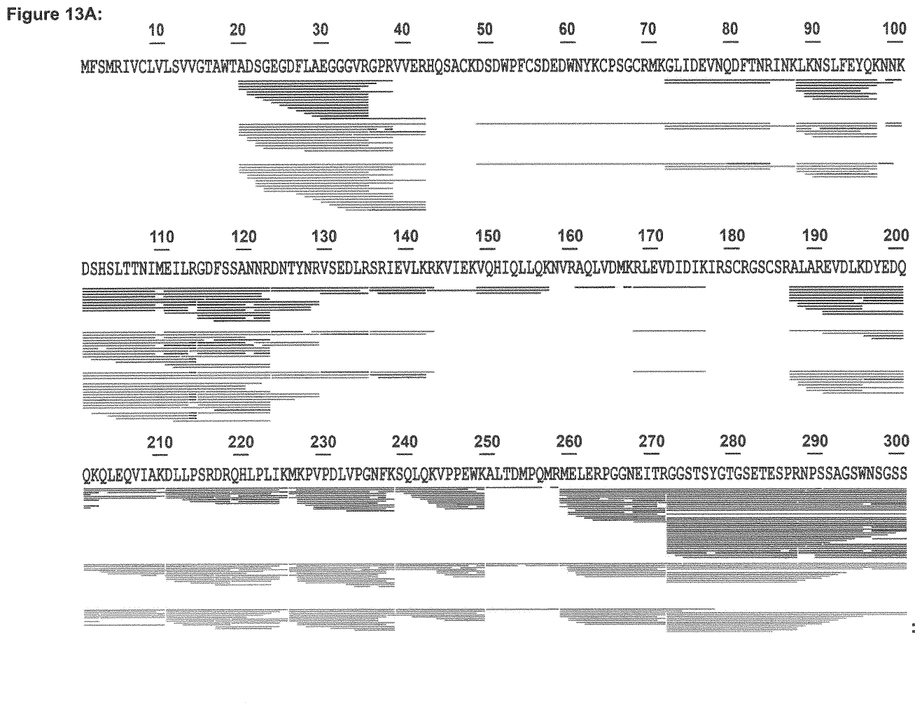

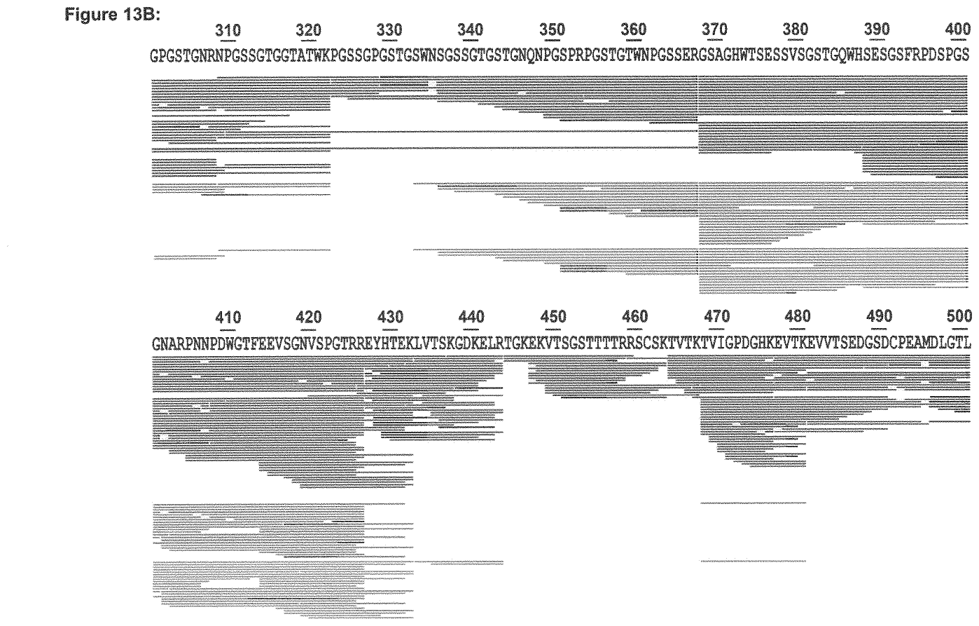

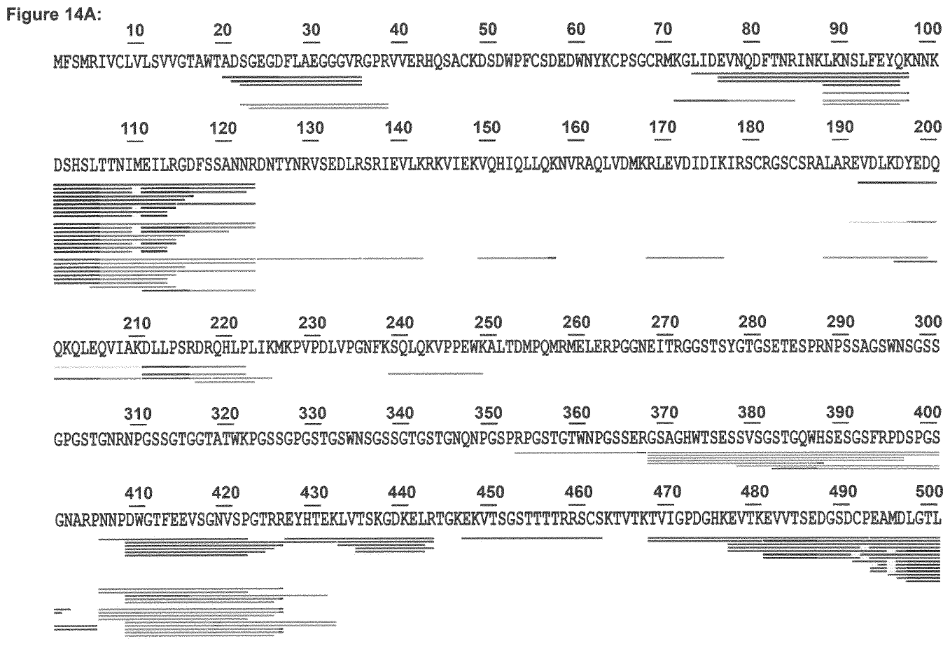

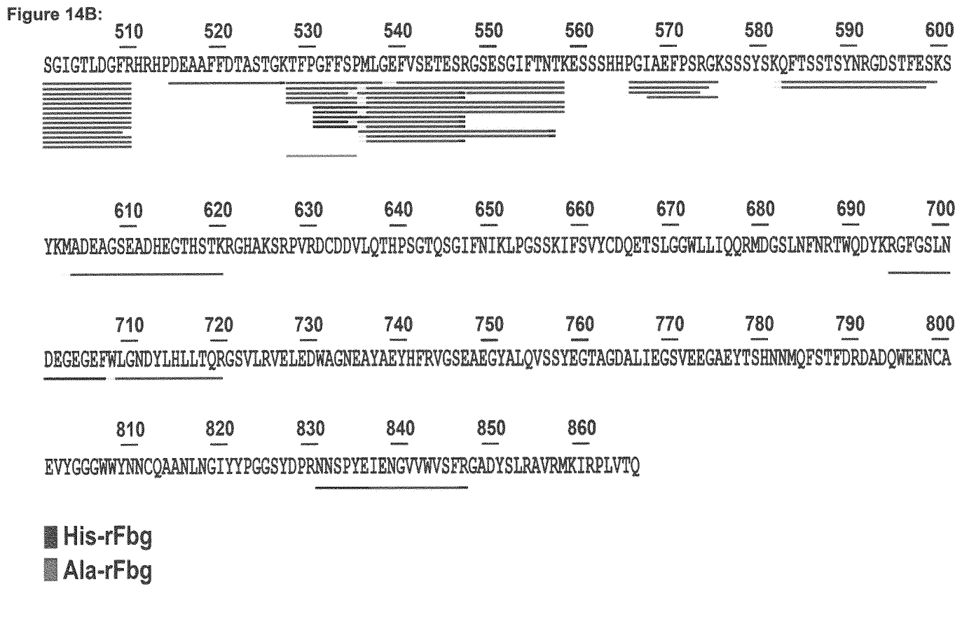

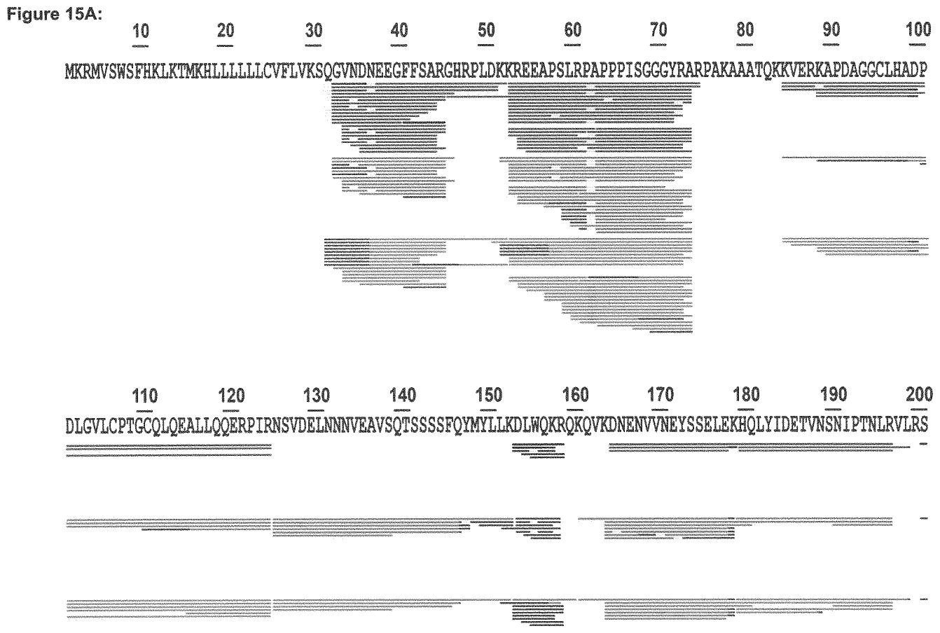

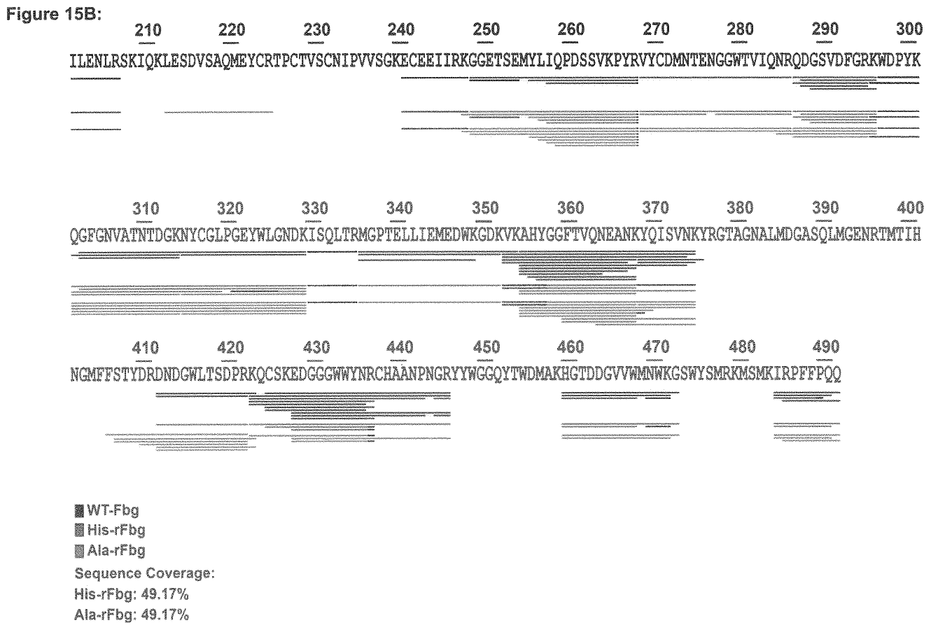

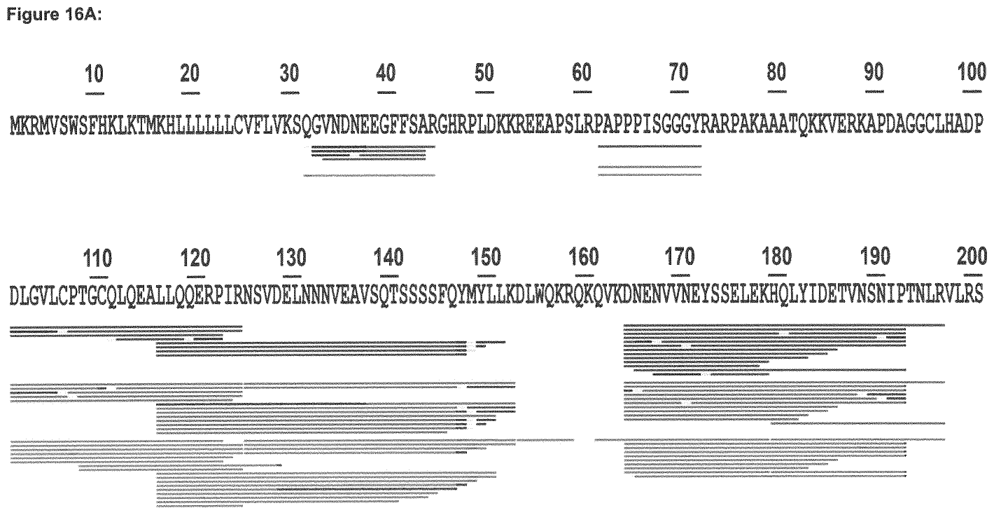

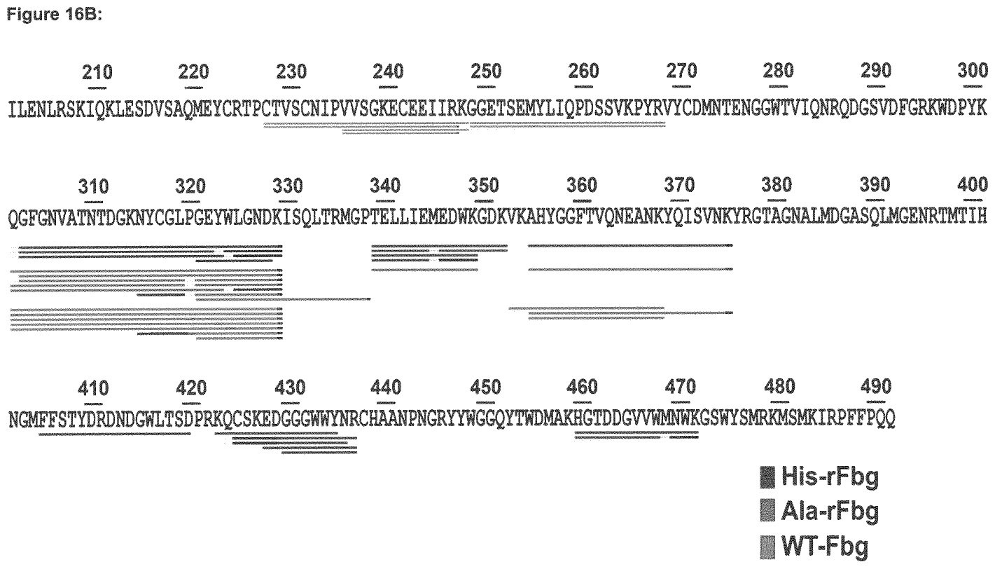

To further verify the structure of the recombinant fibrinogen mutants, reduced SDS-PAGE was performed. Both β- and γ-chain were visible on the gel with molecular weights of around 52 kDa and 46.5 kDa as well as the native form at over 300 kDa (FIG. 3B, FIG. 10 C-E). Furthermore, the presence and sequence of the different chains was confirmed by high resolution ESI-MS (FIG. 11-16). This shows that the introduction of the different mutations did not impair gene expression, the γ-chain could be produced and that the general approach can be used in the future. However, the band of the α-chain (α-chain: 66.5 kDa; αE: 93 kDa) was not visible on the SDS-PAGE under reducing conditions. Instead, another band emerged at around 44 kDa for both rFbg and rFbgi indicating a truncation of the α-chain during the production or purification process (FIG. 3B, FIG. 10 C-E). Furthermore, the native fibrinogen band was slightly lower to the control also indicating a truncation of the protein (FIG. 3B).

The fibrinogen α-chain, especially the C-terminus, is generally more susceptible to proteolytic degradation. CHO cells a known to secret proteases during cell culture which can lead to proteolytic degradation of the alpha chain. However, every CHO cell line has its own unique protease expression pattern which makes it difficult to identify the specific protease responsible. For the optimization of the approaches, it was focused on the rFbg (Aα-, Bβ- and γ-chain) from this point on because this form of fibrinogen is generally used as part of fibrin glues.

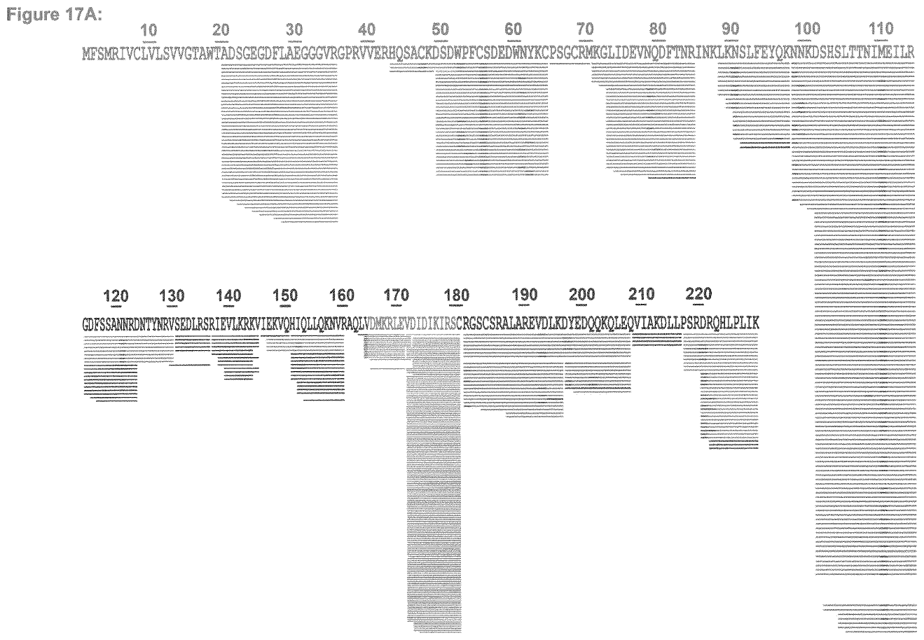

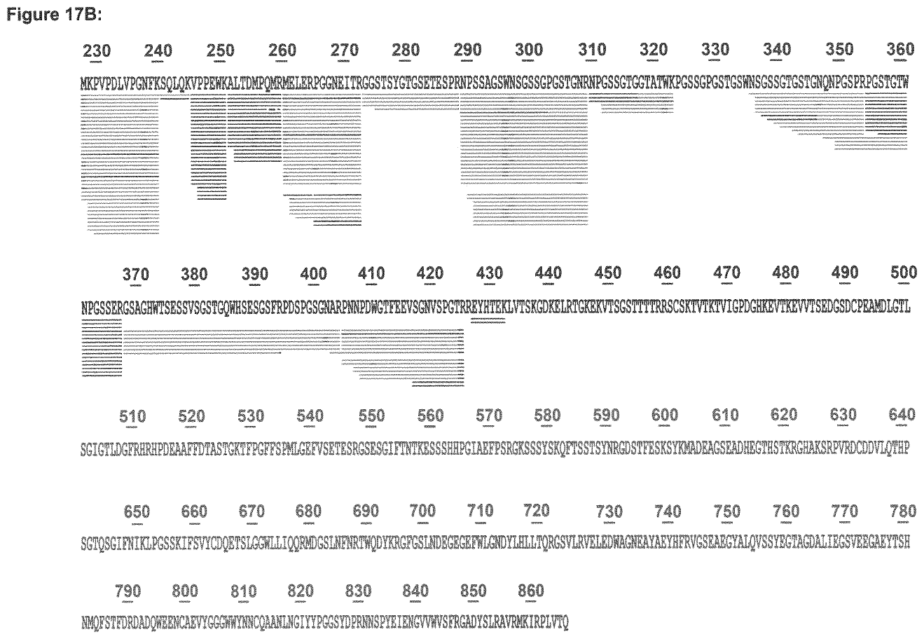

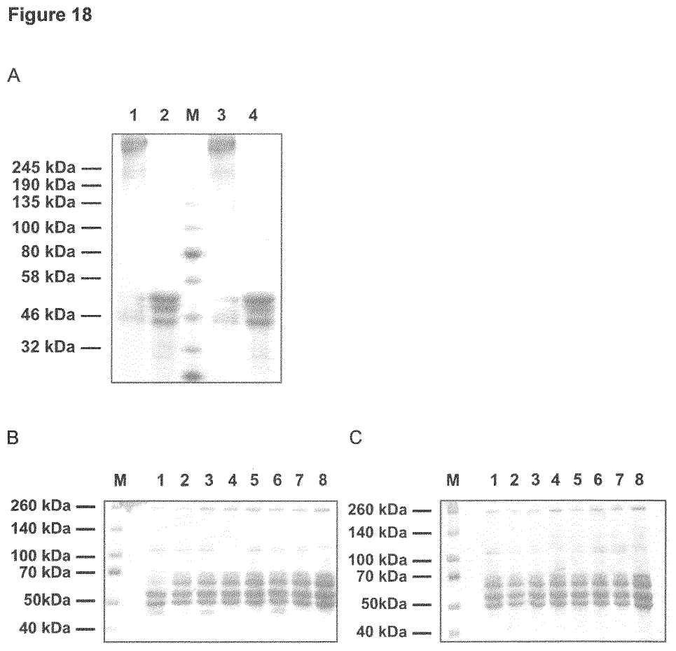

In order to verify that the new band at around 44 kDa is a truncated form of the α-chain and to locate the exact site of truncation, an in-gel digestion for a mass spectrometry characterization of the fragment was performed. High resolution ESI-MS analysis showed a high alignment with the native α-chain and a truncation of the chain at K432 (FIG. 4A, B, C, FIG. 17), confirmed by both, tryptic and ProAlanase digest (data not shown). This results also matches with the molecular weight of the band and the AA sequence missing. Degradation of the α-chain during expression is often described in literature as a result of protease cleavage in the supernatant. It has been described before, that the addition of the serine protease inhibitor aprotinin during CHO cell expression did inhibit proteolytic degradation of the fibrinogen α-chain.⁵¹ In order to limit this degradation, the same approach as described previously was applied and K432 to H was exchanged in the α-chain gene cassette to inhibit proteolytic cleavage of the mutation site (rFbg432). In addition, an expression plasmid with all K inside 20 AA up/downstream of the cleavage site K432 (K432, K437, K440, K446, K448: rFbg5xKtoH) exchanged to H to prevent the emergence of a new cleavage site due to evolutionary pressure was built. However, SDS-PAGE analysis after CHO cell protein expression of both His-rFbg432 and His-rFbg5xKtoH revealed that the α-chain is still degraded during the process (FIG. 18 A). Other enzymes that have been reported to degrade the α-chain are matrix metalloproteinases (MMPs). Analysis on the influence of different MMPs on fibrinogen showed a degradation pattern resembling the ones of both rFbg and rFbgi and a potential cleavage site at K432 (e. g. MMP 2, 3, 12, 14).

Therefore, different serine-protease (suggested in literature) and MMP inhibitors on their influence on the α-chain degradation were screened, to find a suitable compound for the inhibition of α-chain degradation. Commercially available human Fbg was incubated with the supernatant taken from the CHO cells used for the expression and incubated the protein at 37 C for several days. The analysis using SDS-PAGE showed the same degradation pattern for Fbg as seen during the expression. The truncated α-chain is also visible after the addition of the serin protease inhibitor aprotinin which could be an explanation why the inserted mutation at 432 did not have an impact on the degradation (FIG. 18 B, C). However, an intact α-chain was detected even a couple of days after the addition of either EDTA, 1,10 Phenantroline or the MMP inhibitor UK 370106 (FIG. 4D, FIG. 18 B, C). This shows that the degradation of rFbg during CHO cell expression is likely the result of an overexpression of MMPs in the supernatant and a subsequent cleavage of the α-chain.

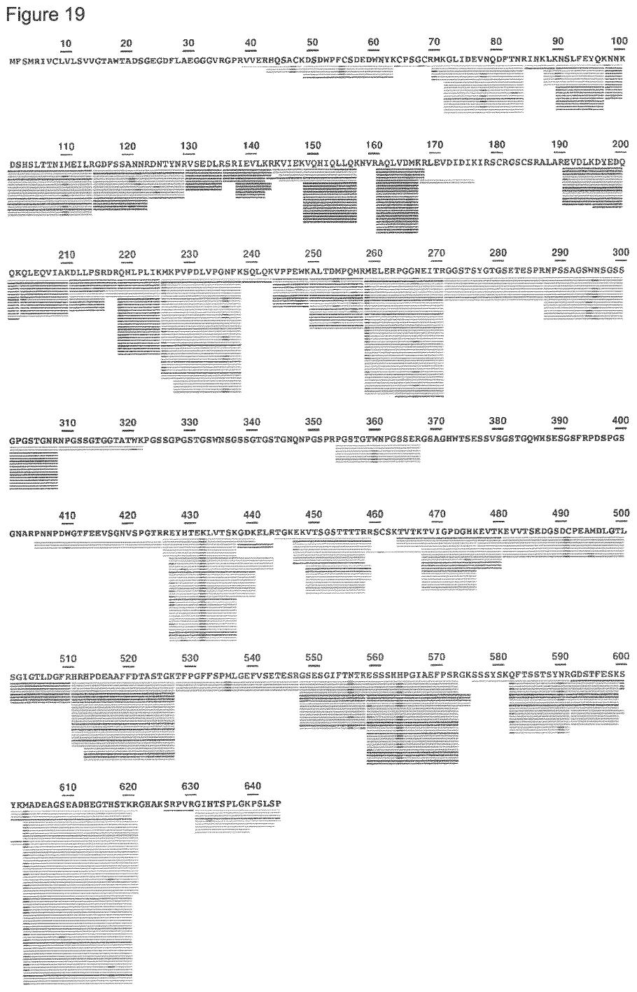

Hence, His-rFbg432 with the addition of 25 μM of UK 370106 was expressed. Reduced SDS-PAGE analysis showed three bands at around 68 kDa, 52 kDa and 46.5 kDa similar to the ones in commercially available Fbg indicating that the addition of the UK 370106 lead to the production of a fully intact α-chain (FIG. 4E). The slight difference in molecular weight of the α-chain compared to the control could be due to a potential difference in the complex glycosylation pattern of rFbg. The sequence of the α-chain was verified using high resolution ESI-MS (FIG. 19).

### Analysis of Plasmin Digest of the γ-Chain of Recombinant Fibrinogen Mutants

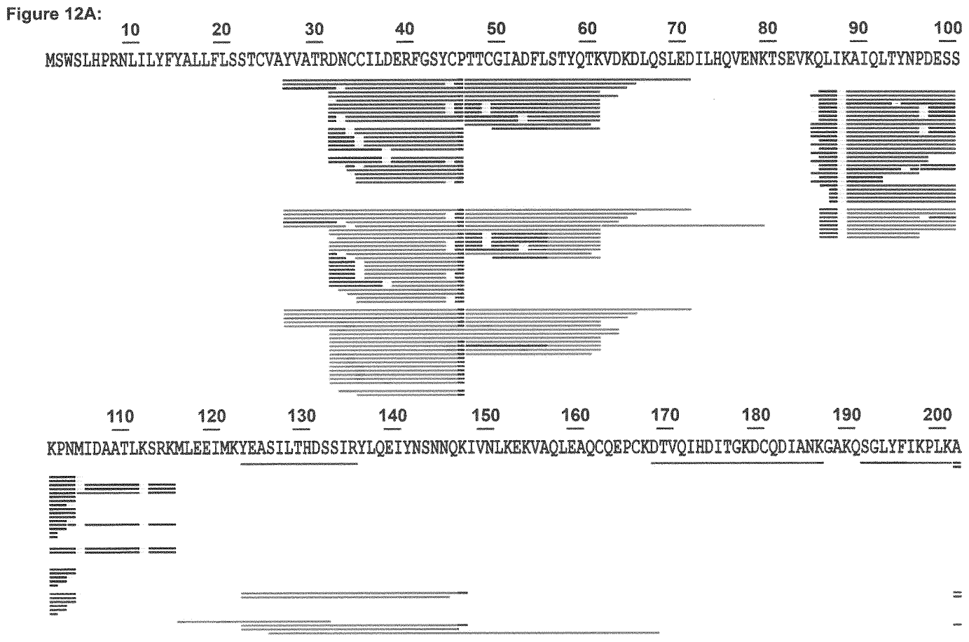

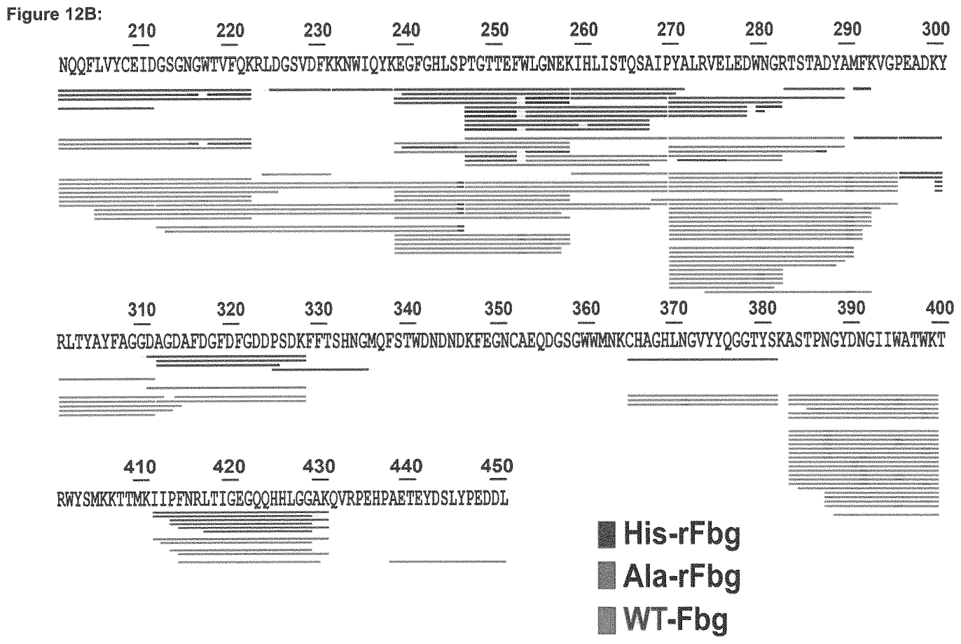

The α-chain plays an important part in the gelation process of fibrin. Due to the truncated α-chain gel formation with rFbg was not possible, as the production of intact complex (compare above) was still ongoing. Therefore, plasmin digests in solution with WT as well as both Ala- and His-rFbg mutants was performed to investigate if the inserted mutations are limiting proteolytic efficiency of plasmin at the respective cleavage sites. Plasmin was added to the fibrinogen solution (1 mg/mL) to a final concentration of 0.5 U/mL and the mixture was incubated for 24 h to ensure complete cleavage of fibrinogen. Using high-resolution ESI-MS the 4 different cleavage sites K88, K111, K382, K399 in the WT could be detected. However, no cleavage was detected in either of the two mutants for all 4 mutations showing that the AA exchange from K to A or H does limit proteolytic efficiency of plasmin in Fbg (FIG. 5, FIG. 11-12, FIG. 20). At some positions both cleaved and non-cleaved peptides were detected, however no cleavage did contain a mutation. This might be due to residual WT-Fbg peptides either on the column (carryover) or through residual WT-Fbg at any point in the workup. The present inventors believe that this cannot be an expression or cell line artifact since CHO cells do not produce Fbg naturally and the expression plasmid only contains the mutated form of the γ-chain.

### Time Dependent Analysis of Plasmin Digest of Fibrinogen

After proving that the exchange of AA exchange from K to either H or A does impair plasmin mediated degradation of the γ-chain, it was analyzed which cleavage sites are cut in intact fibrin gels during the early phase of fibrinolysis. These sites could be potential targets for site-directed mutagenesis in the future in order to stabilize and limit fibrinogen degradation directly at the start. For this, fibrin gels (Sigma Aldrich, 20 mg/mL) were cast, and a plasmin solution (0.005 mU/ml) was added on top of the gels and incubated for up to 2 h. At different time points the supernatant was collected and plasmin was inactivated by the addition of PSMF and heating. Afterwards, the solutions were purified using a C18 gravity column (Strata X, Phenomenex, Torrance, CA) and 100 fmol of ProteoMass™ Angiotensin II MALDI-MS Standard (Sigma-Aldrich, Steinheim, Germany) were added as an internal standard to each sample in order to quantify the cleavage of plasmin restriction sites. The time course of plasmin cleavage was analyzed using high resolution ESI-MS. For the analysis, the specificity of plasmin described in literature (cleavage C-terminal of R/K, similar to trypsin) was used. However, an untargeted search revealed several non-tryptic cleavage sites. Thus, plasmin apparently cleaves both C- and N-terminal of R/K. In addition, some recurrent interfaces were observed showing AAs other than R/K in P1 and P1′ positions.

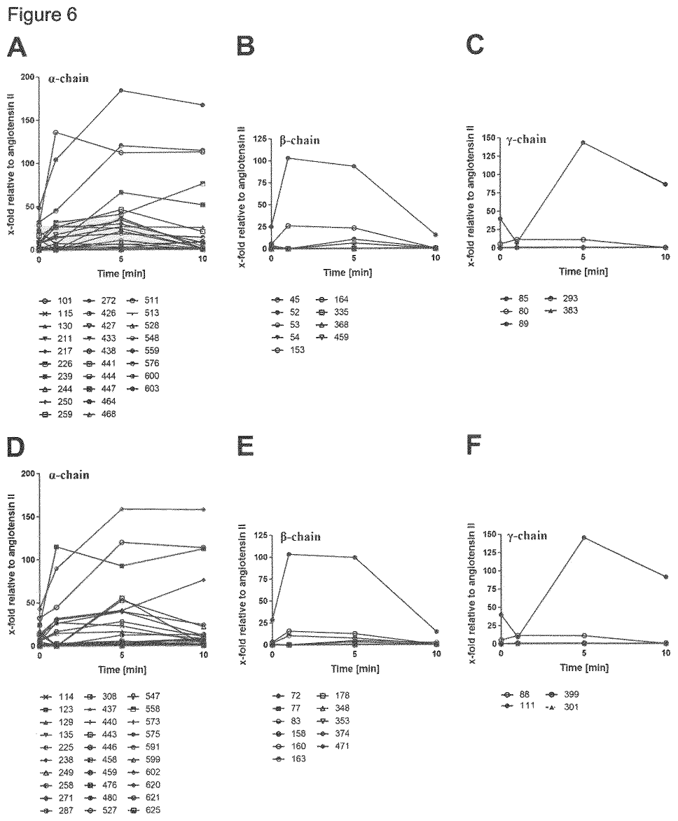

The analysis shows that cleavage of fibrin gels in the earlier stages is most prominent within the α-chain which is consistent with reports from literature. The data supports the reports that cleavage begins in the αC region of fibrinogen with the most prominent cleavage at positions 602 (K|MADE) and 620 (K|RGHA). Next, cleavage sites further down the αC region are cleaved leading to the complete removal of this part of fibrinogen (e. g. positions 527 (K|TFPG), 510 (R|HRHP), 225 (K|MKPV) and 238 (K|SQLQ)). At the same time, cleavage in the α-helical region of the α-chain begins (e. g. positions 100 (K|DSHS) and 123 (R|DNTY)) (FIG. 6A, D). However, the present inventors were able to detect a multitude of different cleavage sites with this approach that were not mentioned in the literature before (FIGS. 7-8). Furthermore, this approach enables a much more sensitive readout and analysis compared to methods such as HPLC and SDS-PAGE. In accordance with literature cleavage of the β-chain starts at the N-terminus highlighted by positions 52 (K|REEA) and 72 (R|ARPA) (FIG. 6B, E). Subsequently, the α-helical part between fragment D and E is cleaved by plasmin (e. g. 152 (K|DLWQ) and 160 (K|QVKD)) (FIG. 6B, E).

Strikingly, only one additional cleavage site was found in this time-resolved assay for γ-chain (FIG. 6C, F), supporting the initial hypothesis for the use of the γ-chain for the insertion of mutations. With only 5 cleavage sites detected during the early stages of plasmin degradation, less mutations are needed to potentially protect a complete chain in the fibrin complex.

### CONCLUSION

The fast resorption of fibrin sealants due to plasmin cleavage is still a major problem in hemorrhage control. However, there are only few approaches that have been studied over the past decades. Here, a novel approach using molecular engineering to limit plasmin-induced cleavage of fibrinogen is reported. Plasmin cleavage sites in both helical and random coil domains of fibrinogen were exchanged using site directed mutagenesis. The γ-chain was successfully produced in CHO cells, indicating that the insertion of mutations in different structures of the protein does not impact its folding or expression. Mass spectrometry analysis showed that plasmin cleavage of those sites was restricted after the AA proving that the AA exchange at plasmin restriction sites limits the cleavage by enzyme. In addition to that, a process for the production of rFbg in ExpiCHO-S™ cells was developed by screening and subsequent use of different MMP inhibitors during expression. Furthermore, a time resolved screening protocol for the elucidation of plasmin cleavage sites for a potential AA exchange for further generations of fibrinogen variants was developed. The approach for the production of a molecular engineered fibrinogen, described herein, could be used in the future to generate a new generation of fibrin sealants with an improved stability towards plasmin degradation. By varying both the number of mutations at plasmin cleavage sites as well as their location in the quaternary structure of the protein, fibrin gel resorption could potentially be fine-tuned for specific application.

### Example 2

### Materials and Methods

### Turbidity Assays:

Turbidity experiments were conducted on a Tecan Infinite M Plex plate reader at 37° C. A 96-well plate with a final volume of 100 μl per well was used and was experiments were performed in triplicate. The Thrombin-catalyzed fibrin polymerization of fibrinogen (0.1 mg/mL) was initiated by the addition of thrombin (0.05 U/mL), factor Xllla (Fibrogammin®, CSL Behring; 50 mU/ml) as well as CaCl₂ (10 mM). The change in turbidity at 350 nm was followed over time (0-3600 sec).

### Cross-Linking of Fibrin:

To examine cross-linking of fibrin, fibrinogen (0.1 mg/mL) was incubated at 37° C. with FXIIIa (0.5 U/mL) and human-thrombin (final concentration, 0.05 U/mL) in Tris-buffer and CaCl₂ (10 mM). The reactions were stopped at various times by addition of an equal volume of sodium dodecyl sulfate (SDS) sample buffer with 2-mercaptoethanol and incubation (5 min) at 90° C. Samples were separated on SDS-polyacrylamide gel electrophoresis (SDS-PAGE) and stained with Coomassie brilliant blue.

### Results

### Design and Production of Recombinant Fibrinogen for Improved Stability During Mammalian Cell-Based Protein Expression

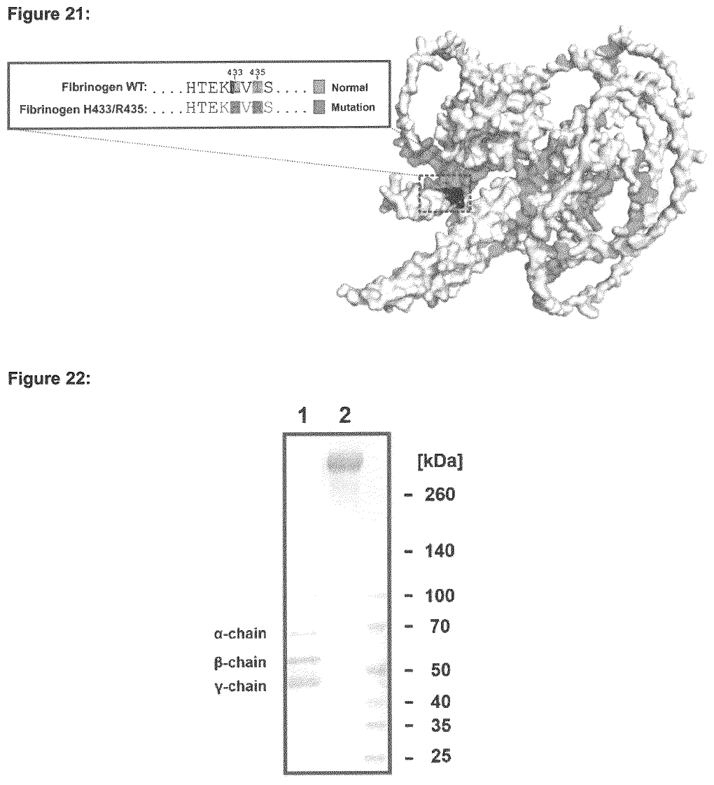

Degradation of fibrinogen during mammalian protein expression has been long recognized and several cleavage sites of MMPs are described in all chains.^{50, 51} Specifically, the α-chain appears to be affected by degradation, impairing the functionality of the resulting fibrinogen.⁵² Based on the MS analysis of the degraded α-chain (see above), the mutations into the target sites of the MMPs were incorporated. The aim was to achieve improved degradation stability of the complex during cell-based protein expression by maintaining the α-chain intact while keeping modifications to a minimum. The mutation sites are in spatial proximity within the sequence ⁴²⁹HTEK(H)V(R)S⁴³⁶ (FIG. 21). To test the influence of different forms of fibrinogen, an expression plasmid was produced. The plasmid consists of Aα, Bβ- and γ-chain (His-rFbgH433R435), with mutations only in the Aα- and γ-chain.

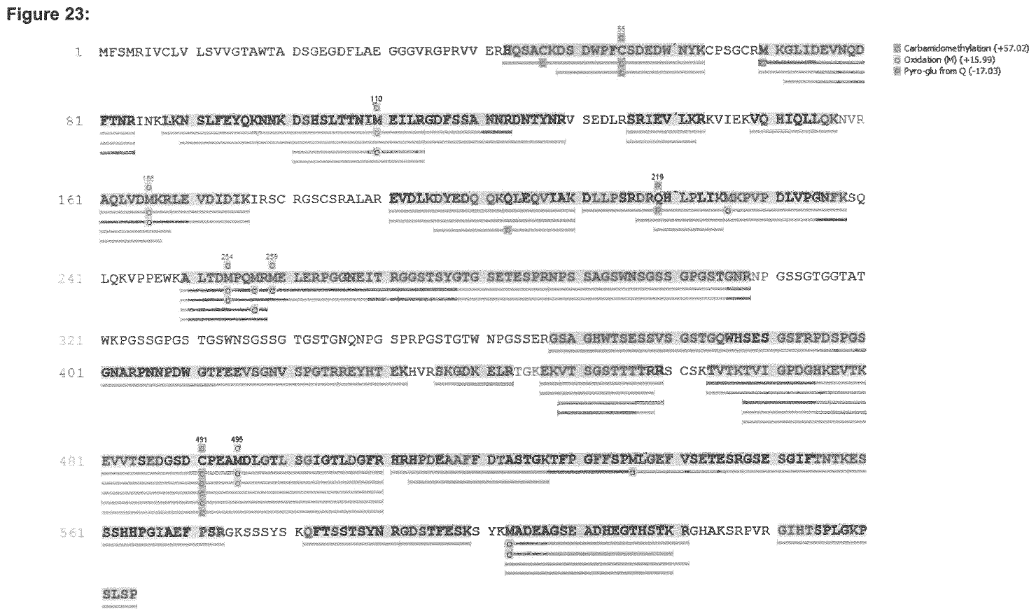

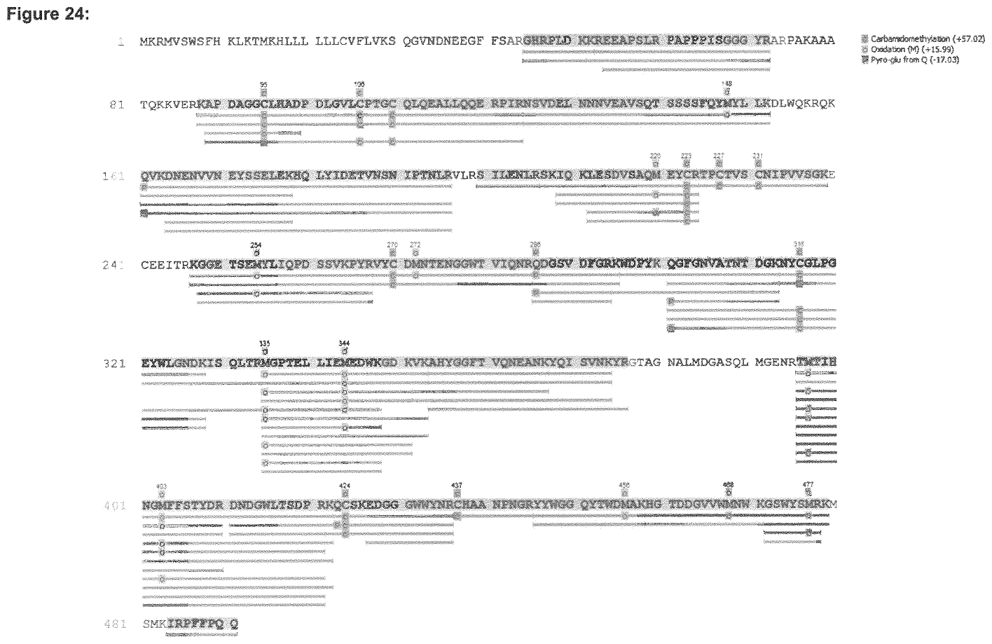

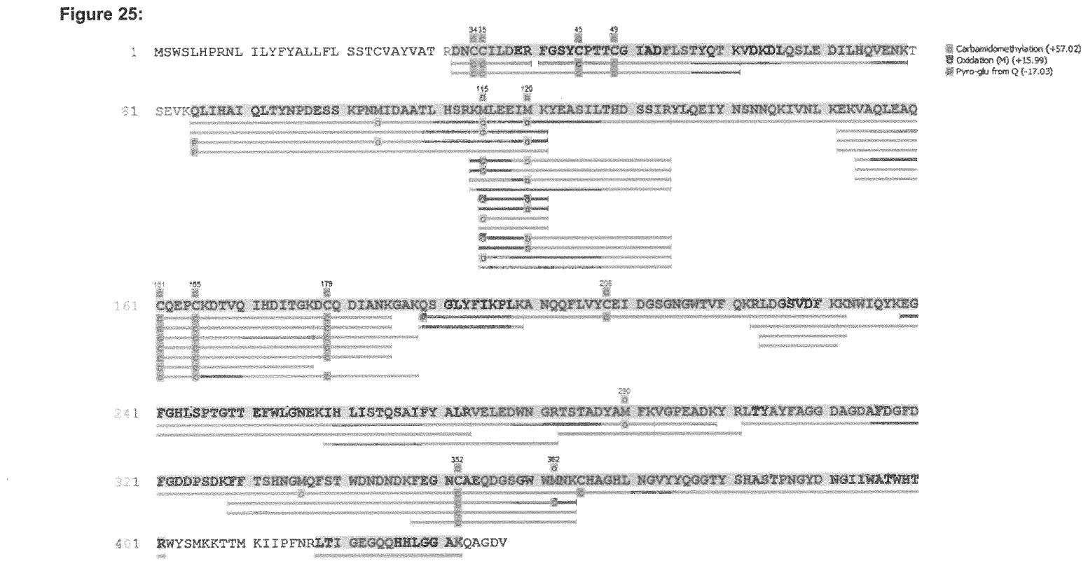

After insertion of the new mutations into the plasmid by Gibson assembly and SLIC, the new mutant was produced in CHO cells and showed high purity after size exclusion chromatography. Non-reducing SDS-PAGE analysis revealed a band with a high molecular weight of over 300 kDa (FIG. 22). This indicates that the insertion of these mutations did not affect the protein expression and that the fibrinogen hexamer was correctly assembled. Furthermore, the reducing SDS-PAGE and in-gel digestions (FIG. 22-25) detected the presence of all three chains. However, the degradation product of the α-chain was not detectable in this variant. Thus, confirming that mutation in the α-chain enhances stability during recombinant expression in CHO cells.

### Time-Dependent Analysis of Fibrin Polymerization

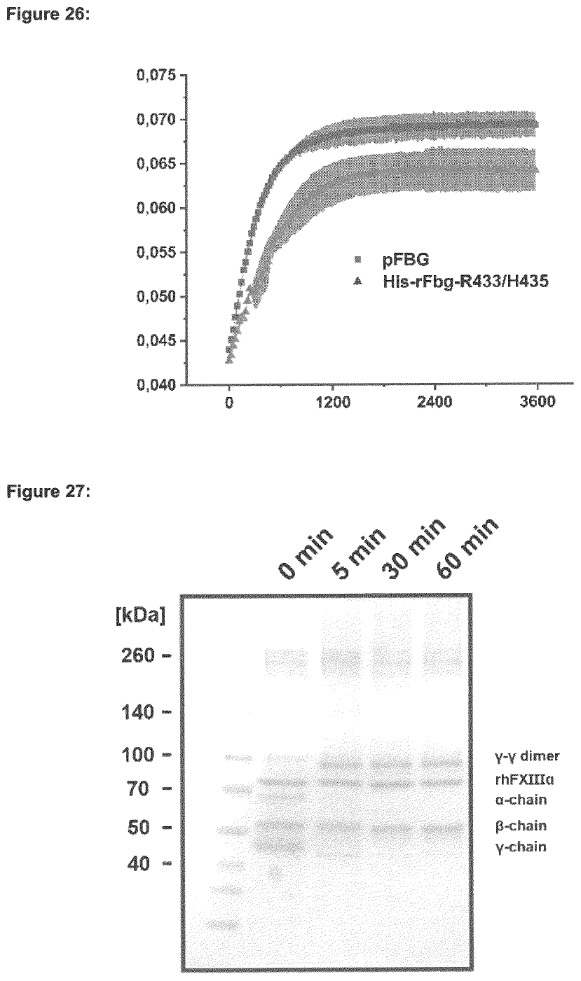

After proving that the exchange of AA exchange does impair the proteolytic degradation effects of the α-chain during expression, the ability of the mutant to form fibrin fibers was investigated. As shown in FIG. 26, thrombin-induced fibrin polymerization of His-rFbgH433R435 was slightly reduced compared with control (pFbg), with a similar lag time and comparable absorption. To further assess the functionality of the of the fibrinogen variant, FXIIIa-catalyzed cross-linking of γ-chains was performed (FIG. 27). Here, the cross-linked γ-γ-dimer bands of rFbgH433R435 were visible after 5 min, indicating rapid lateral linkage. The intensity increased with longer incubation, whereas the intensity of the γ- and Aα-chain bands decreased after each incubation period. However, the experiment demonstrated that the mutant forms functional fibrin gels and thus would be potentially suitable as a wound adhesive.

### REFERENCES

1. Meara, J. G.; Leather, A. J. M.; Hagander, L.; Alkire, B. C.; Alonso, N.; Ameh, E. A.; Bickler, S. W.; Conteh, L.; Dare, A. J.; Davies, J.; Mérisier, E. D.; El-Halabi, S.; Farmer, P. E.; Gawande, A.; Gillies, R.; Greenberg, S. L. M.; Grimes, C. E.; Gruen, R. L.; Ismail, E. A.; Kamara, T. B.; Lavy, C.; Lundeg, G.; Mkandawire, N. C.; Raykar, N. P.; Riesel, J. N.; Rodas, E.; Rose, J.; Roy, N.; Shrime, M. G.; Sullivan, R.; Verguet, S.; Watters, D.; Weiser, T. G.; Wilson, I. H.; Yamey, G.; Yip, W., Global Surgery 2030: evidence and solutions for achieving health, welfare, and economic development. The Lancet 2015, 386 (9993), 569-624.
2. Vollset, S. E.; Goren, E.; Yuan, C. W.; Cao, J.; Smith, A. E.; Hsiao, T.; Bisignano, C.; Azhar, G. S.; Castro, E.; Chalek, J.; Dolgert, A. J.; Frank, T.; Fukutaki, K.; Hay, S. I.; Lozano, R.; Mokdad, A. H.; Nandakumar, V.; Pierce, M.; Pletcher, M.; Robalik, T.; Steuben, K. M.; Wunrow, H. Y.; Zlavog, B. S.; Murray, C. J. L., Fertility, mortality, migration, and population scenarios for 195 countries and territories from 2017 to 2100: a forecasting analysis for the Global Burden of Disease Study. Lancet 2020, 396 (10258), 1285-1306.
3. Ramirez Manuel G, C. G. F., Ramirez Manuel A, The Economic Burden of Bleeds and Transfusions in Selected Surgeries: A Retrospective Multi Center Analysis from the Us Perspective. Am J Biomed Sci & Res. 2019, 2(5).
4. Reportlinker. Global Medical Adhesives and Sealants Industry. https://www.reportlinker.com/p05478477/Global-Medical-Adhesives-and-Sealants-Industry.html?utm_source=GNW (accessed Nov. 8, 2021).
6. Berrevoet, F.; de Hemptinne, B., Use of Topical Hemostatic Agents during Liver Resection. Digestive Surgery 2007, 24 (4), 288-293.
7. Aldrighetti, L.; Pulitanò, C.; Arru, M.; Catena, M.; Finazzi, R.; Ferla, G., “Technological” approach versus clamp crushing technique for hepatic parenchymal transection: a comparative study. J Gastrointest Surg 2006, 10 (7), 974-9.
8. Martinez-Mier, G.; Esquivel-Torres, S.; Alvarado-Arenas, R. A.; Ortiz-Bayliss, A. B.; Lajud-Barquin, F. A.; Zilli-Hernandez, S., Liver resection morbidity, mortality, and risk factors at the departments of hepatobiliary surgery in Veracruz, Mexico. Rev Gastroenterol Mex 2016, 81 (4), 195-201.
9. Girardis M., B. S., Marietta M., Severe bleeding in critical care. Gullo A. (eds) Anaesthesia, Pain, Intensive Care and Emergency A.P.I.C.E. Springer, Milano 2006.
10. Doci, R.; Gennari, L.; Bignami, P.; Montalto, F.; Morabito, A.; Bozzetti, F.; Bonalumi, M. G., Morbidity and mortality after hepatic resection of metastases from colorectal cancer. Br J Surg 1995, 82 (3), 377-81.
11. Christensen, M. C.; Krapf, S.; Kempel, A.; von Heymann, C., Costs of excessive postoperative hemorrhage in cardiac surgery. J Thorac Cardiovasc Surg 2009, 138 (3), 687-93. 12. Colson, P. H.; Gaudard, P.; Fellahi, J. L.; Bertet, H.; Faucanie, M.; Amour, J.; Blanloeil, Y.; Lanquetot, H.; Ouattara, A.; Picot, M. C., Active Bleeding after Cardiac Surgery: A Prospective Observational Multicenter Study. PLoS One 2016, 11 (9), e0162396.
13. Biancari, F.; Brascia, D.; Onorati, F.; Reichart, D.; Perrotti, A.; Ruggieri, V. G.; Santarpino, G.; Maselli, D.; Mariscalco, G.; Gherli, R.; Rubino, A. S.; De Feo, M.; Gatti, G.; Santini, F.; Dalén, M.; Saccocci, M.; Kinnunen, E. M.; Airaksinen, J. K.; D'Errigo, P.; Rosato, S.; Nicolini, F., Prediction of severe bleeding after coronary surgery: the WILL-BLEED Risk Score. Thromb Haemost 2017, 117 (3), 445-456.
14. Kim, Y. J.; Shin, S. K.; Lee, H. J.; Chung, H. S.; Lee, Y. C.; Park, J. C.; Hyung, W. J.; Noh, S. H.; Kim, C. B.; Lee, S. K., Endoscopic management of anastomotic leakage after gastrectomy for gastric cancer: how efficacious is it? Scandinavian Journal of Gastroenterology 2013, 48 (1), 111-118.
15. Matz, D.; Teuteberg, S.; Wiencierz, A.; Soysal, S. D.; Heizmann, O., Do antibacterial skin sutures reduce surgical site infections after elective open abdominal surgery?—Study protocol of a prospective, randomized controlled single center trial. Trials 2019, 20 (1), 390.
16. Edmiston, C. E.; Krepel, C. J.; Marks, R. M.; Rossi, P. J.; Sanger, J.; Goldblatt, M.; Graham, M. B.; Rothenburger, S.; Collier, J.; Seabrook, G. R., Microbiology of Explanted Suture Segments from Infected and Noninfected Surgical Patients. Journal of Clinical Microbiology 2013, 51 (2), 417.
17. Golling, M.; Schaudt, A.; Mehrabi, A.; Mood, Z. A.; Bechstein, W. O., Clinical application of soft polyglycolic acid felt for hemostasis and repair of a lacerated liver: report of two cases. Surg Today 2008, 38 (2), 188-92.
18. Spotnitz, W. D., Fibrin Sealant: The Only Approved Hemostat, Sealant, and Adhesive—a Laboratory and Clinical Perspective. ISRN Surgery 2014, 2014, 203943.
19. FDA US Food and Drug Administration. Premarket approval (PMA) for Ethicon OMNEX surgical sealant; 2010.
20. FDA US Food and Drug Administration. Premarket approval (PMA) for DuraSeal dural sealant system; 2005.
21. Grau, A. E.; Durán, J. A., Treatment of a large corneal perforation with a multilayer of amniotic membrane and TachoSil. Cornea 2012, 31 (1), 98-100.
22. Caspers, M.; Maegele, M.; Fröhlich, M., Current strategies for hemostatic control in acute trauma hemorrhage and trauma-induced coagulopathy. Expert Rev Hematol 2018, 11 (12), 987-995.
23. Hartmann, J.; Klein, H. G., Supply and demand for plasma-derived medicinal products—A critical reassessment amid the COVID-19 pandemic. Transfusion 2020, 60 (11), 2748-2752.
24. Baxter Package Insert. Tachosil; 2010.
25. Johnson, E. J. a. Package Insert. Evarrest; 2012.
26. FDA, Initial U.S. Approval. Tisseel by Baxter Healthcare Corporation. 1998.
27. Corporation, B. H. Baxter Advanced Surgery—Tisseel.
28. Palta, S.; Saroa, R.; Palta, A., Overview of the coagulation system. Indian J Anaesth 2014, 58 (5), 515-523.
29. Kawamura, M.; Sawafuji, M.; Watanabe, M.; Horinouchi, H.; Kobayashi, K., Frequency of transmission of human parvovirus B19 infection by fibrin sealant used during thoracic surgery. Ann Thorac Surg 2002, 73 (4), 1098-100.
30. Mosesson, M. W., Fibrinogen and fibrin structure and functions. J Thromb Haemost 2005, 3 (8), 1894-904.
31. Kattula, S.; Byrnes, J. R.; Wolberg, A. S., Fibrinogen and Fibrin in Hemostasis and Thrombosis. Arterioscler Thromb Vasc Biol 2017, 37 (3), e13-e21.
32. Yuan, H.; Vance, K. M.; Junge, C. E.; Geballe, M. T.; Snyder, J. P.; Hepler, J. R.; Yepes, M.; Low, C.-M.; Traynelis, S. F., The serine protease plasmin cleaves the amino-terminal domain of the NR2A subunit to relieve zinc inhibition of the N-methyl-D-aspartate receptors. J Biol Chem 2009, 284 (19), 12862-12873.
33. Bini, A.; Wu, D.; Schnuer, J.; Kudryk, B. J., Characterization of stromelysin 1 (MMP-3), matrilysin (MMP-7), and membrane type 1 matrix metalloproteinase (MT1-MMP) derived fibrin(ogen) fragments D-dimer and D-like monomer: NH2-terminal sequences of late-stage digest fragments. Biochemistry 1999, 38 (42), 13928-36.
34. Henschen, A.; McDonagh, J., Chapter 7 Fibrinogen, fibrin and factor XIII. In New Comprehensive Biochemistry, Neuberger, A.; van Deenen, L. L. M., Eds. Elsevier: 1986; Vol. 13, pp 171-241.
35. Hudson, N. E., Biophysical Mechanisms Mediating Fibrin Fiber Lysis. Biomed Res Int 2017, 2017, 2748340.
36. Kollman, J. M.; Pandi, L.; Sawaya, M. R.; Riley, M.; Doolittle, R. F., Crystal Structure of Human Fibrinogen. Biochemistry 2009, 48 (18), 3877-3886.
37. Roberts, I. V.; Bukhary, D.; Valdivieso, C. Y. L.; Tirelli, N., Fibrin Matrices as (Injectable) Biomaterials: Formation, Clinical Use, and Molecular Engineering. Macromolecular Bioscience 2020, 20 (1), 1900283.
38. Soria, J.; Mirshahi, S.; Mirshahi, S. Q.; Varin, R.; Pritchard, L. L.; Soria, C.; Mirshahi, M., Fibrinogen αC domain: Its importance in physiopathology. Res Pract Thromb Haemost 2019, 3 (2), 173-183.
39. (prev. 9) Cantero, M.; Rojas, H.; Anglés-Cano, E.; Marchi, R., Fibrin γ/γ′ influences the secretion of fibrinolytic components and clot structure. BMC Molecular and Cell Biology 2019, 20 (1), 47.
40. Greenberg, C. S.; Miraglia, C. C.; Rickles, F. R.; Shuman, M. A., Cleavage of blood coagulation factor XIII and fibrinogen by thrombin during in vitro clotting. J Clin Invest 1985, 75 (5), 1463-70.
41. Riedel, T.; Suttnar, J.; Brynda, E.; Houska, M.; Medved, L.; Dyr, J. E., Fibrinopeptides A and B release in the process of surface fibrin formation. Blood 2011, 117 (5), 1700-1706.
42. Joo, J. Y.; Amin, M. L.; Rajangam, T.; An, S. S. A., Fibrinogen as a promising material for various biomedical applications. Molecular & Cellular Toxicology 2015, 11 (1), 1-9.
43. Solovieva, E. V.; Fedotov, A. Y.; Mamonov, V. E.; Komlev, V. S.; Panteleyev, A. A., Fibrinogen-modified sodium alginate as a scaffold material for skin tissue engineering. Biomed Mater 2018, 13 (2), 025007.
44. Spotnitz, W. D., Fibrin Sealant: Past, Present, and Future: A Brief Review. World Journal of Surgery 2010, 34 (4), 632-634.
45. Jackson, M. R., Fibrin sealants in surgical practice: An overview. Am J Surg 2001, 182 (2 Suppl), 1s-7s.
46. Sahni, A.; Odrljin, T.; Francis, C. W., Binding of basic fibroblast growth factor to fibrinogen and fibrin. J Biol Chem 1998, 273 (13), 7554-9.
47. (prev. 18) Taboada, G. M.; Yang, K.; Pereira, M. J. N.; Liu, S. S.; Hu, Y.; Karp, J. M.; Artzi, N.; Lee, Y., Overcoming the translational barriers of tissue adhesives. Nature Reviews Materials 2020, 5 (4), 310-329.
48. Keragala, C. B.; Medcalf, R. L., Plasminogen: an enigmatic zymogen. Blood 2021, 137 (21), 2881-2889.
49. Lorentz, K. M.; Kontos, S.; Frey, P.; Hubbell, J. A., Engineered aprotinin for improved stability of fibrin biomaterials. Biomaterials 2011, 32 (2), 430-8.
50. Redman, C., Xia, H., Fibrinolysis and Proteolysis 2000, 14, 198-205.
51. H. Sandberg, D. Lütkemeyer, S. Kuprin, M. Wrangel, A. Almstedt, P. Persson, V. Ek, M. Mikaelsson, Biotechnology and Bioengineering 2006, 95, 961-971.
52. O. Hiller, A. Lichte, A. Oberpichler, A. Kocourek, H. Tschesche, Journal of Biological Chemistry 2000, 275, 33008-33013.

1. An isolated recombinant fibrinogen comprising two fibrinogen alpha-chains, two fibrinogen beta-chains and two fibrinogen gamma-chains, having amino acid sequences derived from human fibrinogen; characterized in that
(a) in the fibrinogen alpha-chains, with reference to the respective positions in SEQ ID NO: 2, one or more lysine (K) and/or arginine (R) residues selected from the group consisting R38, R42, K97, K100, R114, R123, R129, R135, K210, R216, R218, K225, K227, K238, K243, K249, R258, R271, R287, R308, R353, R367, R425, R426, K432, K437, K440, R443, K446, K448, R458, R459, K463, K467, K476, K480, R510, R512, K527, R547, K558, R573, K575, K581, R591, K599, K602, K620, R621, K625, R687 and R847 are substituted or deleted; and/or
(b) in the fibrinogen beta-chains, with reference to the respective positions in SEQ ID NO: 3, one or more lysine (K) and/or arginine (R) residues selected from the group consisting of R44, K51, K52, R53, R60, R72, K77, K83, R87, K152, R158, K160, K163, K178, R285, K328, R334, K348, K353, K367, K374, K458 and K471 are substituted or deleted; and/or
(c) in the fibrinogen gamma-chains, with reference to the respective positions in SEQ ID NO: 4, one or more lysine (K) and/or arginine (R) residues selected from the group consisting of R40, K79, K84, K88, K111, K114, K185, K199, K238, K292, R301, K382 and K399 are substituted or deleted.

2. The isolated recombinant fibrinogen of claim 1, wherein
i. the one or more substituted or deleted K and R residues in the fibrinogen alpha-chains comprise, or consist of, one or more of R135, K210, K243, R287, R308, R425, R426, K432, K437, K440, K446, K448, R458, R459, K463, K467, K480, R512, R547, K558, R573, K575, R591, K599, K620, R621, K625 and combinations thereof; and/or wherein the one or more substituted or deleted K and R residues in the fibrinogen beta-chains comprise, or consist of, one or more of R44, K51, R53, K77, R158, K160, K178, R334, K348, K353, K367, K374, K458, K471, and combinations thereof; and/or wherein the one or more substituted or deleted K and R residues in the fibrinogen gamma-chains comprise, or consist of, K292, R301, or both; and/or
ii. wherein the one or more substituted or deleted K and R residues in the fibrinogen alpha-chains comprise, or consist of, one or more of R38, R42, K97, K100, R114, R123, R129 R135, R216, K225, K238, K249, R258, R271, R443, R510, K527 and K602; and/or wherein the one or more substituted or deleted K and R residues in the fibrinogen beta-chains comprise, or consist of, one or more of K52, R72, K83, K152 and K163; and/or wherein the one or more substituted or deleted K and R residues in the fibrinogen gamma-chains comprise, or consist of, one or more of K79, K84, K88, K111, K114, K185, K382 and K399, preferably K79, K88, K111, K382 and K399; and
iii. optionally, wherein all K and R residues indicated in i) and/or in ii) are deleted or substituted, preferably substituted.

3. The isolated recombinant fibrinogen of claim 1, wherein the one or more substituted or deleted K and R residues in the fibrinogen alpha-chains comprise, or consist of K100, R114, R123, K225, K238, K249, R258, R271, R287, R308, R425, R426, K432, R443, R458, K467, R510, K527, R547, K558, K575, K599, K602, K621 and combinations thereof; and/or wherein the one or more substituted or deleted K and R residues in the fibrinogen beta-chains comprise, or consist of, R44, K51, K52, R72, K83, K152, R158, K160, K163 and combinations thereof; and/or wherein the one or more substituted or deleted K and R residues in the fibrinogen gamma-chains comprise, or consist of, one or more of K79, K88, K111, K382, K399 and combinations thereof.

4. The isolated recombinant fibrinogen of claim 1, wherein the fibrinogen alpha-, beta-, and gamma-chains comprise only substitutions, and no deletions, of the K or R residues.

5. The isolated recombinant fibrinogen of claim 1, wherein the one or more K or R residues are substituted with amino acids or isotopes of these amino acids selected from the group consisting of alanine (A), histidine (H), N6-[(2-azidoethoxy)carbonyl]-L-lysine, propargyl-L-lysine, azidohomoalanine, trans-cyclooct-2-en-L-lysine, exo BCN-L-lysine, trans-cyclooct-4-en-L-lysine, pyrrolysine and pyrrolysine analogs, N^{ε}-cyclopentyloxycarbonyl-L-lysine, lysine-nitrobenzyl-oxycarbonyl-N^{ε}-L-lysine, O-methyl-L-tyrosin and analogs, methionine (M), isoleucine (I), leucine (L), phenylalanine (F), tryptophane (W), preferably selected from alanine (A), histidine (H), N6-[(2-azidoethoxy)carbonyl]-L-lysine, propargyl-L-lysine, azidohomoalanine, methionine (M), isoleucine (I), leucine (L); more preferably selected from alanine (A) or histidine (H).

6. The isolated recombinant fibrinogen of claim 1, wherein in the fibrinogen alpha-chains and/or the fibrinogen beta-chains and/or the fibrinogen gamma-chains at least two, preferably at least three, more preferably at least four, even more preferably at least five, at least six, at least seven or at least eight, or all, of said K and R residues are deleted or substituted, preferably substituted.

7. The isolated recombinant fibrinogen of claim 1, wherein
i. each fibrinogen alpha-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, identity with any of SEQ ID NOs: 6, 7, 8, 41, 42, 43, 45, 46, 47, 48, 49, and 50, preferably with SEQ ID NO: 6, or with SEQ ID NO: 7, or with SEQ ID NO: 8, or with SEQ ID NO: 41, or with SEQ ID NO: 42, or with SEQ ID NO: 43, more preferably with SEQ ID NO: 6, SEQ ID NO: 7, or SEQ ID NO: 8; and/or
ii. each fibrinogen beta-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, identity with SEQ ID NO: 9, or with SEQ ID NO: 44, preferably SEQ ID NO: 9; and/or
iii. each fibrinogen gamma-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, identity with SEQ ID NO: 10, or with SEQ ID NO: 11.

8. A fibrin sealant comprising isolated recombinant fibrinogen according to claim 1, and optionally thrombin.

9. The fibrin sealant according to claim 8 not comprising plasmin inhibitors.

10. A fibrin sealant kit comprising:
i) a container comprising isolated recombinant fibrinogen according to claim 1; and
ii) a container comprising, preferably human-derived, thrombin.

11. The fibrin sealant according to claim 8, further comprising at least one additive selected from the group consisting of protransglutaminase, preferably factor XIII (FXIII), calcium salts, preferably calcium chloride, polyphosphate (PolyP), Zn2+, fibronectin, hydroxyapatite, growth factors, preferably VEGF, one or more mono- and/or polysaccharides and cells, preferably stem cells.

12. Eucaryotic cell, comprising exogenous nucleotide sequences encoding one or more recombinant fibrinogen alpha-chains, one or more recombinant fibrinogen beta-chains, and one or more recombinant fibrinogen gamma-chains; wherein
i. preferably each of the one or more fibrinogen alpha-chains comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, identity with any of SEQ ID NOs: 6, 7, 8, 41, 42, 43, 45, 46, 47, 48, 49, and 50, preferably with SEQ ID NO: 6, or with SEQ ID NO: 7, or with SEQ ID NO: 8, or with SEQ ID NO: 41, or with SEQ ID NO: 42, or with SEQ ID NO: 43, more preferably with SEQ ID NO: 6, SEQ ID NO: 7, or SEQ ID NO: 8; and/or
ii. preferably each of the one or more fibrinogen beta-chains comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, identity with SEQ ID NO: 9, or with SEQ ID NO: 44, preferably with SEQ ID NO: 9; and/or
iii. preferably each of the one or more fibrinogen gamma-chain comprises, or consists of, an amino acid sequence, which, preferably excluding the one or more substituted or deleted K and R residues, has at least 80%, preferably at least 85%, more preferably at least 90%, even more preferably at least 92%, even more preferably at least 93%, even more preferably at least 94%, or 95%, or 96%, or 97%, or 98%, or 99%, or 99.5%, identity with SEQ ID NO: 10, or with SEQ ID NO: 11;
wherein
(a) in the fibrinogen alpha-chains, with reference to the respective positions in SEQ ID NO: 2, one or more lysine (K) and/or arginine (R) residues selected from the group consisting R38, R42, K97, K100, R114, R123, R129, R135, K210, R216, R218, K225, K227, K238, K243, K249, R258, R271, R287, R308, R353, R367, R425, R426, K432, K437, K440, R443, K446, K448, R458, R459, K463, K467, K480, R510, R512, K527, R547, K558, R573, K575, K581, R591, K599, K602, K620, R621, K625, R687 and R847; and/or
(b) in the fibrinogen beta-chains, with reference to the respective positions in SEQ ID NO: 3, one or more lysine (K) and/or arginine (R) residues selected from the group consisting of R44, K51, K52, R53, R60, R72, K77, K83, R87, K152, R158, K160, K163, K178, R285, K328, R334, K348, K353, K367, K374, K458 and K471 are substituted or deleted; and/or
(c) in the fibrinogen gamma-chains, with reference to the respective positions in SEQ ID NO: 4, one or more lysine (K) and/or arginine (R) residues selected from the group consisting of R40, K79, K84, K88, K111, K114, K185, K199, K238, K292, R301, K382 and K399 are substituted or deleted;
which, when cultured under conditions wherein the fibrinogen is produced, produces functional recombinant fibrinogen.

13. A method for the production of isolated recombinant fibrinogen, comprising the steps of
A) culturing eucaryotic cells comprising exogenous nucleotide sequences encoding at least one fibrinogen alpha-chain, at least one fibrinogen beta-chain, and at least one fibrinogen gamma-chain, preferably derived from mammalian, more preferably human, fibrinogen, in a culture medium under conditions wherein fibrinogen is produced; and
B) recovering the recombinant fibrinogen produced;
wherein step A) is carried out in the presence of at least one matrix metalloproteinase (MMP) inhibitor, preferably selected from the group consisting of 1,10 Phenanthroline, UK 370106, GM6001, and a combination thereof;
and wherein, optionally, the isolated recombinant fibrinogen is the isolated recombinant fibrinogen according to claim 1.

14. (canceled)

15. A method for identifying plasmin resistant recombinant fibrinogen variants comprising the steps of
A) Identification of plasmin cleavage sites in fibrinogen, by
i) incubating a fibrin gel, of preferably 10-30 mg/ml, more preferably 20 mg/ml density, with a plasmin solution of preferably 0.001 to 0.01 mU/ml;
ii) collecting supernatant at different timepoints after addition of the plasmin solution;
iii) determining cleavage sites in fibrinogen, preferably using mass spectrometry, to obtain a time-resolved cleavage pattern of fibrinogen; and
iv) identifying lysine (K) and/or arginine (R) residues immediately before, and optionally immediately after, the cleavage sites in the amino acid sequence of fibrinogen; and
B) optionally selecting one or more of the identified lysine (K) and/or arginine (R) residues in the amino acid sequence of fibrinogen, preferably based on the time-resolved cleavage pattern, for substitution or deletion, to obtain a new recombinant fibrinogen variant; and optionally incubating the new recombinant fibrinogen variant with plasmin to test for sensitive to proteolytic cleavage by plasmin;
optionally, wherein in step A) iii) an internal standard, preferably angiotensin II, is used for analysing the cleavage pattern.

16. The eucaryotic cell according to claim 12, wherein the eucaryotic cells are selected from mammalian cell culture cells, preferably Chinese hamster ovary (CHO) cells, PER.C6 cells, or human embryonic kidney (HEK) cells, or murine myeloma cells, or Baby hamster kidney (BHK) cells, or rat myeloma cells.

17. The the process for the production of recombinant fibrinogen according to claim 13, wherein the eucaryotic cells are selected from mammalian cell culture cells, preferably Chinese hamster ovary (CHO) cells, PER.C6 cells, or human embryonic kidney (HEK) cells, or murine myeloma cells, or Baby hamster kidney (BHK) cells, or rat myeloma cells.


SEQUENCE LISTING (82 sequences)

SEQ ID NO: 1 - protein, 644 aa, Homo sapiens
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRGIHTSPLGKPSLSP

SEQ ID NO: 2 - protein, 866 aa, Homo sapiens
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRDCDDVLQTHPSGTQSGIFNIKLPGSSKIFSVYCDQETSLGGWLLIQQRMDGSLNFNRTWQDYKRGFGSLNDEGEGEFWLGNDYLHLLTQRGSVLRVELEDWAGNEAYAEYHFRVGSEAEGYALQVSSYEGTAGDALIEGSVEEGAEYTSHNNMQFSTFDRDADQWEENCAEVYGGGWWYNNCQAANLNGIYYPGGSYDPRNNSPYEIENGVVWVSFRGADYSLRAVRMKIRPLVTQ

SEQ ID NO: 3 - protein, 491 aa, Homo sapiens
MKRMVSWSFHKLKTMKHLLLLLLCVFLVKSQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 4 - protein, 437 aa, Homo sapiens
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQAGDV

SEQ ID NO: 5 - protein, 453 aa, Homo sapiens
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

SEQ ID NO: 6 - protein, 625 aa, Homo sapiens
ADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRGIHTSPLGKPSLSP

SEQ ID NO: 7 - protein, 610 aa, Homo sapiens
ADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPV

SEQ ID NO: 8 - protein, 847 aa, Homo sapiens
ADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRDCDDVLQTHPSGTQSGIFNIKLPGSSKIFSVYCDQETSLGGWLLIQQRMDGSLNFNRTWQDYKRGFGSLNDEGEGEFWLGNDYLHLLTQRGSVLRVELEDWAGNEAYAEYHFRVGSEAEGYALQVSSYEGTAGDALIEGSVEEGAEYTSHNNMQFSTFDRDADQWEENCAEVYGGGWWYNNCQAANLNGIYYPGGSYDPRNNSPYEIENGVVWVSFRGADYSLRAVRMKIRPLVTQ

SEQ ID NO: 9 - protein, 461 aa, Homo sapiens
QGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 10 - protein, 411 aa, Homo sapiens
YVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQAGDV

SEQ ID NO: 11 - protein, 427 aa, Homo sapiens
YVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

SEQ ID NO: 12 - DNA, 44 bp, synthetic construct
  source 1..44: mol_type = other DNA
gcagctgatcagcgccatccagctgacctacaaccccgacgagg

SEQ ID NO: 13 - DNA, 43 bp, synthetic construct
  source 1..43: mol_type = other DNA
ggtcagctggatggcgctgatcagctgcttcacttcgctggtc

SEQ ID NO: 14 - DNA, 38 bp, synthetic construct
  source 1..38: mol_type = other DNA
ccgccaccctgagctcccggaagatgctggaagagatc

SEQ ID NO: 15 - DNA, 33 bp, synthetic construct
  source 1..33: mol_type = other DNA
cgggagctcagggtggcggcgtcgatcatgttg

SEQ ID NO: 16 - DNA, 37 bp, synthetic construct
  source 1..37: mol_type = other DNA
ctacagcagcgccagcacccccaacggctacgacaac

SEQ ID NO: 17 - DNA, 39 bp, synthetic construct
  source 1..39: mol_type = other DNA
gtgctggcgctgctgtaggtgccgccctggtagtacacg

SEQ ID NO: 18 - DNA, 39 bp, synthetic construct
  source 1..39: mol_type = other DNA
cacctggagcacccggtggtacagcatgaagaaaaccac

SEQ ID NO: 19 - DNA, 35 bp, synthetic construct
  source 1..35: mol_type = other DNA
ccgggtgctccaggtggcccagatgatgccgttgt

SEQ ID NO: 20 - DNA, 39 bp, synthetic construct
  source 1..39: mol_type = other DNA
tggcggccgctcgagtctaggccaccatggtgtccatgc

SEQ ID NO: 21 - DNA, 37 bp, synthetic construct
  source 1..37: mol_type = other DNA
ttgttcgaagggccctctagcctgagtgaccaggggc

SEQ ID NO: 22 - DNA, 39 bp, synthetic construct
  source 1..39: mol_type = other DNA
gccgccagtgtgctggaattgccaccatggtccggatgg

SEQ ID NO: 23 - DNA, 43 bp, synthetic construct
  source 1..43: mol_type = other DNA
tgatggatatctgcagaatttcactgctgaggaaagaatggcc

SEQ ID NO: 24 - DNA, 39 bp, synthetic construct
  source 1..39: mol_type = other DNA
gccgccagtgtgctggaattgccaccatggtctggtccc

SEQ ID NO: 25 - DNA, 39 bp, synthetic construct
  source 1..39: mol_type = other DNA
tgatggatatctgcagaatttcacaggtcgtcctcgggg

SEQ ID NO: 26 - DNA, 40 bp, synthetic construct
  source 1..40: mol_type = other DNA
gtattgggcgctcttccgctgtacgggccagatatacgcg

SEQ ID NO: 27 - DNA, 40 bp, synthetic construct
  source 1..40: mol_type = other DNA
cgcgtatatctggcccgtacccatagagcccaccgcatcc

SEQ ID NO: 28 - DNA, 40 bp, synthetic construct
  source 1..40: mol_type = other DNA
ggatgcggtgggctctatgggtacgggccagatatacgcg

SEQ ID NO: 29 - DNA, 40 bp, synthetic construct
  source 1..40: mol_type = other DNA
gagtcagtgagcgaggaagcccatagagcccaccgcatcc

SEQ ID NO: 30 - DNA, 43 bp, synthetic construct
  source 1..43: mol_type = other DNA
tgttcgaagggccctctagatcaagcctgagtgaccaggggcc

SEQ ID NO: 31 - DNA, 37 bp, synthetic construct
  source 1..37: mol_type = other DNA
ttgttcgaagggccctctagctagggggacagggaag

SEQ ID NO: 32 - DNA, 33 bp, synthetic construct
  source 1..33: mol_type = other DNA
taagaaggagatataccatggaagcagcagtcg

SEQ ID NO: 33 - DNA, 31 bp, synthetic construct
  source 1..31: mol_type = other DNA
catctggtcacttctaaaggagataaagagc

SEQ ID NO: 34 - DNA, 72 bp, synthetic construct
  source 1..72: mol_type = other DNA
catctggtcacttctcatggagatcatgagctcaggactggtcatgagcatgtcacctctggtagcacaacc

SEQ ID NO: 35 - DNA, 37 bp, synthetic construct
  source 1..37: mol_type = other DNA
cctttagaagtgaccagatgttctgtgtggtactctc

SEQ ID NO: 36 - DNA, 37 bp, synthetic construct
  source 1..37: mol_type = other DNA
ccatgagaagtgaccagatgttctgtgtggtactctc

SEQ ID NO: 37 - DNA, 23 bp, synthetic construct
  source 1..23: mol_type = other DNA
gagaacaagaccagcgaagtgaa

SEQ ID NO: 38 - DNA, 23 bp, synthetic construct
  source 1..23: mol_type = other DNA
cttcatggtggttttcttcatgc

SEQ ID NO: 39 - DNA, 23 bp, synthetic construct
  source 1..23: mol_type = other DNA
gtacagcatgaagaaaaccacca

SEQ ID NO: 40 - DNA, 23 bp, synthetic construct
  source 1..23: mol_type = other DNA
cttcacttcgctggtcttgttct

SEQ ID NO: 41 - protein, 609 aa, Homo sapiens
GPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRGIHTSPLGKPSLSP

SEQ ID NO: 42 - protein, 594 aa, Homo sapiens
GPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPV

SEQ ID NO: 43 - protein, 831 aa, Homo sapiens
GPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRDCDDVLQTHPSGTQSGIFNIKLPGSSKIFSVYCDQETSLGGWLLIQQRMDGSLNFNRTWQDYKRGFGSLNDEGEGEFWLGNDYLHLLTQRGSVLRVELEDWAGNEAYAEYHFRVGSEAEGYALQVSSYEGTAGDALIEGSVEEGAEYTSHNNMQFSTFDRDADQWEENCAEVYGGGWWYNNCQAANLNGIYYPGGSYDPRNNSPYEIENGVVWVSFRGADYSLRAVRMKIRPLVTQ

SEQ ID NO: 44 - protein, 447 aa, Homo sapiens
GHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 45 - protein, 269 aa, Homo sapiens
ADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRN

SEQ ID NO: 46 - protein, 297 aa, Homo sapiens
ADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTG

SEQ ID NO: 47 - protein, 309 aa, Homo sapiens
ADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGP

SEQ ID NO: 48 - protein, 253 aa, Homo sapiens
GPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRN

SEQ ID NO: 49 - protein, 281 aa, Homo sapiens
GPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTG

SEQ ID NO: 50 - protein, 293 aa, Homo sapiens
GPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGP

SEQ ID NO: 51 - protein, 7 aa, synthetic construct
QLIKAIQ

SEQ ID NO: 52 - protein, 7 aa, synthetic construct
QLIHAIQ

SEQ ID NO: 53 - protein, 7 aa, synthetic construct
QLIAAIQ

SEQ ID NO: 54 - protein, 7 aa, synthetic construct
ATLKSRM

SEQ ID NO: 55 - protein, 7 aa, synthetic construct
ATLHSRM

SEQ ID NO: 56 - protein, 7 aa, synthetic construct
ATLASRM

SEQ ID NO: 57 - protein, 7 aa, synthetic construct
TYSKAST

SEQ ID NO: 58 - protein, 7 aa, synthetic construct
TYSHAST

SEQ ID NO: 59 - protein, 7 aa, synthetic construct
TYSAAST

SEQ ID NO: 60 - protein, 7 aa, synthetic construct
ATWKTRW

SEQ ID NO: 61 - protein, 7 aa, synthetic construct
ATWHTRW

SEQ ID NO: 62 - protein, 7 aa, synthetic construct
ATWATRW

SEQ ID NO: 63 - protein, 44 aa, synthetic construct
SPGSGNARPNNPDWGTEEEVSGNVSPGTRREYHTEKLVTSKGDK

SEQ ID NO: 64 - protein, 8 aa, synthetic construct
QLIKAIQL

SEQ ID NO: 65 - protein, 7 aa, synthetic construct
ATLKSRK

SEQ ID NO: 66 - protein, 7 aa, synthetic construct
TYSKAST

SEQ ID NO: 67 - protein, 7 aa, synthetic construct
ATWKTRW

SEQ ID NO: 68 - protein, 8 aa, synthetic construct
QLIHAIQL

SEQ ID NO: 69 - protein, 7 aa, synthetic construct
ATLHSRK

SEQ ID NO: 70 - protein, 7 aa, synthetic construct
TYSHAST

SEQ ID NO: 71 - protein, 7 aa, synthetic construct
ATWHTRW

SEQ ID NO: 72 - protein, 8 aa, synthetic construct
QLIAAIQL

SEQ ID NO: 73 - protein, 7 aa, synthetic construct
ATLASRK

SEQ ID NO: 74 - protein, 7 aa, synthetic construct
TYSAAST

SEQ ID NO: 75 - protein, 7 aa, synthetic construct
ATWATRW

SEQ ID NO: 76 - protein, 236 aa, Homo sapiens
DCDDVLQTHPSGTQSGIFNIKLPGSSKIFSVYCDQETSLGGWLLIQQRMDGSLNFNRTWQDYKRGFGSLNDEGEGEFWLGNDYLHLLTQRGSVLRVELEDWAGNEAYAEYHFRVGSEAEGYALQVSSYEGTAGDALIEGSVEEGAEYTSHNNMQFSTFDRDADQWEENCAEVYGGGWWYNNCQAANLNGIYYPGGSYDPRNNSPYEIENGVVWVSFRGADYSLRAVRMKIRPLVTQ

SEQ ID NO: 77 - protein, 8 aa, synthetic construct
HTEKLVTS

SEQ ID NO: 78 - protein, 8 aa, synthetic construct
HTEKHVRS

SEQ ID NO: 79 - protein, 644 aa, synthetic construct
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKHVRSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRGIHTSPLGKPSLSP

SEQ ID NO: 80 - protein, 437 aa, synthetic construct
MSWSLHPRNLILYFYALLFLSSTCVAYVATRDNCCILDERFGSYCPTTCGIADFLSTYQTKVDKDLQSLEDILHQVENKTSEVKQLIHAIQLTYNPDESSKPNMIDAATLHSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSHASTPNGYDNGIIWATWHTRWYSMKKTTMKIIPFNRLTIGEGQQHHLGGAKQAGDV

SEQ ID NO: 81 - protein, 19 aa, synthetic construct
QLIHAIQLTYNPDESSKPN

SEQ ID NO: 82 - protein, 19 aa, synthetic construct
QLIAAIQLTYNPDESSKPN